SEQUENCE LISTING (95 sequences)

SEQ ID NO: 1 - DNA, 229 bp, artificial
  Inverted repeat
cagttgaagtcggaagtttacatacacttaagttggagtcattaaaactcgtttttcaactactccacaaatttcttgttaacaaacaatagttttggcaagtcagttaggacatctactttgtgcatgacacaagtcatttttccaacaattgtttacagacagattatttcacttataattcactgtatcacaattccagtgggtcagaagtttacatacactaagt

SEQ ID NO: 2 - DNA, 229 bp, artificial
  Inverted repeat
attgagtgtatgtaaacttctgacccactgggaatgtgatgaaagaaataaaagctgaaatgaatcattctctctactattattctgayatttcacattcttaaaataaagtggtgatcctaactgacctaagacagggaatttttactaggattaaatgtcaggaattgtgaaaasgtgagtttaaatgtatttggctaaggtgtatgtaaacttccgacttcaactg

SEQ ID NO: 3 - DNA, 32 bp, artificial
  Direct repeat
cagttgaagtcggaagtttacatacacytaag

SEQ ID NO: 4 - DNA, 32 bp, artificial
  Direct repeat
yccagtgggtcagaagtttacatacactwart

SEQ ID NO: 5 - protein, 340 aa, artificial
  SB polypeptide
MGKSKEISQDLRKKIVDLHKSGSSLGAISKRLKVPRSSVQTIVRKYKHHGTTQPSYRSGRRRVLSPRDERTLVRKVQINPRTTAKDLVKMLEETGTKVSISTVKRVLYRHNLKGRSARKKPLLQNRHKKARLRFATAHGDKDRTFWRNVLWSDETKIELFGHNDHRYVWRKKGEACKPKNTIPTVKHGGGSIMLWGCFAAGGTGALHKIDGIMRKENYVDILKQHLKTSVRKLKLGRKWVFQMDNDPKHTSKVVAKWLKDNKVKVLEWPSQSPDLNPIENLWAELKKRVRARRPTNLTQLHQLCQEEWAKIHPTYCGKLVEGYPKRLTQVKQFKGNATKY

SEQ ID NO: 6 - DNA, 229 bp, artificial
  Left inverted repeat
cagttgaagtcggaagtttacatacacttargttggagtcattaaaactcgtttttcaacyacwccacaaatttcttgttaacaaacwatagttttggcaagcragttaggacatctactttgtgcatgacacaagtmatttttccaacaattgtttacagacagattatttcacttataattcactgtatcacaattccagtgggtcagaagtttacatacactaagt

SEQ ID NO: 7 - DNA, 229 bp, artificial
  Right inverted repeat
ttgagtgtatgttaacttctgacccactgggaatgtgatgaaagaaataaaagctgaaatgaatcattctctctactattattctgayatttcacattcttaaaataaagtggtgatcctaactgaccttaagacagggaatctttactcggattaaatgtcaggaattgtgaaaaastgagtttaaatgtatttggctaaggtgtatgtaaacttccgacttcaactg

SEQ ID NO: 8 - DNA, 4732 bp, artificial
  Nucleotide sequence of pCMV/SB
gatccgacatcatgggaaaatcaaaagaaatcagccaagacctcagaaaaaaaattgtagacctccacaagtctggttcatccttgggagcaatttccaaacgcctgaaagtaccacgttcatctgtacaaacaatagtacgcaagtataaacaccatgggaccacgcagccgtcataccgctcaggaaggagacgcgttctgtctcctagagatgaacgtactttggtgcgaaaagtgcaaatcaatcccagaacaacagcaaaggaccttgtgaagatgctggaggaaacaggtacaaaagtatctatatccacagtaaaacgagtcctatatcgacataacctgaaaggccgctcagcaaggaagaagccactgctccaaaaccgacataagaaagccagactacggtttgcaactgcacatggggacaaagatcgtactttttggagaaatgtcctctggtctgatgaaacaaaaatagaactgtttggccataatgaccatcgttatgtttggaggaagaagggggaggcttgcaagccgaagaacaccatcccaaccgtgaagcacgggggtggcagcatcatgttgtgggggtgctttgctgcaggagggactggtgcacttcacaaaatagatggcatcatgaggaaggaaaattatgtggatatattgaagcaacatctcaagacatcagtcaggaagttaaagcttggtcgcaaatgggtcttccaaatggacaatgaccccaagcatacttccaaagttgtggcaaaatggcttaaggacaacaaagtcaaggtattggagtggccatcacaaagccctgacctcaatcctatagaaaatttgtgggcagaactgaaaaagcgtgtgcgagcaaggaggcctacaaacctgactcagttacaccagctctgtcaggaggaatgggccaaaattcacccaacttattgtgggaagcttgtggaaggctacccgaaacgtttgacccaagttaaacaatttaaaggcaatgctaccaaatactagaattggccgcggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagcttgcatgcctgcaggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggcc

SEQ ID NO: 9 - DNA, 15 bp, artificial
  SA site
  misc_feature (12)..(12): n is a, c, g, or t
cccccccccccncag

SEQ ID NO: 10 - DNA, 15 bp, artificial
  SA site
  misc_feature (12)..(12): n is a, c, g, or t
tttttttttttntag

SEQ ID NO: 11 - DNA, 34 bp, artificial
  loxP site
ataacttcgtatagcatacattatacgaagttat

SEQ ID NO: 12 - skipped

SEQ ID NO: 13 - DNA, 32 bp, artificial
  Left outer repeat
cagttgaagtcggaagtttacatacacttaag

SEQ ID NO: 14 - DNA, 32 bp, artificial
  Left inner repeat
tccagtgggtcagaagtttacatacactaagt

SEQ ID NO: 15 - DNA, 32 bp, artificial
  Left inner repeat
tccagtgggtcagaagtttacatacacttaag

SEQ ID NO: 16 - DNA, 32 bp, artificial
  Right inner repeat
cccagtgggtcagaagtttacatacactcaat

SEQ ID NO: 17 - DNA, 32 bp, artificial
  Right outer repeat
cagttgaagtcggaagtttacatacaccttag

SEQ ID NO: 18 - DNA, 5073 bp, artificial
  T2/Onc transposon
cagttgaagtcggaagtttacatacacttaagttggagtcattaaaactcgtttttcaactactccacaaatttcttgttaacaaacaatagttttggcaagtcagttaggacatctactttgtgcatgacacaagtcatttttccaacaattgtttacagacagattatttcacttataattcactgtatcacaattccagtgggtcagaagtttacatacactaagttgactgtgcctttaaacagcttggaaaattccagaaaatgatgtcatggctttagaagcttgattcgaggtcgacggtatcgagcttgatgatcccctagtttgtgataggccttttagctacatctgccaatccatctcattttcacacacacacacaccactttccttctggtcagtgggcacatgtccagcctcaagtttatatcaccacccccaatgcccaacacttgtatggccttgggcgggacatccccccccccacccccagtatctgcaacctcaagctagcttgggtgcgttggttgtggataagtagctagactccagcaaccagtaacctctgccctttctcctccatgacaaccaggtcccaggtcccgaaaaccaaagaagaagaacgcagatcgcagatctggactctagaggatcatcgaattctgcagtcgacggtaccgcgggcccgggatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgcgatggaatagggaaccgaatccccaccccacccccagcattctagttctgaagaccccagcgttgaggaccaagggtgcagtagccctggccctcagaggctctcagaggctccgcttgcagccagccttggcgctctttattacctgagagatcctcgcgggcgttctggtgcaagtagcaagcttgatgggcgacgcagtctatcggaggactggcgcgccgagtgaggggttgtgggctcttttattgagctcggggagcagaagcgcgcgaacagaagcgagaagcgaactgattggttagttcaaataaggcacagggtcattttcaggtccttggggcaccctggaaacatctgatggttctctagaaactgctgagggcgggaccgcatctggggaccatctgttcttggccctgagccggggcaggaactgcttaccacagatatcctgtttggcccatattctgctgtctctctgttcctaaccttgatctgaacttctctattctcagttatgtattttccatgccttgcaaaatggcgttacttaagctagcttgccaaacctacaggtggggtctttcattcccccctttttctggagactaaataaaatcttttattttatctatggctcgtactctataggcttcagatcgaattcctgcagcccgggggatccactagaattcccgcgaatccatctttcacataccggctacgttgctaacaaccagtgcggcaatttcatcatcggctgaactgtaaatgaatgagaaaaccggtttagaaagtgcacagctgtcagggaagtcaacacttcagtgagcatgtgaccatgtggagtcagcttcctgtttgtcctagttctagagcggccgctctagatggccagatctagcttgtggaaggctactcgaaatgtttgacccaagttaaacaatttaaaggcaatgctaccaaatactaattgagtgtatgtaaacttctgacccactgggaatgtgatgaaagaaataaaagctgaaatgaatcattctctctactattattctgatatttcacattcttaaaataaagtggtgatcctaactgacctaagacagggaatttttactaggattaaatgtcaggaattgtgaaaaagtgagtttaaatgtatttggctaaggtgtatgtaaacttccgacttcaactgtatagggatcctctagctagagtcgacctcgagggggggcccggtacccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggagctcggatccctata

SEQ ID NO: 19 - DNA, 4968 bp, artificial
  T2/Onc transposon with En2 SA
ccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggagctcggatccctatacagttgaagtcggaagtttacatacacttaagttggagtcattaaaactcgtttttcaactactccacaaatttcttgttaacaaacaatagttttggcaagtcagttaggacatctactttgtgcatgacacaagtcatttttccaacaattgtttacagacagattatttcacttataattcactgtatcacaattccagtgggtcagaagtttacatacactaagttgactgtgcctttaaacagcttggaaaattccagaaaatgatgtcatggctttagaagcttgatggccgctctagaactaggattgcagcacgaaacaggaagctgactccacatggtcacatgctcactgaagtgttgacttccctgacagctgtgcactttctaaaccggttttctcattcatttacagttcagccgatgatgaaattgccgcactggttgttagcaacgtagccggtatgtgaaagatggattcgcgggaatttagtggatcccccgggctgcaggaattcgatctgaagcctatagagtacgagccatagataaaataaaagattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccatcaagcttgctactagcaccagaacgcccgcgaggatctctcaggtaataaagagcgccaaggctggctgcaagcggagcctctgagagcctctgagggccagggctactgcacccttggtcctcaacgctggggtcttcagaactagaatgctgggggtggggtggggattcggttccctattccatcgcgcgttaagatacattgatgagtttggacaaaccacaactagaatgcagtgaaaaaaatgctttatttgtgaaatttgtgatgctattgctttatttgtaaccattataagctgcaataaacaagttggccgctcctgtgccagactctggcgccgctgctctgtcaggtacctgttggtctgaaactcagccttgagcctctggagctgctcagcagtgaaggctgtgcgaggccgcttgtcctctttgttagggttcttcttctttggttttcgggacctgggacctggttgtcatggaggagaaagggcagaggttactggttgctggagtctagctacttatccacaacccacgcacccaagcttgaggttgcagatactgggggtggggggggggggatgacccgcccaaggccatacaagtgttgggcattgggggtggtgatataaacttgaggctgggcatgtgcccactgaccagaaggaaagtggtgtgtgtgtgtgaaaatgagatggattggcagatgtagctaaaaggcctatcacaaactaggggatctagcttgtggaaggctactcgaaatgtttgacccaagttaaacaatttaaaggcaatgctaccaaatactaattgagtgtatgtaaacttctgacccactgggaatgtgatgaaagaaataaaagctgaaatgaatcattctctctactattattctgatatttcacattcttaaaataaagtggtgatcctaactgacctaagacagggaatttttactaggattaaatgtcaggaattgtgaaaaagtgagtttaaatgtatttggctaaggtgtatgtaaacttccgacttcaactgtatagggatcctctagctagagtcgacctcgagggggggcccggtacccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaattt

SEQ ID NO: 20 - protein, 340 aa, artificial
  SB polypeptide
  misc_feature (136)..(136): Arginine, a lysine, or a histidine
  misc_feature (243)..(243): Glutamine or an asparagine
  misc_feature (253)..(253): Arginine, a lysine, or a histidine
  misc_feature (255)..(255): Arginine, a lysine, or a histidine
MGKSKEISQDLRKKIVDLHKSGSSLGAISKRLKVPRSSVQTIVRKYKHHGTTQPSYRSGRRRVLSPRDERTLVRKVQINPRTTAKDLVKMLEETGTKVSISTVKRVLYRHNLKGRSARKKPLLQNRHKKARLRFAXAHGDKDRTFWRNVLWSDETKIELFGHNDHRYVWRKKGEACKPKNTIPTVKHGGGSIMLWGCFAAGGTGALHKIDGIMRKENYVDILKQHLKTSVRKLKLGRKWVFQXDNDPKHTSKXVXKWLKDNKVKVLEWPSQSPDLNPIENLWAELKKRVRARRPTNLTQLHQLCQEEWAKIHPTYCGKLVEGYPKRLTQVKQFKGNATKY

SEQ ID NO: 21 - protein, 340 aa, artificial
  SB polypeptide
MGKSKEISQDLRKKIVDLHKSGSSLGAISKRLKVPRSSVQTIVRKYKHHGTTQPSYRSGRRRVLSPRDERTLVRKVQINPRTTAKDLVKMLEETGTKVSISTVKRVLYRHNLKGRSARKKPLLQNRHKKARLRFARAHGDKDRTFWRNVLWSDETKIELFGHNDHRYVWRKKGEACKPKNTIPTVKHGGGSIMLWGCFAAGGTGALHKIDGIMRKENYVDILKQHLKTSVRKLKLGRKWVFQQDNDPKHTSKHVRKWLKDNKVKVLEWPSQSPDLNPIENLWAELKKRVRARRPTNLTQLHQLCQEEWAKIHPTYCGKLVEGYPKRLTQVKQFKGNATKY

SEQ ID NO: 22 - DNA, 32 bp, artificial
  Left outer repeat
cagttgaagtcggaagtttacatacacttrag

SEQ ID NO: 23 - DNA, 32 bp, artificial; identical to SEQ ID NO: 14
  Left inner repeat

SEQ ID NO: 24 - DNA, 31 bp, artificial
  Right inner direct repeat
cccagtgggtcagaagttaacatacactcaa

SEQ ID NO: 25 - DNA, 32 bp, artificial; identical to SEQ ID NO: 17
  Right outer repeat

SEQ ID NO: 26 - DNA, 1023 bp, artificial
  SB polynucleotide
atgggaaaatcaaaagaaatcagccaagacctcagaaaaaaaattgtagacctccacaagtctggttcatccttgggagcaatttccaaacgcctgaaagtaccacgttcatctgtacaaacaatagtacgcaagtataaacaccatgggaccacgcagccgtcataccgctcaggaaggagacgcgttctgtctcctagagatgaacgtactttggtgcgaaaagtgcaaatcaatcccagaacaacagcaaaggaccttgtgaagatgctggaggaaacaggtacaaaagtatctatatccacagtaaaacgagtcctatatcgacataacctgaaaggccgctcagcaaggaagaagccactgctccaaaaccgacataagaaagccagactacggtttgcaactgcacatggggacaaagatcgtactttttggagaaatgtcctctggtctgatgaaacaaaaatagaactgtttggccataatgaccatcgttatgtttggaggaagaagggggaggcttgcaagccgaagaacaccatcccaaccgtgaagcacgggggtggcagcatcatgttgtgggggtgctttgctgcaggagggactggtgcacttcacaaaatagatggcatcatgaggaaggaaaattatgtggatatattgaagcaacatctcaagacatcagtcaggaagttaaagcttggtcgcaaatgggtcttccaaatggacaatgaccccaagcatacttccaaagttgtggcaaaatggcttaaggacaacaaagtcaaggtattggagtggccatcacaaagccctgacctcaatcctatagaaaatttgtgggcagaactgaaaaagcgtgtgcgagcaaggaggcctacaaacctgactcagttacaccagctctgtcaggaggaatgggccaaaattcacccaacttattgtgggaagcttgtggaaggctacccgaaacgtttgacccaagttaaacaatttaaaggcaatgctaccaaatactag

SEQ ID NO: 27 - DNA, 1023 bp, artificial
  SB transposase
  misc_feature (406)..(406): A or C
  misc_feature (407)..(407): A or G
  misc_feature (408)..(408): Any nucleotide
  misc_feature (727)..(727): C or G
  misc_feature (728)..(728): A
  misc_feature (729)..(729): Any nucleotide
  misc_feature (757)..(757): A or C
  misc_feature (758)..(758): A or G
  misc_feature (759)..(759): Any nucleotide
  misc_feature (763)..(765): n is a, c, g, or t
  misc_feature (773)..(773): A or C
  misc_feature (774)..(774): A or G
  misc_feature (775)..(775): Any nucleotide
atgggaaaatcaaaagaaatcagccaagacctcagaaaaaaaattgtagacctccacaagtctggttcatccttgggagcaatttccaaacgcctgaaagtaccacgttcatctgtacaaacaatagtacgcaagtataaacaccatgggaccacgcagccgtcataccgctcaggaaggagacgcgttctgtctcctagagatgaacgtactttggtgcgaaaagtgcaaatcaatcccagaacaacagcaaaggaccttgtgaagatgctggaggaaacaggtacaaaagtatctatatccacagtaaaacgagtcctatatcgacataacctgaaaggccgctcagcaaggaagaagccactgctccaaaaccgacataagaaagccagactacggtttgcannngcacatggggacaaagatcgtactttttggagaaatgtcctctggtctgatgaaacaaaaatagaactgtttggccataatgaccatcgttatgtttggaggaagaagggggaggcttgcaagccgaagaacaccatcccaaccgtgaagcacgggggtggcagcatcatgttgtgggggtgctttgctgcaggagggactggtgcacttcacaaaatagatggcatcatgaggaaggaaaattatgtggatatattgaagcaacatctcaagacatcagtcaggaagttaaagcttggtcgcaaatgggtcttccaannngacaatgaccccaagcatacttccaaannngtgnnnaaatggcttaaggacaacaaagtcaaggtattggagtggccatcacaaagccctgacctcaatcctatagaaaatttgtgggcagaactgaaaaagcgtgtgcgagcaaggaggcctacaaacctgactcagttacaccagctctgtcaggaggaatgggccaaaattcacccaacttattgtgggaagcttgtggaaggctacccgaaacgtttgacccaagttaaacaatttaaaggcaatgctaccaaatactag

SEQ ID NO: 28 - DNA, 1023 bp, artificial
  SB transposase
atgggaaaatcaaaagaaatcagccaagacctcagaaaaaaaattgtagacctccacaagtctggttcatccttgggagcaatttccaaacgcctgaaagtaccacgttcatctgtacaaacaatagtacgcaagtataaacaccatgggaccacgcagccgtcataccgctcaggaaggagacgcgttctgtctcctagagatgaacgtactttggtgcgaaaagtgcaaatcaatcccagaacaacagcaaaggaccttgtgaagatgctggaggaaacaggtacaaaagtatctatatccacagtaaaacgagtcctatatcgacataacctgaaaggccgctcagcaaggaagaagccactgctccaaaaccgacataagaaagccagactacggtttgcaagagcacatggggacaaagatcgtactttttggagaaatgtcctctggtctgatgaaacaaaaatagaactgtttggccataatgaccatcgttatgtttggaggaagaagggggaggcttgcaagccgaagaacaccatcccaaccgtgaagcacgggggtggcagcatcatgttgtgggggtgctttgctgcaggagggactggtgcacttcacaaaatagatggcatcatgaggaaggaaaattatgtggatatattgaagcaacatctcaagacatcagtcaggaagttaaagcttggtcgcaaatgggtcttccaaatggacaatgaccccaagcatacttccaaacacgtgagaaaatggcttaaggacaacaaagtcaaggtattggagtggccatcacaaagccctgacctcaatcctatagaaaatttgtgggcagaactgaaaaagcgtgtgcgagcaaggaggcctacaaacctgactcagttacaccagctctgtcaggaggaatgggccaaaattcacccaacttattgtgggaagcttgtggaaggctacccgaaacgtttgacccaagttaaacaatttaaaggcaatgctaccaaatactag

SEQ ID NO: 29 - DNA, 19 bp, artificial
  Direct repeat
gtcrgaagtttacatacac

SEQ ID NO: 30 - DNA, 165 bp, artificial
  Intervening region
ttggagtcattaaaactcgtttttcaacyacwccacaaatttcttgttaacaaacwatagttttggcaagtcrgttaggacatctactttgtgcatgacacaagtmatttttccaacaattgtttacagacagattatttcacttataattcactgtatcacaat

SEQ ID NO: 31 - DNA, 166 bp, artificial
  Complement of intervening region
aatgtgatgaaagaaataaaagctgaaatgaatcattctctctactattattctgayatttcacattcttaaaataaagtggtgatcctaactgaccttaagacagggaatctttactcggattaaatgtcaggaattgtgaaaaastgagtttaaatgtatttgg

SEQ ID NO: 32 - DNA, 165 bp, artificial
  Intervening region
aatgtgatgaaagaaataaaagctgaaatgaatcattctctctactattattctgayatttcacattcttaaaataaagtggtgatcctaactgacctaagacagggaatttttactaggattaaatgtcaggaattgtgaaaasgtgagtttaaatgtatttgg

SEQ ID NO: 33 - DNA, 229 bp, artificial
  Complement of intervening region
cagttgaagtcggaagtttacatacacggggtttggagtcattaaaactcgtttttcaactactccacaaatttcttgttaacaaacaatagttttggcaagtcagttaggacatctactttgtgcatgacacaagtcatttttccaacaattgtttacagacagattatttcacttataattcactgtatcacaattccagtgggtcagaagtttacatacactaagt

SEQ ID NO: 34 - DNA, 18 bp, artificial
  Direct repeat
tcrgaagtttacatacac

SEQ ID NO: 35 - DNA, 18 bp, artificial
  T2/Onc excision primer
tgtgctgcaaggcgatta

SEQ ID NO: 36 - DNA, 18 bp, artificial
  T2/Onc excision primer
accatgattacgccaagc

SEQ ID NO: 37 - DNA, 40 bp, artificial
  Annealing primer
gtaatacgactcactatagggctccgcttaagggaccatg

SEQ ID NO: 38 - DNA, 18 bp, artificial
  Annealing primer
gtcccttaagcggtaaag

SEQ ID NO: 39 - DNA, 36 bp, artificial
  IR/DR annealing primer
gtaatacgactcactatagggctccgcttaagggac

SEQ ID NO: 40 - DNA, 17 bp, artificial
  IR/DR annealing primer
tagtcccttaagcggag

SEQ ID NO: 41 - DNA, 22 bp, artificial
  IR/DR flank annealing primer
gtaatacgactcactatagggc

SEQ ID NO: 42 - DNA, 32 bp, artificial
  IR/DR flank annealing primer
gcttgtggaaggctactcgaaatgtttgaccc

SEQ ID NO: 43 - DNA, 22 bp, artificial
  IR/DR flank annealing primer
gtaatacgactcactatagggc

SEQ ID NO: 44 - DNA, 36 bp, artificial
  IR/DR flank annealing primer
ctggaattttccaagctgtttaaaggcacagtcaac

SEQ ID NO: 45 - DNA, 18 bp, artificial
  IR/DR flank annealing primer
agggctccgctaagggac

SEQ ID NO: 46 - DNA, 31 bp, artificial
  IR/DR flank annealing primer
ccactgggaatgtgatgaaagaaataaaagc

SEQ ID NO: 47 - DNA, 18 bp, artificial
  IR/DR flank annealing primer
agggctccgctaagggac

SEQ ID NO: 48 - DNA, 29 bp, artificial
  IR/DR flank annealing primer
gacttgtgtcatgcacaaagtagatgtcc

SEQ ID NO: 49 - DNA, 20 bp, artificial
  Transposon primer
gtggtgatcctaactgacct

SEQ ID NO: 50 - DNA, 25 bp, artificial
  Braf insertion primers
cgtagttatcatttattggtagcag

SEQ ID NO: 51 - DNA, 20 bp, artificial
  Braf insertion primers
ggaaagctagatggaaattc

SEQ ID NO: 52 - DNA, 22 bp, artificial
  Braf insertion primers
ccatgcctgtgcatttgttatg

SEQ ID NO: 53 - DNA, 20 bp, artificial
  Braf insertion primers
gcacagatgcttaccatccg

SEQ ID NO: 54 - DNA, 22 bp, artificial
  Braf insertion primers
gcaaactctgtaataatgtacc

SEQ ID NO: 55 - DNA, 20 bp, artificial
  Braf insertion primers
ctaagcaggctgtttactac

SEQ ID NO: 56 - DNA, 20 bp, artificial
  Braf insertion primers
ctgtccccagtgaaatagtg

SEQ ID NO: 57 - DNA, 20 bp, artificial
  Braf insertion primers
ctcaagtgctgaagtttcag

SEQ ID NO: 58 - DNA, 24 bp, artificial
  Braf insertion primers
ataatccagtgataagaactgtgc

SEQ ID NO: 59 - DNA, 20 bp, artificial
  Braf insertion primers
cagccagtgcttataaactg

SEQ ID NO: 60 - DNA, 20 bp, artificial
  SD primer
gaacgcccgcgaggatctct

SEQ ID NO: 61 - DNA, 20 bp, artificial
  Braf exon primers
cttctgtcctccgaggatga

SEQ ID NO: 62 - DNA, 20 bp, artificial
  Braf exon primer
gagcatcacccagtaccaca

SEQ ID NO: 63 - DNA, 20 bp, artificial
  Carp SA primer
acgttgctaacaaccagtgc

SEQ ID NO: 64 - DNA, 20 bp, artificial
  Braf primer
cagtcctccgatagactgcg

SEQ ID NO: 65 - DNA, 20 bp, artificial
  Braf primer
ggactggctacttgaaggct

SEQ ID NO: 66 - DNA, 20 bp, artificial
  Geotyping primer
cagttgaagtcggaagttta

SEQ ID NO: 67 - DNA, 20 bp, artificial
  Geotyping primer
ggaattgtgatacagtgaat

SEQ ID NO: 68 - DNA, 16 bp, artificial
  Primer
ggatccactaaattcc

SEQ ID NO: 69 - DNA, 16 bp, artificial
  Primer
gttgactgtgccttta

SEQ ID NO: 70 - DNA, 40 bp, artificial; identical to SEQ ID NO: 37
  NlaIII linker

SEQ ID NO: 71 - DNA, 15 bp, artificial
  NlaIII linker
gtcccttaagcggag

SEQ ID NO: 72 - DNA, 36 bp, artificial; identical to SEQ ID NO: 39
  BfaI linker

SEQ ID NO: 73 - DNA, 17 bp, artificial
  BfaI linker
tagtcccttaagcggag

SEQ ID NO: 74 - DNA, 22 bp, artificial
  Linker primer
gtaatacgactcactatagggc

SEQ ID NO: 75 - DNA, 32 bp, artificial; identical to SEQ ID NO: 42
  IRDRR1 primer

SEQ ID NO: 76 - DNA, 36 bp, artificial; identical to SEQ ID NO: 44
  IRDRL1 primer

SEQ ID NO: 77 - DNA, 19 bp, artificial
  Linker rested primer
agggctccgcttaagggac

SEQ ID NO: 78 - DNA, 31 bp, artificial; identical to SEQ ID NO: 46
  IRDRR2 primer

SEQ ID NO: 79 - DNA, 29 bp, artificial
  IRDRL2 primer
gacttgtgtcatgcacaaagtagatgtcc

SEQ ID NO: 80 - DNA, 20 bp, artificial
  SDF primer
ctactagcaccagaacgccc

SEQ ID NO: 81 - DNA, 16 bp, artificial
  26f primer
tggaccccatggacat

SEQ ID NO: 82 - DNA, 20 bp, artificial
  29r primer
tgcagtcagcatccacctcc

SEQ ID NO: 83 - DNA, 20 bp, artificial
  SAr primer
catctttcacataccggcta

SEQ ID NO: 84 - DNA, 20 bp, artificial
  B-actin forward primer
gtgggccgccctaggcacca

SEQ ID NO: 85 - DNA, 20 bp, artificial
  B-actin rev primer
ctctttgatgtcacgcacga

SEQ ID NO: 86 - DNA, 20 bp, artificial
  rasgrp1 primer
gctgatattttcactgggga

SEQ ID NO: 87 - DNA, 20 bp, artificial
  rasgrp1 primer
cctgcgtgaatagaccctga

SEQ ID NO: 88 - DNA, 20 bp, artificial
  Runx2 primer
aactgcctggggtctgaaaa

SEQ ID NO: 89 - DNA, 20 bp, artificial
  Runx2 primer
cctcagtgatttagggcgca

SEQ ID NO: 90 - DNA, 20 bp, artificial
  Sox8 primer
gctccgtcttgatctgtggc

SEQ ID NO: 91 - DNA, 20 bp, artificial
  Sox8 primer
gaccaccacacaggccagac

SEQ ID NO: 92 - DNA, 34 bp, artificial
  Splice donor region
ccgcgaggatctctcaggtgagccggtggagcct

SEQ ID NO: 93 - DNA, 34 bp, artificial
  Splice acceptor region
gattgaggccgtgaagattcagccgatgatgaaa

SEQ ID NO: 94 - DNA, 28 bp, artificial
  SB11 primer
atgggaaaatcaaaagaaatcagccaag

SEQ ID NO: 95 - DNA, 33 bp, artificial
  SB11 primer
gccaaacagttctatttttgtttcatcagacca

(19) United States
(12) Patent Application Publication
(10) Pub. No.: US 2006/0026699 A1
(43) Pub. Date: Feb. 2, 2006

(54) Methods and compositions for identification of genomic sequences

(76) Inventors: David A. Largaespada, Mounds View, MN (US); Adam J. Dupuy, Walkersville, MD (US); Lara S. Collier, Roseville, MN (US); Neal G. Copeland, Ijamsville, MD (US); Nancy A. Jenkins, Ijamsville, MD (US)

Correspondence Address: MUETING, RAASCH & GEBHARDT, P.A., P.O. BOX 581415, MINNEAPOLIS, MN, 55458, US

(21) Appl. No.: 11/145,532

(22) Filed: Jun. 3, 2005

Related U.S. Application Data
(60) Provisional application No. 60/577,000, filed on Jun. 4, 2004.

(51) Int. Cl. A01K 67/027; C12Q 1/68

(52) U.S. Cl. 800/10; 435/6

(57) ABSTRACT

Methods of using a transposon as an insertional mutagen are provided. Also provided is a transgenic animal that includes polynucleotides encoding a transposon and transposase that can be used to identify genomic sequences. The methods and transgenic animals may be used to detect cancer-related genes by identifying common insertion sites in tumor cells.

This application claims the benefit of U.S. Provisional Application Ser. No. 60/577,000, filed Jun. 4, 2004, which is incorporated by reference herein.

### GOVERNMENT FUNDING

The present invention was made with government support under Grant No. RO1 DA014764, awarded by the NIH-NIDA. The Government may have certain rights in this invention.

### BACKGROUND

DNA transposons are mobile elements that can move from one position in a genome to another. Naturally, transposons play roles in evolution as a result of their movements within and between genomes. Geneticists have used transposons as tools for both gene delivery and insertional mutagenesis or gene tagging in lower animals (Shapiro, Genomics, 1992; 86:99-111) but not, until recently, in vertebrates. Transposons are relatively simple genetic systems, consisting of some genetic sequence bounded by inverted terminal repeats and a transposase enzyme that acts to cut the transposon out of one source of DNA and paste it into another DNA sequence (Plasterk, Cell, 1993; 74:781-786). Transposons operating by a copy and paste mechanism are also known. Autonomous transposons carry the transposase gene inside the transposon whereas non-autonomous transposons require another source of transposase for their mobilization.

Among the DNA transposable elements, members of Tc1/mariner family have been found in a wide variety of organisms, ranging from fungi to humans (Doak et al., Proc. Natl. Acad. Sci. USA, 1994; 91:942-946; Radice et al., Mol. Gen. Genet., 1994; 244:606-612). Both the Tc1 and mariner transposons can be transposed using purified transposase protein (Lampe et al., EMBO J., 1996; 15:5470-5479; Vos et al., Genes Dev., 1996; 10:755-761; Tosi et al., Nucl. Acids Res., 2000; 28:784-790). Tc1/mariner transposons are simple structures consisting of inverted terminal repeats (ITRs) that flank a single transposase gene. Transposase binds at precise sites in each of the ITRs where it cuts out the transposon and inserts it into a new DNA locus (a “cut-and-paste” mechanism). This simplicity in mechanism and broad range of invasion suggested that such a transposon would be useful to develop into a vertebrate transformation vector. However, all of the Tc1/mariner-type transposon genes available in vertebrate genomes have been extensively mutated, leaving them as repetitive, inactive DNA sequences (Izsvak et al., Mol. Gen. Genet., 1995; 247:312-322). An intensive search for transposons in vertebrates—primarily fish—did not result in the discover of a single active Tc1/mariner-type transposon (Izsvák et al., Mol. Gen. Genet., 1995; 247:312-322; Ivics et al., Proc. Natl. Acad. Sci. USA, 1996; 93:5008-5013). Of the nearly 10,000 Tc1/mariner-type transposons found in the haploid human genome, none appear to have active transposase genes (Lander et al., Nature, 2001; 409:860-921; Venter et al., Science, 2001; 291:1304-1351).

As a functional Tc1/mariner-type transposon could not be found in nature, a functional Tc1-like transposon system was instead reconstructed from sequences found in salmonid fish. This synthetic transposase was named Sleeping Beauty (SB), owing to its restoration from an inactive transposon that had essentially been “asleep” for more than 10 million years (Ivics et al., Cell, 1997; 91:501-510). The SB transposon appears to obey a cardinal rule of Tc1/mariner transposons; namely, it integrates only into a TA-dinucleotide sequence, which is duplicated upon insertion in the host genome (Ivics et al., Cell, 1997; 91:501-510; Luo et al., Proc. Natl. Acad. Sci. USA, 1998; 95:10769-10773). While the transposase is named Sleeping Beauty, the SB system actually consists of two parts: the SB transposase and a transposon that is responsive to SB transposase. Transposons in the Tc1/mariner superfamily can be sorted into three groups based on the different length of ITRs and the different numbers and patterns of transposase-binding sites in the ITRs (Plasterk et al., Trends Genet., 1999; 15:326-332). One group of transposons, which includes transposons of the SB system, have a structure that includes two ITR (inverted terminal repeats), each of which includes an IR/DR structure consisting of direct repeat (DR) sequences and inverted repeat (IR) sequences. (Ivics et al., Proc. Natl. Acad. Sci. USA, 1996; 93:5008-5013; Ivics et al., Cell, 1997; 91:501-510) The IR/DR structure includes a pair of binding-sites containing short, 15-20 bp DRs at the ends of each IR, which are about 200-250 bp in length. Both binding sites are essential for transposition—deletion or mutation of either DR or ITR virtually abolishes transposition (Ivics et al., Cell, 1997; 91:501-510; Izsvák et al., J. Mol. Biol., 2000; 302:93-102).

The SB system is functional in a wide range of vertebrate cells, from fish to humans (Plasterk et al., Trends Genet., 1999; 15:326-332; Izsvak et al., J. Mol. Biol., 2000; 302:93-102). It has been used to deliver genes for long-term gene expression in mice (Yant et al., Nature Genet., 2000; 25:35-40; Fischer et al., Proc. Natl. Acad. Sci. USA, 2001; 98:6759-6764; Dupuy et al., Genesis, 2001; 30:82-88; Dupuy et al., Proc. Natl. Acad. Sci. USA, 2002; 99:4495-4499; Horie et al., Proc. Natl. Acad. Sci. USA, 2001; 98:9191-9196) and in zebrafish. The SB system is nearly 10-fold more efficient than systems using other Tc1/mariner-type transposons in human cells (Fischer et al., Proc. Natl. Acad. Sci. USA, 2001 98:6759-6764), although the efficiency drops off as the size of the transposon increases (Izsvak et al., J. Mol. Biol., 2000; 302:93-102; Karsi et al., Mar. Biotechnol., 2001; 3:241-245). These findings suggest that the SB system has potential as a tool for transgenesis and insertional mutagenesis in vertebrates, as well as gene therapy in humans.

Insertional mutagenesis also has the potential to detect genes related to cancer. Most, if not all, cancer cells contain genetic damage that appears to be the responsible event leading to tumorigenesis. The genetic damage present in a parental tumorigenic cell is maintained as a heritable trait in subsequent generations of the tumorigenic cell line. The genetic damage found in cancer cells generally is found in two types of genes: proto-oncogenes, and tumor suppressor genes. However, damage to other genes, such as those governing immunity, cell motility, or angiogenesis, can also relate to cancer development.

A proto-oncogene is a gene whose protein product has the capacity to induce cellular transformation given it sustains some genetic insult. The distinction between the terms proto-oncogene and oncogene relates to the activity of the protein product of the gene. An oncogene is a gene that has sustained some genetic damage and, therefore, produces a protein capable of cellular transformation. The process of activation of proto-oncogenes to oncogenes can include retroviral transduction or retroviral integration (see below), point mutations, insertion mutations, gene amplification, chromosomal translocation and/or protein-protein interactions. Proto-oncogenes can be classified into many different groups based upon their normal function within cells or based upon sequence homology to other known proteins. Tumor suppressor genes, on the other hand, are genes that generally function to prevent cellular transformation, but can lose this capacity through genetic damage. Tumor suppressor genes also include growth suppressor genes, recessive oncogenes, and anti-oncogenes.

Given the complexity of inducing and regulating cellular growth, proliferation and differentiation, it was suspected for many years that genetic damage to genes encoding growth factors, growth factor receptors and/or the proteins of the various signal transduction cascades would lead to cellular transformation. This suspicion was confirmed with the identification of numerous genes, many of whose products function in cellular signaling, that are involved in some way in the genesis of the tumorigenic state. The majority of these proto-oncogenes have been identified by retroviral transformation or through transfection of DNA from tumor cell lines into non-transformed cell lines and screening for resultant tumorigenesis.

Radiation and chemical mutagens can induce cancer in mice by causing somatic cell mutations in cancer genes. For example, ethylnitrosourea (ENU) is being used to screen for dominant and recessive mutations (Nolan et al., Nat. Genet. 2000; 25:440-443). However, these methods result in tumors in which the identity of the mutated cancer genes cannot be readily identified. In other words, these methods do not provide for any “landmark” that can be used to find the involved genes. In the absence of such landmarks, scientists have tended to study one cancer gene at a time using gene knockouts, for tumor suppressor genes, or transgenes to overexpress oncogenes. Candidate tumor suppressor genes and oncogenes have been identified by a variety of methods over the last 25 years. One method to find candidate leukemia and mammary carcinoma genes has been the identification of proto-oncogenes at common sites of retroviral insertion in tumors from mice chronically infected with Murine Leukemia Viruses (MuLV) or Mouse Mammary Tumor Viruses (MMTV). Unfortunately, these viruses cannot be used to induce other types of cancer.

Retroviruses, by acting as somatic cell insertional mutagens, have been used to accelerate tumor formation in cancer predisposed mouse models (Lund et al., Nat. Genet. 2002; 32:160-5; Blaydes et al., J. Virol., 2001; 75:9427-34). Recurrent or common retroviral integration sites in tumor genomic DNA have indicated the chromosomal location of tumor suppressors and oncogenes (Jonkers et al., Biochem. Biophys. Acta., 1996; 1287:29-57). Proviruses that land within coding regions can result in loss-of-function mutations and thus have been used to identify tumor suppressors (Largaespada et al., J. Virol.; 69:5095-102). Retroviruses have been used to identify cancer genes in the hematopoietic system and mammary gland, but their use in other cell types has been limited (Neil et al., Cancer Cell 2002; 2:253-5; Johansson et al., Proc. Natl. Acad. Sci. USA, 2004; 101:11334-7). However, these methods suffer from an inability to easily modify the retroviral structure so that reporter constructs could be used, difficulty in generating a large number of new insertions, and/or a high degree of technical difficulty.

Another strategy is to generate large libraries of embryonic stem cell clones, each harboring a plasmid or retroviral gene trap insertion (Zambrowicz et al., Nature, 1998; 392:608-611). These libraries can be used for sequence-driven functional annotation of the mouse genome. The biological function of genes of interest, based on their sequences, can be studied by thawing the correct embryonic stem cell clone, injecting these cells into blastocysts, and passing the mutation through the germline to generate heterozygous and then homozygous gene mutations. However, the phenotype caused by disruption of a given gene cannot often be guessed from its sequence alone.

Transposon-tagged mutagenesis has proven to be useful for functional genomic screens in organisms such as Drosophila melanogaster (Spradling et al., Proc. Natl. Acad. Sci. USA, 1995; 92:10824-10830), Caenorhabditis elegans (Plasterk, Curr. Top. Microbiol. Immunol. 1996; 204:125-143) and plants (Osborne et al., Curr. Opin. Cell Biol., 1995; 7:406-413) but the lack of active elements in higher eukaryotes has precluded their use for mammalian functional genomics. Progress towards the development of transposons useful in mammalian studies was made when SB, particularly more active mutant forms of SB, were developed. The development of improved transposons and transposases is described by Hackett et al. in U.S. Patent App. No. 2004/0077572. SB is active in the mouse germline (Dupuy et al, Genesis, 2001; 30:82-88), at a rate of 1-2 transpositions per animal born, and mouse somatic cells (Carlson et al., Genetics, 2003; 165:243-256) but the transposition frequency is too low to be useful for most genetic screens.

Analysis of SB transposition integration sites cloned from the mouse germline indicates that SB has fewer transposition site biases than retrotransposons, increasing its potential as an insertional mutagen (Horie et al., Mol. Cell Biol., 2003; 23:9189-9207). SB does, however, show a small but significant bias toward genes and their upstream regulatory sequences, although this bias is much less than that observed with retroviruses (Yant et al., Mol. Cell Biol., 2005; 25:2085-2094). SB elements are also not locked in place following transposition and can continuously transpose to new sites. A limitation of SB is that transposed elements tend to reintegrate at sites linked to the donor site. Previous studies showed that 50-80% of germline SB transpositions are located within 10-25-megabase of the donor site (Horie et al., Proc. Natl. Acad. Sci. USA, 2001; 98:9191-9196; Carlson et al., Genetics, 2003; 165:243-256).

### SUMMARY OF THE INVENTION

The present invention represents a significant advance in the ability to make tumors in an animal and characterize the molecular events causing tumorigenesis. The experiments described provide the first non-viral insertional mutagen that efficiently induces tumors in mice. Transposition can easily be controlled to mutagenize a specific target tissue by simply restricting the site of transposase expression. Transposition can be adapted to generate virtually any kind of cancer by restricting the sites and/or timing of transposase expression. The high frequency of transposition possible with the methods described herein is expected to make it possible to model various types of human cancer without any knowledge of the causative events, and in a more unbiased manner than can be done with currently available methods. Cancer genes and their pathways associated with tumorigenesis can be rapidly identified, providing insight into human cancer through the use of animal models. Given the unexpectedly high somatic transposition frequencies achieved, there is no theoretical reason why transposition frequencies cannot be increased in the mouse germ line to levels that would permit efficient forward genetic screens using the methods of the present invention. Since the transposon tags the mutated gene, the gene is much easier to clone than a gene mutated by a point mutagen like ENU. Finally, uses of transposons such as SB are not restricted to the mouse. SB was originally isolated from fish and has already been shown to function in Zebrafish (Davidson et al., Dev. Biol., 2003; 263:191-202) and Medaka (Grabher et al., Gene, 2003; 322:57-66). Therefore, SB will be useful in forward genetic screens in any higher eukaryote where transgenesis is possible.

Accordingly, the present invention provides a method for characterizing an insertional mutation in a tumor-bearing mammal. The method includes providing a transgenic mammal, obtaining a tumor cell from a tumor on the transgenic mammal, and identifying the location of a mobilized transposon in the genomic DNA of the tumor cell. A cell of the transgenic mammal used in this method includes a polynucleotide that includes a coding region encoding a transposase, and a transposon that includes a polynucleotide, or complement thereof, including an insertional mutagen flanked by first and second inverted repeats, wherein the inverted repeats can bind to a transposase and the transposon is capable of integrating into genomic DNA in a cell. In once aspect of this method, the first inverted repeat includes a first outer direct repeat and a first inner direct repeat, the first outer direct repeat having a nucleotide sequence having at least about 80% identity to SEQ ID NO:3, and the first inner direct repeat having a nucleotide sequence having at least about 80% identity to SEQ ID NO:4, and each direct repeat binds an SB polypeptide. Furthermore, in this aspect of the method, the second inverted repeat includes a second inner direct repeat and a second outer direct repeat, the second inner direct repeat being the complement of a nucleotide sequence having at least about 80% identity to SEQ ID NO:4, and the second outer direct repeat being the complement of a nucleotide sequence having at least about 80% identity to SEQ ID NO:3, and each direct repeat binds an SB polypeptide. The transposase in this aspect of the invention is an SB transposase. In an additional aspect of the method, the transposase includes an amino acid sequence having at least about 80% identity with SEQ ID NO:21.

The method for characterizing an insertional mutation in a tumor-bearing mammal may further include the step of identifying the location of a mobilized transposon by determining the nucleotide sequences adjacent to the mobilized transposon. In a further aspect, the locations of a plurality of mobilized transposon are identified in the genomic DNA of the tumor cell. The tumor cells may be obtained from a single mammal, or the tumor cells may be obtained from a plurality of transgenic mammals. If the tumor cells are obtained from different mammals, the method may include the further step of comparing the locations of mobilized transposon from tumors obtained from different transgenic mammals to identify the location of a common insertion site. Transgenic mammals used in the method may be genetically predisposed to develop cancer. Many mutations that predispose an animal to cancer are known and readily available, and the present invention is not limited by the type of mutation that can be used to predispose an animal to cancer. Such mutations include, for instance, those resulting in increased expression and/or activity of an oncogene, and those resulting in decreased expression and/or activity of a tumor suppressor. The insertional mutagen used in the method for characterizing an insertional mutation may include an affective sequence, a disruptive sequence, or a combination thereof in an aspect of the invention. Furthermore, the insertional mutagen may include a splice acceptor site, a promoter, a splice donor site, a transcription terminator, or a combination thereof. In a preferred aspect of the method, the tumor is a solid tumor.

In a further aspect, the invention provides a method for identifying a common insertion site that includes identifying the location of a mobilized transposon in the genomic DNA of a tumor cell from a first transgenic mammal and a second transgenic mammal and comparing the location of the mobilized transposon obtained from the genomic DNA of the first and second transgenic mammals. The presence of the mobilized transposon in the same genomic region in both transgenic mammals, as identified by this method, indicates the genomic region is a common insertion site. Identifying the location of a mobilized transposon includes providing a first and second transgenic mammal, wherein a cell of each of the transgenic mammals includes a polynucleotide comprising a coding region encoding a transposase, and a transposon that includes a polynucleotide, or complement thereof, having an insertional mutagen flanked by first and second inverted repeats, in which the inverted repeats can bind to the transposase and the transposon is capable of integrating into genomic DNA in a cell. Furthermore, identification of the location of a mobilized transposon includes obtaining genomic DNA from a tumor cell from the first and second transgenic mammal and determining the nucleotide sequences adjacent to the mobilized transposon to identify the location of the mobilized transposon in the genomic DNA of the tumor cell from the first and second transgenic mammals.

The method for identifying a common insertion site may further include a first inverted repeat that includes a first outer direct repeat and a first inner direct repeat, the first outer direct repeat having a nucleotide sequence having at least about 80% identity to SEQ ID NO:3, and the first inner direct repeat having a nucleotide sequence having at least about 80% identity to SEQ ID NO:4, in which each direct repeat binds an SB polypeptide. Furthermore, the method includes a second inverted repeat that includes a second inner direct repeat and a second outer direct repeat, the second inner direct repeat being the complement of a nucleotide sequence having at least about 80% identity to SEQ ID NO:4, and the second outer direct repeat being the complement of a nucleotide sequence having at least about 80% identity to SEQ ID NO:3, in which each direct repeat binds an SB polypeptide. The transposase in this aspect of the invention is an SB transposase.

In further aspect of the method for identifying a common insertion site, the transposase may include an amino acid sequence having at least about 80% identity with SEQ ID NO:21. Additionally, the insertional mutagen may include an affective sequence and a disruptive sequence. The affective sequence may optionally include a splice donor and a promoter, and the disruptive sequence may optionally include a splice acceptor operably linked to a transcription termination signal site. In a further aspect of the invention, the insertional mutagen includes nucleotides 533 to 630, 807 to 1207, 1217 to 1394, 1444-1525, and 1686 to 1959 of SEQ ID NO:19 (i.e., the pT2/Onc2 transposon vector).

In yet another aspect, the common insertion site identified by the method includes the integration of two mobilized transposons identified from tumor cells obtained from two transgenic mammals that are within about 13 kilobases of each other. Alternately, the method includes use of a third transgenic mammal, and the common insertion site includes the integration of three mobilized transposons identified from tumor cells obtained from three transgenic mammals that are within about 269 kilobases of each other. Finally, in another aspect of the method, the common insertion site has a high probability of being a nucleotide sequence within a tumor-associated gene.

Unless otherwise specified, “a,” “an,” “the,” and “at least one” are used interchangeably and mean one or more than one.

Furthermore, the terms “comprises” and variations thereof do not have a limiting meaning where these terms appear in the description and claims.

### BRIEF DESCRIPTION OF THE FIGURES

FIG. 1. Schematic representation of a transposon. A transposon is depicted with nucleic acid sequence flanked by one inverted repeat on each side. The inverted repeat on the left or 5′ side of the transposon includes SEQ ID NO:6 (the nucleotide sequence in bold), with the left outer repeat (SEQ ID NO:22) and left inner repeat (SEQ ID NO:23) underlined. The inverted repeat on the right or 3′ side of the transposon includes SEQ ID NO:7 (the nucleotide sequence in italics), with the right outer repeat and right inner repeats present in the complementary strand underlined. Thus, the nucleotide sequence of the right inner direct repeat is 5′-CCCAGTGGGTCAGAAGTTAACATACACTCAA (SEQ ID NO:24), and the nucleotide sequence of the right outer repeat is 5′-CAGTTGAAGTCGGAAGTTTACATACACCTTAG (SEQ ID NO:25)

FIG. 2. (A) An annotated map of the pT2/Onc plasmid; (B) A listing of the pT2/Onc plasmid nucleotide sequence (SEQ ID NO:18).

FIG. 3. A listing of the pT2/Onc2 plasmid nucleotide sequence (SEQ ID NO:19).

FIG. 4. A pictorial version of the T2/Onc transposon (SEQ ID NO: 18) including the insertional mutagen elements within the flanked region of the transposon in one embodiment of the invention.

FIG. 5. A pictorial representation of an oncogene-containing transposon that can be used to deliver activated oncogenes to soma to stimulate tumor formation.

FIG. 6. (A) is a double-stranded nucleic acid sequence encoding an SB polypeptide (SEQ ID NO:26). (B) is the amino acid sequence (SEQ ID NO:5) of an SB transposase. The major functional domains are highlighted; NLS, a bipartite nuclear localization signal; the boxes marked D and E including the DDE domain (Doak, et al., Proc. Natl. Acad, Sci. USA, 1994; 91:942-946) that catalyzes transposition; DD(34)E box, a catalytic domain containing two invariable aspartic acid residues, D(153) and D(244), and a glutamic acid residue, E(279), the latter two separated by 43 amino acids.

FIG. 7. (A) is a nucleotide sequence (SEQ ID NO:27) encoding an SB transposase (SEQ ID NO:20). (B) is the amino acid sequence for SEQ ID NO:20, which is identical to SEQ ID NO:5, but SEQ ID NO:20 has an arginine, a lysine, or a histidine at position 136, a glutamine or a asparagine at position 243, an arginine, a lysine, or a histidine at position 253, and an arginine, a lysine, or a histidine at position 255. (C) is a nucleotide sequence (SEQ ID NO:28) encoding an SB transposase (SEQ ID NO:21). (D) is the amino acid sequence for SEQ ID NO:21, which is identical to SEQ ID NO:5, but SEQ ID NO:20 has an arginine at position 136, a glutamine at position 243, a histidine at position 253, and an arginine at position 255.

FIG. 8 is a pictorial representation showing the use of transgenic animals in which one animal containing a transposon in a germ cell is crossed with another animal containing a polynucleotide sequence encoding a transposase to provide a doubly transgenic animal FIG. 9 schematically shows the gain of function insertions into Braf in P19 Arf−/− sarcomas that resulted from mobilization of the transposon of the invention in a number of tumor-bearing animals.

FIG. 10. Vector design and somatic transposition. (A) The T2/Onc transposon contains elements to elicit transcriptional activation (MSCV 5′ LTR and splice donor [SD]) and inactivation (splice acceptors [SA] and polyadenylation signals [pA]). (B) A PCR excision assay demonstrates somatic transposon excision within mice doubly transgenic for transposon and transposase.

FIG. 11. Arf−/−; T2/Onc; CAGGS-SB10 mice have a reduced tumor latency compared to singly transgenic controls. (A) Kaplan-Meier survival curve comparing time to morbidity for Arf−/−; T2/Onc; CAGGS-SB10 mice (▴), Arf−/−; CAGGS-SB10 mice (●), and Arf−/−; T2/Onc mice (▪). (B and C) Examples of sarcomas from Arf−/−; T2/Onc; CAGGS-SB10 mice (B) Spindle cell tumor (undifferentiated sarcoma) found growing on the hindlimb. (C) Soft tissue sarcoma infiltrating stomach glands (arrow).

FIG. 12. Activation of Braf by T2/Onc insertion. (A) Position and orientation of Braf T2/Onc insertions (grey). Braf exons are indicated by vertical black lines. The ninth intron is expanded to show detail of insertions. (B) Three-primer PCR for ninth intron Braf T2/Onc insertions demonstrates tumor-specificity of insertions. (C) RT-PCR reveals the presence of fusion transcripts, present in several T2/Onc; CAGGS-SB10 tumors that harbor ninth intron insertions. (D) Western analysis detects a truncated C-terminal kinase domain of the BRAF protein (arrow) in sarcomas that harbor ninth intron Braf integrations. Full-length BRAF protein is also detected (arrowhead). Total ERK was used as a loading control. (E) The SD-Braf transcript was amplified from tumors, cloned into an expression vector in the reverse (REV) and forward (FOR) orientations. Western analysis of 293T cells detects truncated C-terminal kinase domain of the BRAF protein (arrow) and full-length BRAF protein (arrowhead): Lane 1 tumor, Lane 2 GFP transfected, Lane 3 FOR, Lane 4 REV. (F and G) Expression of truncated BRAF results in foci formation in NIH 3T3 cells. NRAS (G12V) is an acutely transforming oncogene for comparison. Error bars indicate standard deviation.

FIG. 13. Analysis of double transgenic embryos and adults. (A) Structure of the T2/Onc2 transposon. (B) Transgenic transposon copy number estimates and percent methylated transposons determined following DraI/MspI or DraI/HpaII digestion. (C) Reduced number of E16 double transgenic embryos and adults. (D) Double transgenic embryos (left panel) were often smaller than control littermates. (E) 500 bp BamHI concatamer fragment (arrow) is reduced in intensity in double transgenic embryos and adults relative to T2/Onc2 heterozygous transgenic control. Adult tissues: brain DNA (odd numbered lanes) and kidney DNA (even numbered lanes).

FIG. 14. Generation and characterization of T2/Onc2 transgenic founders. (A) Tail biopsy DNA was digested with DraI, blotted and probed with a fragment of the En2 splice acceptor (underlined). The signal from the 1.5 kb transposon fragment was quantified by comparison to the 2.1 kb fragment from the En2 locus. (B) Tail biopsy DNA from transgenic animals was first digested with DraI then purified and cut with either MspI or HpaII. Genomic CpG methylation of the CCGG recognition sequence will inhibit HpaII but not MspI. The percentage of methylated sites can be determined by comparing the intensities of the 1.04 kb band detected by the probe (underlined) in the MspI and HpaII lanes for each DNA.

FIG. 15. Generation and characterization of RosaSB knock-in allele. (A) Structure of the wildtype and RosaSB knockin alleles [FRT sites, (triangles), SpeI sites (S), BamHI sites (B). (B) Southern blotting on tail biopsy DNA shows the predicted fragments and germline transmission of the RosaSB allele. Probe 1 was used on SpeI digested DNA, and probe 2 was used on BamHI digested DNA.

FIG. 16. Adult double transgenic mice die from cancer. (A) Survival curves show decreased viability of double transgenic mice. (B) Age at death and tumor type of double transgenic mice. (C) Southern analysis of BamHI-digested tumor DNA. Each band represents a separate SB transposon integration (SP=spleen, LN=lymph node, TH=thymus, M=mass).

FIG. 17. Medulloblastoma pathology. Hematoxilin and Eosin (H&E) stained sections of an SB-induced medulloblastoma and control cerebellum. (A) Section of the cerebellum from animal TG6057-17106 shows normal morphology with the Purkinje cell layer (P) adjacent to the granule cell layer (Gr). Tumor cells (T) have invaded the molecular layer (ML). (B) Comparable section for a normal cerebellum. (C) Tumor (T) has grown down brain stem adjacent to the spinal cord. (D) Comparable section for a normal spinal cord.

FIG. 18. Analysis of Notch1 integrations. (A) Structure of mutated Notch I allele in SB-induced T-cell leukemias. The exons are represented by the white squares and rectangles outside of the IRDR region, the transposon IRDRs are the central triangular elements, transposon splice acceptor is the left rectangle within the IRDR, the splice donor is the right rectangle within the IRDR, and the primer binding sites are shown as arrows (f=forward, r=reverse). (B) Northern analysis using a 3′ Notch1 cDNA probe showed that all tumors with Notch1 integrations (lanes 1-4) express a truncated Notch1 transcript. Transcript in tumor 16315 is less intense but can be seen on longer exposure. (C) RT-PCR shows that only tumors with Notch1 integration express a truncated Notch1 transcript.

FIG. 19. Notch1 cooperating genes. Clonality of Notch1, Rasgrp1, Sox8, and Runx2 integrations in Notch1 tumors was determined by Southern analysis (top). Quantitative PCR was used to measure the expression levels of Rasgrp1 (bottom left) and Runx2 (bottom right) in tumors relative to a Gapd control. Results are an average of three independent assays. Error bars represent the standard deviation. Error bars on the Runx2 graph are too small to visualize. Quantitative PCR could not be reliably performed on Sox8. Sox8 expression in tumors with Sox8 integrations was therefore monitored by RT-PCR.

FIG. 20. (A) An annotated map of the pCMV/SB plasmid and (B) a listing of the pCMV/SB plasmid nucleotide sequence (SEQ ID NO:8).

### DETAILED DESCRIPTION OF PREFERRED EMBODIMENTS OF THE INVENTION

Transposons

The present invention includes transposable elements, also referred to herein as “transposons.” Preferably, the transposon is able to excise from a donor polynucleotide, for instance, a vector, and integrate into a target site, for instance, a cell's genomic or extrachromosomal DNA. A transposon includes a polynucleotide that includes a nucleic acid sequence flanked by cis-acting nucleotide sequences on the termini of the transposon. In one aspect, the cis-acting nucleotide sequences are inverted repeats, as will be described herein.

As used herein, the term “polynucleotide” refers to a polymeric form of nucleotides of any length, either ribonucleotides or deoxynucleotides, and includes both double- and single-stranded DNA and RNA, and combinations thereof. A polynucleotide may include nucleotide sequences having different functions, including for instance coding sequences, and non-coding sequences such as regulatory sequences. A polynucleotide can be obtained directly from a natural source, or can be prepared with the aid of recombinant, enzymatic, or chemical techniques. A polynucleotide can be linear or circular in topology. A polynucleotide can be, for example, a portion of a vector, or a fragment.

As used herein, a “promoter” is a polynucleotide sequence that acts to assemble the subunits of RNA polymerase in a cell to initiate transcription of an operably linked downstream coding region, typically at a position 20 to 40 nucleotides downstream. An “enhancer” is a regulatory sequence that increases the rate of transcription initiation of a coding region. Enhancers usually exert their effect regardless of the distance, upstream or downstream location, or orientation of the enhancer relative to the start site of transcription. Without intending to be limiting, an enhancer is typically a nucleotide sequence where a polypeptide can bind and stabilize the association of RNA polymerase allowing initiation of transcription to proceed.

A “coding sequence” or a “coding region” is a polynucleotide that encodes a polypeptide and, when placed under the control of appropriate regulatory sequences, expresses the encoded polypeptide. The boundaries of a coding region are generally determined by a translational start codon at its 5′ end and a translational stop codon at its 3′ end. A coding region may include introns that are excised during RNA processing.

A regulatory sequence is a nucleotide sequence that regulates expression of a coding region to which it is operably linked. Non-limiting examples of regulatory sequences include promoters, transcriptional initiation sites, translational start sites, translational stop sites, transcriptional terminators (including, for instance, poly-adenylation signals), and intervening sequences (introns). “Operably linked” refers to a juxtaposition wherein the components so described are in a relationship permitting them to function in their intended manner. A regulatory sequence is “operably linked” to a coding region when it is joined in such a way that expression of the coding region is achieved under conditions compatible with the regulatory sequence.

As used herein, “polypeptide” refers to a polymer of amino acids and does not refer to a specific length of a polymer of amino acids. Thus, for example, the terms peptide, oligopeptide, protein, antibody, and enzyme are included within the definition of polypeptide. This term also includes post-expression modifications of the polypeptide, for example, glycosylations (e.g., the addition of a saccharide), acetylations, phosphorylations and the like.

An “isolated” polypeptide or polynucleotide means a polypeptide or polynucleotide that has been either removed from its natural environment, produced using recombinant techniques, or chemically or enzymatically synthesized. Preferably, a polypeptide or polynucleotide of this invention is purified, i.e., essentially free from any other polypeptide or polynucleotide and associated cellular products or other impurities.

A nucleic acid sequence is “flanked by” cis-acting nucleotide sequences if at least one cis-acting nucleotide sequence is positioned 5′ to the nucleic acid sequence, and at least one cis-acting nucleotide sequence is positioned 3′ to the nucleic acid sequence. A nucleic acid sequence flanked by cis-acting nucleotide sequences may be referred to herein as a “flanked sequence.” Cis-acting nucleotide sequences include at least one inverted repeat (also referred to herein as an inverted terminal repeat, or ITR) at each end of the transposon, to which a transposase, preferably a member of the Sleeping Beauty (SB) family of transposases, binds. The SB family of transposases is described in greater detail below.

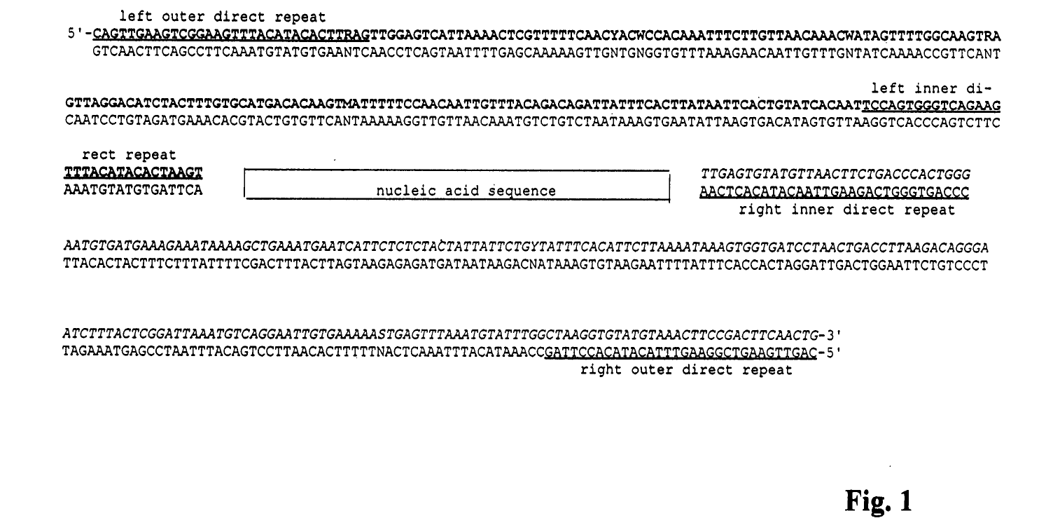

Each cis-acting inverted repeat that flanks a nucleic acid sequence preferably includes two or more direct repeats. A direct repeat is typically between about 25 and about 35 base pairs in length, preferably about 29 to about 31 base pairs in length. One direct repeat of an inverted repeat is referred to herein as an “outer repeat,” and is present at the end of the inverted repeat that is distal to the nucleic acid flanked by the inverted repeats. When a transposon excises from a donor polynucleotide (e.g., a vector) and integrates into a cell's genomic or extrachromosomal DNA, the outer repeats are juxtaposed to the cell's genomic or extrachromosomal DNA. The other direct repeat of an inverted repeat is referred herein as an “inner repeat,” and is present at the end of the inverted repeat that is proximal to the nucleic acid flanked by the inverted repeats. Thus, an inverted repeat on the 5′ or “left” side of a transposon of this embodiment typically comprises a direct repeat (i.e., a left outer repeat), an intervening region, and a second direct repeat (i.e., a left inner repeat). An inverted repeat on the 3′ or “right” side of a transposon of this embodiment comprises a direct repeat (i.e., a right inner repeat), an intervening region, and a second direct repeat (i.e., a right outer repeat) (see, for instance, FIG. 1). Further, an inverted repeat and the direct repeats within the inverted repeat on one side of a transposon are inverted with respect to the inverted repeat and the direct repeats within the inverted repeat on the other side of a transposon. Unless noted otherwise, the nucleotides of the inverted repeats as disclosed herein are on the same strand of DNA. It is understood that the complement of a left inverted repeat can be used on the right side of a transposon, and the complement of a right inverted repeat can be used on the left side of a transposon. Unless noted otherwise, the direct repeats are represented herein in a different manner: the nucleotide sequence of a direct repeat begins at the end of the inverted repeat that is distal to the nucleic acid flanked by the inverted repeats. Thus, a direct repeat present at the left side of a transposon is not on the same strand of DNA as a direct repeat present on the right side of a transposon (see FIG. 1).

The present invention is not limited to the use of a particular transposon element, and includes those described in, for instance Plasterk et al. (Trends Genet., 1999; 15:326-332), Plasterk et al. (U.S. Pat. No. 6,051,430), Kay et al. (U.S. Patent Application No. 2005/0003542), Kay et al. (WO 01/30965), Ivics et al. (WO 01/81565), Moran et al. (Cell, 1995; 87:917-927), Koga et al. (J. Hum. Genet., 2003; 48:231-235), and Miskey et al., (Nucl. Acids Res., 2003; 31:6873-6881). Preferably, the inverted repeats that bind SB transposase contain outer direct repeats that preferably have, in increasing order of preference, at least about 80% identity, at least about 90% identity, at least about 95% identity, most preferably, at least about 98% identity to a consensus direct repeat having the sequence 5′-CAGTTGAAGTCGGAAGTTTACATACACYTAAG (SEQ ID NO:3). Preferably, the inverted repeats that bind SB transposase contain inner direct repeats that preferably have, in increasing order of preference, at least about 80% identity, at least about 90% identity, at least about 95% identity, most preferably, at least about 98% identity to a consensus direct repeat having the sequence 5′-YCCAGTGGGTCAGAAGTTTACATACACTWART (SEQ ID NO:4). The nucleotide symbols used herein have the following meaning: R=G or A, Y=T or C, M=A or C, S=G or C, and W=A or T.

Nucleotide identity is defined in the context of a comparison between a direct repeat and SEQ ID NO:3 or SEQ ID NO:4, and is determined by aligning the residues of the two polynucleotides (i.e., the nucleotide sequence of the candidate direct repeat and the nucleotide sequence of SEQ ID NO:3 or SEQ ID NO:4) to optimize the number of identical nucleotides along the lengths of their sequences; gaps in either or both sequences are permitted in making the alignment in order to optimize the number of shared nucleotides, although the nucleotides in each sequence must nonetheless remain in their proper order. A candidate direct repeat is the direct repeat being compared to SEQ ID NO:3 or SEQ ID NO:4. Preferably, two nucleotide sequences are compared using the Blastn program of the BLAST 2 search algorithm, as described by Tatusova, et al. (FEMS Microbiol Lett., 1999; 174:247250), and available on the world wide web at the National Center for Biotechnology Information website, under BLAST in the Molecular Database section. Preferably, the default values for all BLAST 2 search parameters are used, including reward for match=1, penalty for mismatch=−2, open gap penalty=5, extension gap penalty=2, gap x dropoff=50, expect=10, wordsize=11, and optionally, filter on. In the comparison of two nucleotide sequences using the BLAST search algorithm, nucleotide identity is referred to as “identities.”

Examples of direct repeat sequences that bind to an SB transposase include: a left outer repeat 5′-CAGTTGAAGTCGGAAGTTTACATACACTTRAG (SEQ ID NO:22); a left inner direct repeat 5′-TCCAGTGGGTCAG AAGTTTACAT ACACTAAGT (SEQ ID NO:23); a right inner direct repeat 5′-CCCAGTGGGTCAGAAGTTAACATACACTCAA (SEQ ID NO:24) and a right outer repeat is 5′-CAGTTGAAGTCGGAAGTTTACATACACCTTAG (SEQ ID NO:25). Preferred examples of direct repeat sequences that bind to an SB transposase include: a left outer repeat 5′-CAGTTGAAGTCGGAAGTTTACATACACTTAAG-3′ (SEQ ID NO:13); left inner repeats 5′-TCCAGTGGGTCAGAAGTTTACATACACTAAGT-3′ (SEQ ID NO:14) and 5′-TCCAGTGGGTCAGAAGTTTACATACACTTAAG-3′ (SEQ ID NO:15); right inner repeats 5′-CCCAGTGGGTCAGAAGTTTACATACACTCAAT-3′ (SEQ ID NO: 16); and a right outer repeat 5′-CAGTTGAAGTCGGAAGTTTACATACACCTTAG-3′ (SEQ ID NO:17).

In one embodiment the direct repeat sequence includes at least 5′-TCRGAAGTTTACATACAC (SEQ ID NO:34), more preferably 5′-GTCRGAAGTTTACATACAC (SEQ ID NO:29).

The intervening region within an inverted repeat is generally at least about 150 base pairs in length, preferably at least about 160 base pairs in length. The intervening region is preferably no greater than about 200 base pairs in length, more preferably no greater than about 180 base pairs in length. In a transposon, the nucleotide sequence of the intervening region of one inverted repeat may or may not be similar to the nucleotide sequence of an intervening region in another inverted repeat.

Preferably, the inverted repeats that bind SB transposase contain intervening regions that preferably have, in increasing order of preference, at least about 80% identity, at least about 90% identity, at least about 95% identity, most preferably, at least about 98% identity to SEQ ID NO:30, or the complement thereof.

Preferred examples of intervening regions include

SEQ ID NO:30

5′ TTGGAGTCAT TAAAACTCGT TTTTCAACYA CWCCACAAAT
TTCTTGTTAA CAAACWATAG TTTTGGCAAG TCRGTTAGGA
CATCTACTTT GTGCATGACA CAAGTMATTT TTCCAACAAT
TGTTTACAGA CAGATTATTT CACTTATAAT TCACTGTATC
ACAAT 3′,

and the complement thereof,

SEQ ID NO:31

5′ AATGTGATGA AAGAAATAAA AGCTGAAATG AATCATTCTC
TCTACTATTA TTCTGAYATT TCACATTCTT AAAATAAAGT
GGTGATCCTA ACTGACCTTA AGACAGGGAA TCTTTACTCG
GATTAAATGT CAGGAATTGT GAAAAASTGA GTTTAAATGT
ATTTGG- 3′,

and the complement thereof,

SEQ ID NO:32

5′ AATGTGATGA AAGAAATAAA AGCTGAAATG AATCATTCTC
TCTACTATTA TTCTGAYATT TCACATTCTT AAAATAAAGT
GGTGATCCTA ACTGACCTAA GACAGGGAAT TTTTACTAGG
ATTAAATGTC AGGAATTGTG AAAASGTGAG TTTAAATGTA
TTTGG- 3′,

and the complement thereof. and

Preferably, inverted repeats that bind SB transposase have, in increasing order of preference, at least about 80% identity, at least about 90% identity, at least about 95% identity, most preferably, at least about 98% identity to SEQ ID NO:1, or the complement thereof. Nucleotide identity is determined as described hereinabove.

One preferred left inverted repeat sequence of this invention is

SEQ ID NO:6

5′ CAGTTGAAGT CGGAAGTTTA CATACACTTA RGTTGGAGTC
ATTAAAACTC GTTTTTCAAC YACWCCACAA ATTTCTTGTT
AACAAACWAT AGTTTTGGCA AGCRAGTTAG GACATCTACT
TTGTGCATGA CACAAGTMAT TTTTCCAACA ATTGTTTACA
GACAGATTAT TTCACTTATA ATTCACTGTA TCACAATTCC
AGTGGGTCAG AAGTTTACAT ACACTAAGT- 3′,

and the complement thereof, and another preferred inverted repeat sequence of this invention is

SEQ ID NO:7

5′ TTGAGTGTAT GTTAACTTCT GACCCACTGG GAATGTGATG
AAAGAAATAA AAGCTGAAAT GAATCATTCT CTCTACTATT
ATTCTGAYAT TTCACATTCT TAAAATAAAG TGGTGATCCT
AACTGACCTT AAGACAGCGA ATCTTTACTC GGATTAAATG
TCACGAATTG TGAAAAASTG AGTTTAAATG TATTTGGCTA
AGGTGTATGT AAACTTCCGA CTTCAACTG- 3′,

and the complement thereof.

The inverted repeat (SEQ ID NO:7) contains the poly(A) signals AATAAA at nucleotides 46-51 and 104-109. These poly(A) signals can be used by a coding sequence present in the transposon to result in addition of a poly(A) tail to an mRNA. The addition of a poly(A) tail to an mRNA typically results in increased stability of that mRNA relative to the same mRNA without the poly(A) tail.

A more preferred inverted repeat sequence of this invention is

SEQ ID NO:1

5′ CAGTTGAAGT CGGAAGTTTA CATACACTTA AGTTGGAGTC
ATTAAAACTC GTTTTTCAAC TACTCCACAA ATTTCTTGTT
AACAAACAAT AGTTTTGGCA AGTCAGTTAG GACATCTACT
TTGTGCATGA CACAAGTCAT TTTTCCAACA ATTGTTTACA
GACAGATTAT TTCACTTATA ATTCACTGTA TCACAATTCC
AGTGGGTCAG AAGTTTACAT ACACTAAGT- 3′,

and the complement thereof.

Another more preferred inverted repeat sequence of this invention is

SEQ ID NO:2

5′ ATTGAGTGTA TGTAAACTTC TGACCCACTG GGAATGTGAT
GAAAGAAATA AAAGCTGAAA TGAATCATTC TCTCTACTAT
TATTCTGAYA TTTCACATTC TTAAAATAAA GTGGTGATCC
TAACTGACCT AAGACAGGGA ATTTTTACTA GGATTAAATG
TCAGGAATTC TGAAAASGTG AGTTTAAATG TATTTGGCTA
AGGTGTATGT AAACTTCCGA CTTCAACTG- 3′,

and the complement thereof.

Yet another more preferred left inverted repeat sequence of this invention is

SEQ ID NO:33

5′ CAGTTGAAGT CGGAAGTTTA CATACACGGG GTTTGGAGTC
ATTAAAACTC GTTTTTCAAC TACTCCACAA ATTTCTTGTT
AACAAACAAT AGTTTTGGCA AGTCAGTTAG GACATCTACT
TTGTGCATGA CACAAGTCAT TTTTCCAACA ATTGTTTACA
GACAGATTAT TTCACTTATA ATTCACTGTA TCACAATTCC
AGTGGGTCAG AAGTTTACAT ACACTAAGT- 3′,

and the complement thereof.

In some preferred aspects of the present invention, a transposon includes SEQ ID NO: 1 as the left inverted repeat and SEQ ID NO:2 as the right inverted repeat, or the complement of SEQ ID NO:2 as the left inverted repeat and the complement of SEQ ID NO:1 as the right inverted repeat. In another preferred aspect, a transposon includes SEQ ID NO:33 as the left inverted repeat and the complement of SEQ ID NO:33 as the right inverted repeat.

A transposon of the present invention is able to excise from a donor polynucleotide (for instance, a vector) and integrate into a cell's genomic or extrachromosomal DNA. Assays for measuring the excision of a transposon from a vector, the integration of a transposon into the genomic or extrachromosomal DNA of a cell, and the ability of transposase to bind to an inverted repeat are described herein and are known to the art (see, for instance, Ivics et al., Cell, 1997; 91:501-510; WO 98/40510 (Hackett et al.); WO 99/25817 (Hackett et al.), WO 00/68399 (Mclvor et al.), and U.S. application Ser. No. 10/128,998 (Steer et al.). For an assay that can be used to measure the level of transposition, see Example 3, herein. Preferably, the level of transposition is high enough to provide a sufficient level of non-local hopping to reach genes on chromosomes beyond the chromosome on which excision occurred.

A transposon of the present invention may be present in a variety of locations. For instance, a transposon of the invention may be present in the genomic DNA of a chromosome of a cell. A transposon of the present invention may also be present in a vector. A vector is a replicating polynucleotide, such as a plasmid, to which another polynucleotide may be attached so as to bring about the replication of the attached polynucleotide. The vector may include a coding sequence. A vector can provide for further cloning (amplification of the polynucleotide), i.e., a cloning vector, or for expression of the polypeptide encoded by the coding region, i.e., an expression vector. A vector can be both a cloning vector and an expression vector. The term vector includes, but is not limited to, plasmid vectors, cosmid vectors, artificial chromosome vectors, or, in some aspects of the invention, viral vectors. Examples of viral vectors include adenovirus, herpes simplex virus (HSV), alphavirus, simian virus 40, picomavirus, vaccinia virus, retrovirus, lentivirus, and adeno-associated virus. Preferably the vector is a plasmid. In some aspects of the invention, a vector is capable of replication in the cell to which it is introduced; in other aspects the vector is not capable of replication. In some preferred aspects of the present invention, the vector is unable to mediate the integration of the vector sequences into the genomic or extrachromosomal DNA of a cell. An example of a vector that can mediate the integration of the vector sequences into the genomic or extrachromosomal DNA of a cell is a retroviral vector, in which the integrase mediates integration of the retroviral vector sequences.

Preferably, the vector includes specific nucleotide sequences that are juxtaposed to the transposon. For instance, a vector includes a “TAACCC” on one the right side of the transposon and a “GGGGA” on the left side of the transposon, or an “AAATA” on the right side of the transposon and a “TGTCT” on the left side of the transposon, or a “TTGAT” on one the right side of the transposon and a “CTCGG” on the left side of the transposon, or a “TGCCT” on one the right side of the transposon and a “ACGTA” on the left side of the transposon. More preferably, the vector includes specific nucleotide sequences which are juxtaposed to the transposon, and increase the frequency of transposition of the transposon compared to the frequency of transposition of the transposon when the vector includes, for instance, a “TAACCC” on one the right side of the transposon and a “GGGGA” on the left side of the transposon. For instance, a vector more preferably includes a “TATA” nucleotide sequence that is present the left side of the transposon, or an “ATAT” on the right side of the transposon. Even more preferably, the vector includes a “TATA” nucleotide sequence that is present on the left side, and an “ATAT” on the right side of the transposon. Alternatively, the vector may include a “TGATA” on the right side of the transposon and a “CTGTA” on the left side of the transposon. Preferably, the vector does not include a “TTAAG” on one the right side of the transposon and an “AATAA” on the left side of the transposon, or an “AACTA” on one the right side of the transposon and a “TGGCT” on the left side of the transposon, or an “AGCCA” on one the right side of the transposon and a “TAGTT” on the left side of the transposon. Construction of vectors containing a polynucleotide of the invention employs standard ligation techniques known in the art. See, e.g., Sambrook et al, Molecular Cloning: A Laboratory Manual, Cold Spring Harbor Laboratory Press (1989) or Ausubel, R. M., ed. Current Protocols in Molecular Biology (1994).

Preferably, delivery of the transposon to the DNA of a cell, using a vector as described above or through other delivery methods known to those skilled in the art, results in the presence of a concatamer of transposons. A concatamer, as defined herein, is an end-to-end array of a plurality of identical nucleotide sequences. A concatamer of transposons thus provides a series of multiple transposons encoded in a long sequence with the DNA of a cell. The formation of a concatamer of transposons is advantageous in the methods of use detailed herein as transposons of the present invention are not copied during transposition when a cut and paste mechanism, such as that used by the SB transposase, is being used. Thus, the initial transposons are a finite resource from which transposons are mobilized to different locations. By providing a large number of transposons, a concatamer serves as a richer source of transposons, leading to a larger number of transposon insertions throughout the DNA of the cell.

Polynucleotides of the present invention include a nucleic acid sequence flanked by cis-acting nucleotide sequences. The nucleic acid sequence is often referred to herein as a “flanked sequence.” The cis-acting nucleotide sequences include at least one inverted repeat at each end of the transposon, as described herein. The flanked sequence includes one or more nucleic acid sequences that act as insertional mutagens. An insertional mutagen is a nucleic acid sequence whose insertion will affect the level of expression or the nature of the product expressed by a coding region near or in which the flanked sequence is inserted by transposition. When the nature of the product expressed is altered, the nucleic acid is referred to as a “disruptive sequence.” When the level of expression is altered, the nucleic acid is referred to as an “affective sequence.” Transposons of the present invention may include one or more insertional mutagens, which may be disruptive and/or affective sequences.

In one aspect of the invention, the flanked sequence includes a non-coding sequence that can alter the nature of the product expressed by a coding region when the transposon inserts the nucleotide sequence in or near that coding region in a cell, referred to herein as “a disruptive sequence.” Any nucleotide sequence that will alter the nature of the product expressed by a coding region present in the cell can be used. Examples of disruptive sequences include multiple stop codons in each of the possible frames, transcription terminators, splice acceptor sites, splice donor sites, and silencer elements. A disruptive sequence may, for example, lead to the formation of a truncated protein during protein expression. An example of a truncated protein is provided in Example 1, herein, which describes a truncated BRAF protein expressed in sarcomas, with T2/Onc integrations in the ninth intron of Braf, that contains only the kinase domain and lacks the N-terminal negative regulatory elements of the protein.

In some aspects, the disruptive sequence includes a splice acceptor site. A splice acceptor site is a nucleotide sequence that is generally involved in RNA splicing to remove intronic RNA sequences. While not intending to be bound by theory, the splice acceptor site is normally involved in the excision of introns, during which it is bound by an RNA-protein complex referred to as a spliceosome, cleaved, and then joined to a splice donor site that has already been cleaved, resulting in the excision of an intervening portion of the nucleotide sequence in a lariat formation. Splice acceptor sequences are well known in the art, and can be readily obtained from genes at a position between the exon and intron where they mediate splicing. Alternately, SA sites may be chemically or enzymatically synthesized. Whether a polynucleotide functions as a splice acceptor can be easily determined using methods known in the art. Splice Acceptor (SA) sites typically end in AG dinucleotides that are highly conserved. The remaining nucleotides of the sequence are primarily cytidine or thymidine. Exemplary SA sites include the nucleotide sequences SEQ ID NO:9 5′ CCCCCCCCCCCNCAG-3′ and SEQ ID NO:10 5′ TTTTTTTTTTTNTAG-3′, where N represents a nucleotide that can be either A, G, C, or T. For example, an SA site used in an embodiment of the invention is nucleotides 533 to 630 of SEQ ID NO:19. A preferred SA site is the engrailed-2 (En2) SA, disclosed by the complement of nucleotides 1686 to 1959 of the pT2/Onc2 sequence, SEQ ID NO:19. En2 is a well-characterized splice acceptor used for gene trap mutagenesis in mouse embryonic cells (Genes Dev., 1992 June; 6(6):903-18). In a further aspect, the flanked sequence includes two splice acceptor sites, with the second splice acceptor site being provided in an orientation opposite that of the first splice acceptor site. This allows a splice acceptor site to be properly read during transcription regardless of the orientation of the transposon. Splice acceptor sites, as well as splice donor sites, are described in further detail by Padgett et al, Ann. Rev. Biochem. J., 1988; 55:1119-1150.

In an additional aspect of the invention, the flanked sequence including a splice acceptor site also includes a transcription termination signal site operably linked to the SA site. A termination signal site may be, for example, a polyadenylation (pA) signal site. If there are two SA sites, this may result in the pA signal sites being positioned between the two SA sites, due to the opposite orientation of the SA sites. In one aspect of the invention, the two pA sites, positioned between the two SA sites, may be replaced by a single, bidirectional pA site. For example, a bidirectional pA site is disclosed by the complement of nucleotides 1444 to 1525 of SEQ ID NO:19. Polyadenylation signal sites are well known by those skilled in the art, and can be readily obtained from genes where they are used to terminate transcription, or can be chemically or enzymatically synthesized. Whether a polynucleotide functions as a polyadenation signal can be easily determined using methods known in the art. While not intending to be bound by theory, the pA signal site provides a signal to polyadenylate a cleavage site that typically occurs about 15-30 nucleotides downstream from the pA signal site. Polyadenylation generally results in the addition of about 200 adenylate (AMP) residues to form a poly(A) tail on the mRNA formed. A polyadenylation (pA) signal site preferably includes the nucleotide sequence AAUAAA. The provision of two SA sites with downstream pA sites facilitates gene trapping that can terminate transcription when integrated in either orientation in a gene when the flanked sequence is inserted downstream from a coding sequence, as splicing will occur during transcription between the SA site of the flanked sequence and the SD site of an upstream gene.

In a further aspect of the invention, the disruptive sequence includes a splice donor. Splice donors are described in further detail, herein. Splice donors may also result in truncation of an expressed protein by insertion within an intron region within a protein. For instance, in Example 1 below, truncation of the Braf gene involved disruption facilitated by splice donor regions.

In another aspect of the invention, the flanked sequence includes a non-coding sequence that can alter the level of expression of a coding region when the transposon inserts near that coding region in a cell, referred to herein as “affective sequences.” The affective sequence may either increase or decrease the level of expression of a coding region; preferably to increase the level of expression of a coding region. Any nucleotide sequence that will alter the level of expression of a coding region present in a cell can be used. Examples of affective sequences include enhancers, promoters, matrix attachment sequences, and transcription binding sites. Enhancers and promoters have been defined herein. Matrix-attached regions (MARs) have been demonstrated to nest origins of replication and transcriptional enhancers, and rules have been proposed to facilitate the classification of a DNA sequence as a matrix attachment region (Boulikas, J. Cell Biochem., 1993; 52(1): 14-22). A transcription binding site is a nucleotide region with an affinity for transcription factors that alters the expression of a coding region upon binding by a transcription factor. Transcription binding sites are generally found within promoters or enhancers. Transcription factors include, for example, homeodomain proteins, POU transcription factors, DNA bending proteins, and zinc finger transcription factors. Useful promoters, enhancers, and transcription binding sites are readily available and known to those skilled in the art. Affective sequences may, for example, lead to the increased expression of a signal transduction protein produced by a coding region.

A preferred affective sequence for use in the invention is a promoter. Various promoters are readily available to the skilled person and are used routinely. Useful promoters include constitutive promoters, tissue specific promoters, and developmental stage specific promoters. For example, promoters include the human cytomegalovirus immediate early promoter and the EF1α promoter, which are constitutive promoters, and the rat probascin 1 promoter and the Pax2 promoter, which are active in the prostate gland and the developing hindbrain, eye, and urogenital system, respectively. Preferably, the promoter is a strong promoter; e.g., it is able to cause a significant increase in expression of an operably linked coding region. Useful promoters include promoters that function in many different types of cells, for instance, lung cells, gastrointestinal tract cells, and brain cells. More preferably, this promoter sequence is a long terminal repeat (LTR) sequence. LTR sequences are preferred as they are strong and ubiquitous, and have been shown to be capable of activating oncogenes upon insertion. A particularly preferred LTR is the LTR of the Murine stem cell virus (MSCV). An example of an MSCV LTR is disclosed by nucleotides 807 to 1207 of SEQ ID NO:19. LTRs are retroviral transcriptional control sequences that contain identical sequences that can be divided into three elements; U3, R, and U5. The U3 region of an LTR typically includes both a promoter and an enhancer. Further information on LTRs may be found in Retroviruses, eds. Coffin et al., p. 205-261 (1997).

In an additional aspect of the invention, the flanked sequence including an affective sequence also includes a splice donor (SD) site operably linked to the affective sequence. A splice donor site is a nucleotide sequence that is generally involved in RNA splicing to remove intronic RNA sequences. While not intending to be bound by theory, the splice donor site typically is cleaved by nucleophilic attack at the 5′ splice junction and is then bound to a splice acceptor site after cleavage at the 3′ splice junction. The splicing mechanism that removes an intron utilizing the splice donor and splice acceptor sites is mediated by a spliceosome. Splice Donor (SD) sites typically end in GT (or GU) dinucleotides that are highly conserved. Splice donor sequences are well known in the art, and can be readily obtained from genes at a position between the exon and intron where they mediate splicing. Alternately, SD sites may be chemically or enzymatically synthesized. Whether a polynucleotide functions as a splice donor can be easily determined using methods known in the art. Preferred SD sites include the nucleotide sequences GTAAGT and GTGAGT. An example of an SD site is disclosed at nucleotides 1217 to 1394 of SEQ ID NO:19. If two splice acceptor sites are provided, the splice donor site is preferably positioned between the two SA sites. This results in the upstream SA being in the improper orientation, thus avoiding mere excision of a portion of the flanked sequence.

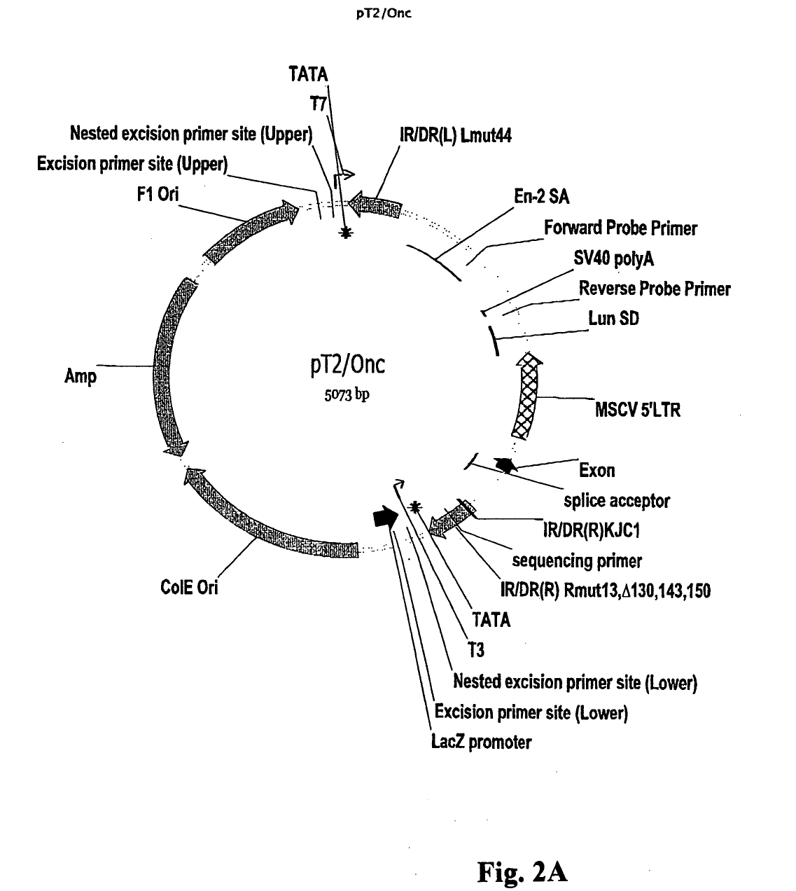

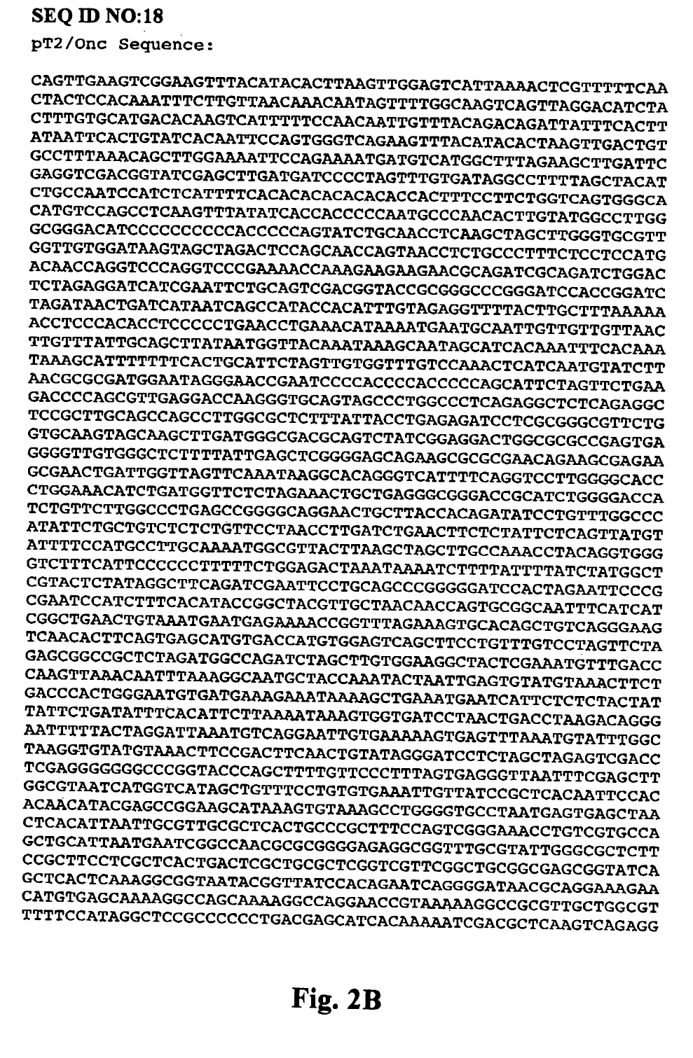

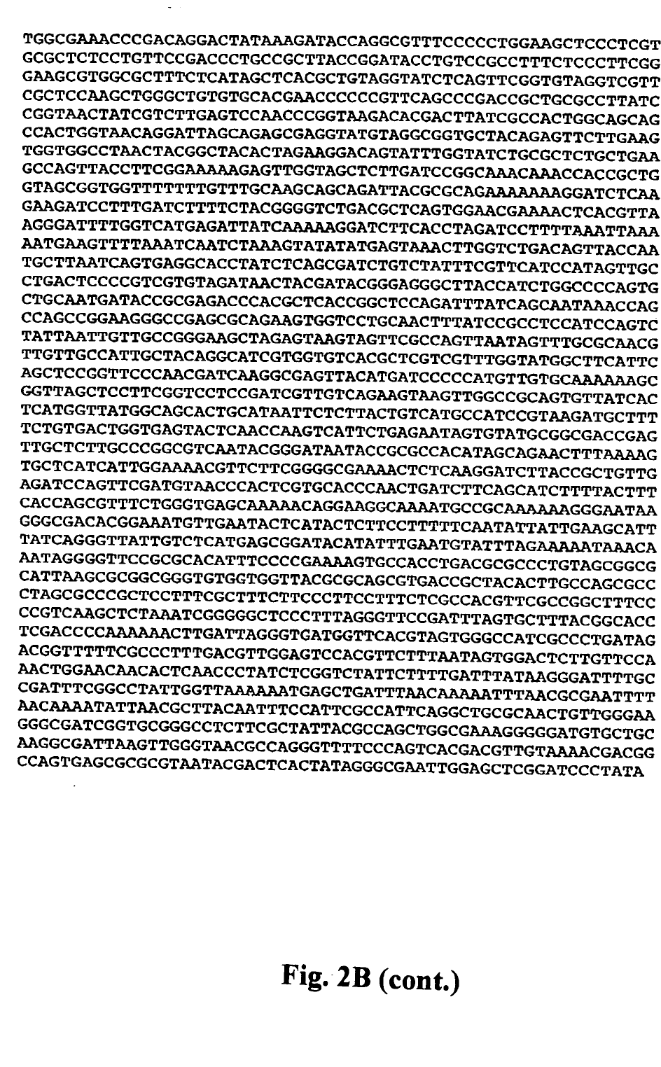

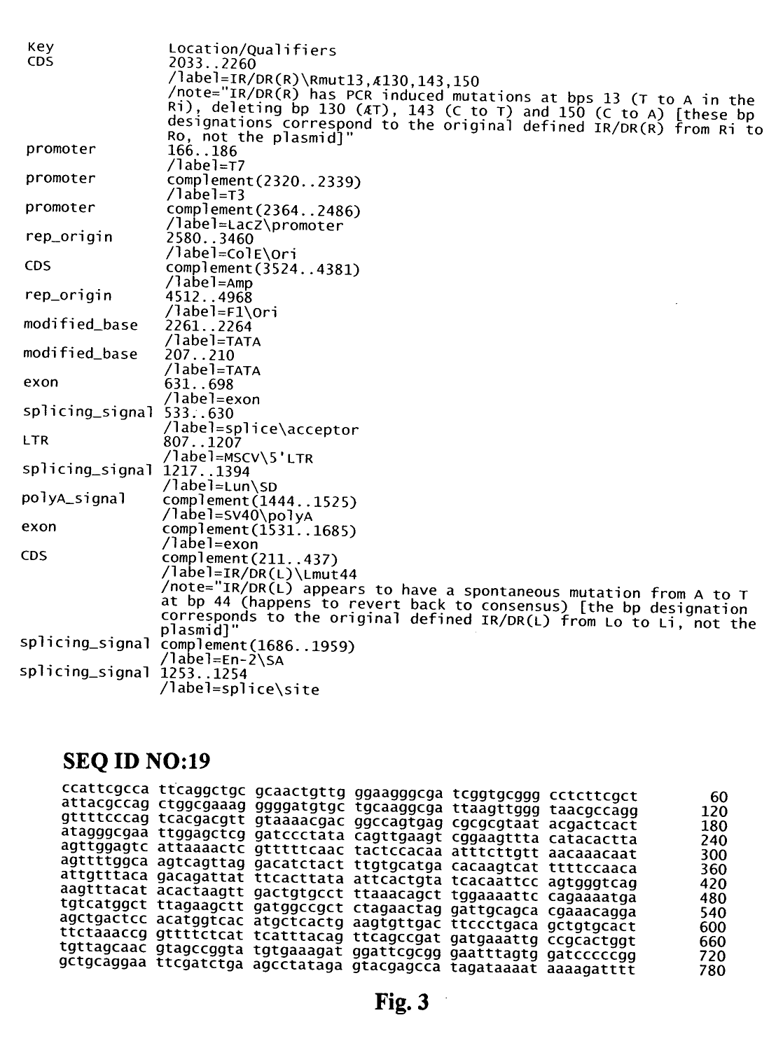

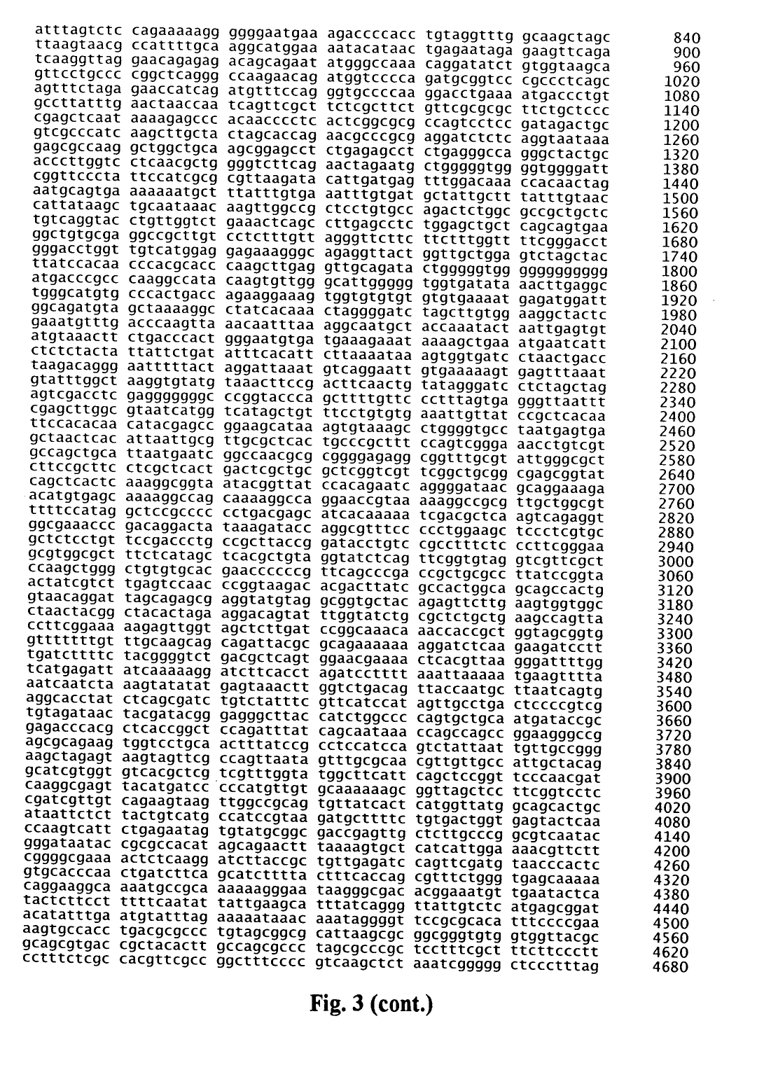

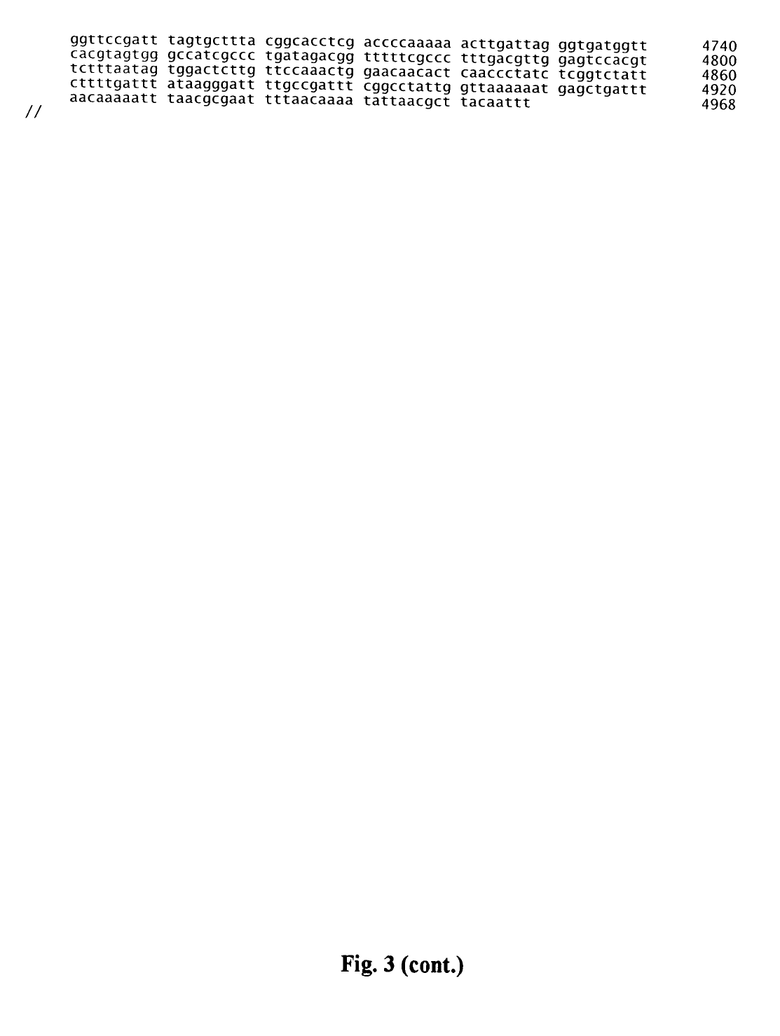

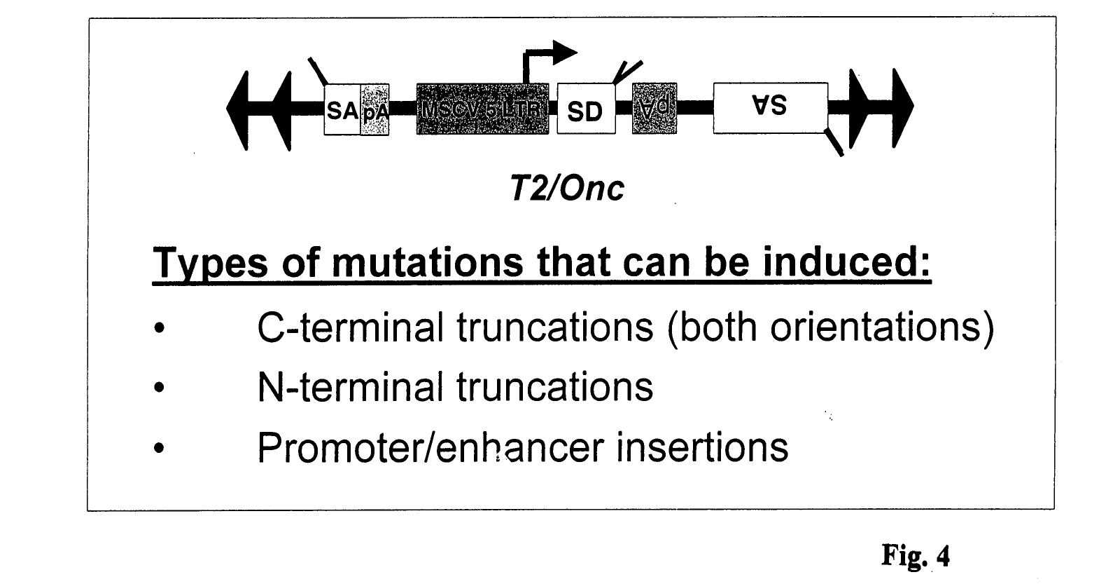

A transposon of the present invention may include one or more disruptive sequences and one or more affective sequences, or a combination thereof. A preferred embodiment of the invention is shown in FIG. 2A, which provides an annotated map of a plasmid containing the T2/Onc transposon. The nucleotide sequence of the plasmid containing the T2/Onc transposon (SEQ ID NO: 18) is provided in FIG. 2B. The T2/Onc transposon contains, going from the 5′ to the 3′ end, an IR/DR(L) sequence, a first SA site, an MSCV LTR, an SD site, and (in inverted orientation), a pA site and a second SA, flanked at the end by an IR/DR(R) sequence, marking the end of the transposon. The second SA site contains a larger fragment of the engrailed-2 (En2) SA. An alternate T2/Onc2 transposon is also shown in FIG. 3A, with its sequence (SEQ ID NO:19) shown in FIG. 3B. This transposon is similar to that of SEQ ID NO:18, but contains a larger fragment of the engrailed-2 (En2) splice acceptor (SA) and is flanked by optimized SB transposase binding sites that increase SB transposition. The ITRs used are SB transposase binding sites that increase SB transposition, as described above. A pictorial version of the T2/Onc transposon (SEQ ID NO: 18) is shown in FIG. 4, which highlights the insertional mutagen elements within the flanked region of the transposon in one embodiment of the invention. The transposon in this embodiment may be smaller than other SB transposons used previously (˜2.0 kb), in order to approach the optimal transposon size for transposition.

A coding sequence may also be present in the flanked sequence that encodes a polypeptide that permits the cell containing the polypeptide to be detected. Selectable markers permit the selection of cells containing the selectable marker. An example of a type of selectable marker is drug resistance, including, for instance, resistance to the neomycin analog G418. Detectable markers may permit identification of cells containing the detectable marker. Examples of such detectable markers that can be used in this way include fluorescent proteins (e.g., green, yellow, blue, or red fluorescent proteins), luciferase, and chloramphenicol acetyl transferase, β-galactosidase, and other molecules detectable by their fluorescence, enzymatic activity or immunological properties, and are typically useful when detected in a cell, for instance, a cultured cell, or a tissue sample that has been removed from an animal. Detectable markers also include markers that are secreted by cells to allow identification of an animal that contains a cell containing the detectable marker, for instance, secreted alkaline phosphatase, and α-1-antitrypsin.

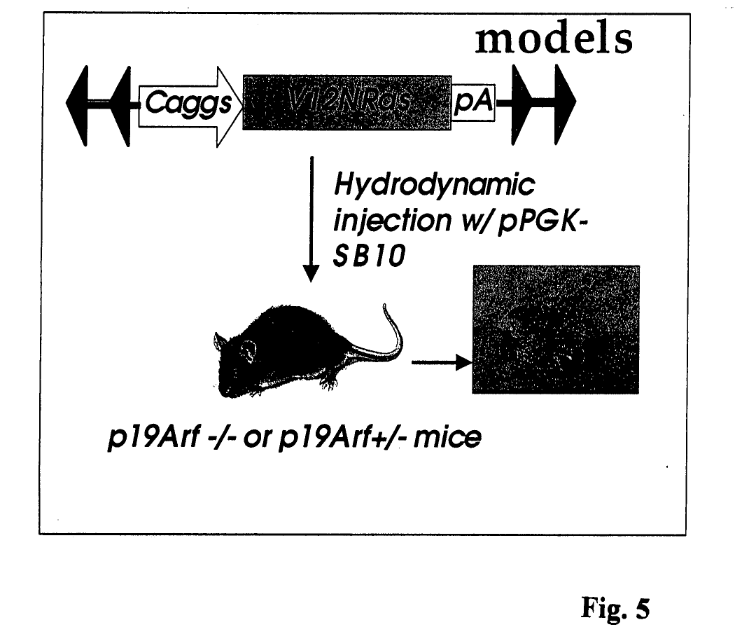

A coding sequence present on a transposon of the present invention may also encode an oncogene. Direct provision of an oncogene provides a useful addition or alternative to formation of oncogenes using insertional mutagens, as described herein. Preferably, an oncogene provided within a transposon is provided with a promoter that is operably linked to the oncogene. Furthermore, it is preferable to provide the oncogene with a pA signal sequence. Any oncogene known by those skilled in the art can be inserted using the transposon system of the present invention. Example genes that provide oncogenes include erbB-2, Ras, Src, Bcl-2, and telomerase-encoding genes. Oncogenes and promoters that can be operably linked to the oncogenes are readily available, and are known to those skilled in the art. An example of a transposon that provides an oncogene is shown in FIG. 5, which shows an NRAS(V12) expressing SB transposon that includes a CAGGS promoter and a pA signal can be used to induce multifocal cholangiocarcinoma or myeloproliferative disease and sarcoma in mice.

Transposases

The present invention is not limited to the use of a particular transposase, provided the transposase mediates transposition of the transposon. Preferably, the transposase binds an inverted sequence of the present invention or a direct repeat of the present invention, and preferably catalyzes the excision of a transposon from a donor polynucleotide (e.g., a vector) and subsequent integration of the transposon into the genomic or extrachromosomal DNA of a target cell. The transposase may be present as a polypeptide. Alternatively, the transposase is present as a polynucleotide that includes a coding sequence encoding a transposase. The polynucleotide can be RNA, for instance an mRNA encoding the transposase, or DNA, for instance a coding sequence encoding the transposase. The polynucleotide encoding a transposase may be on a vector, or present in a chromosome. When the transposase is present as a coding sequence encoding the transposase, in some aspects of the invention the coding sequence may be present on the same polynucleotode (e.g., a vector) that includes the transposon, i.e., in cis. In other aspects of the invention, the transposase coding sequence may be present on a second polynucleotide (e.g., a vector), i.e., in trans.

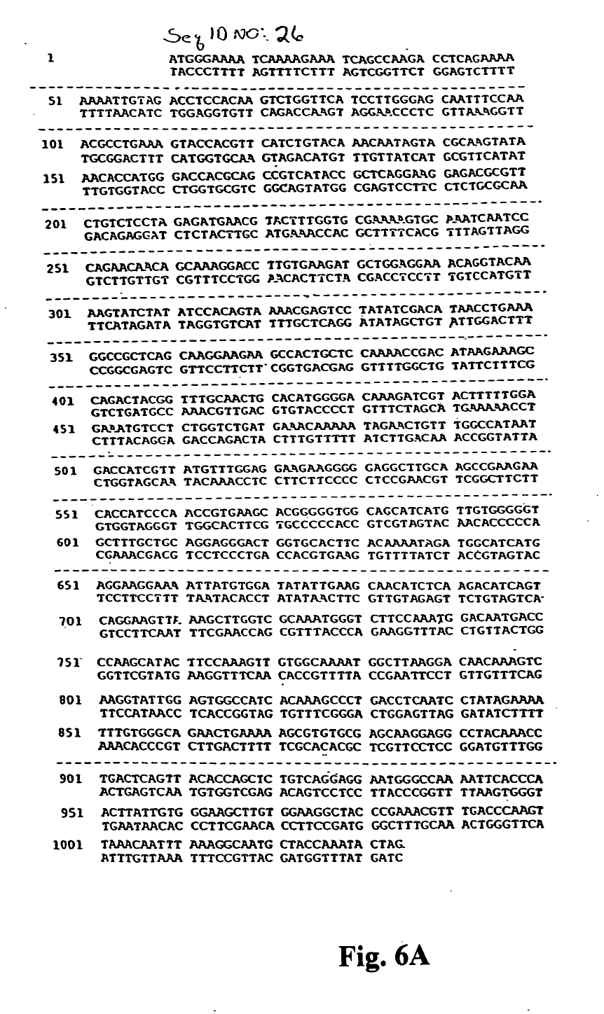

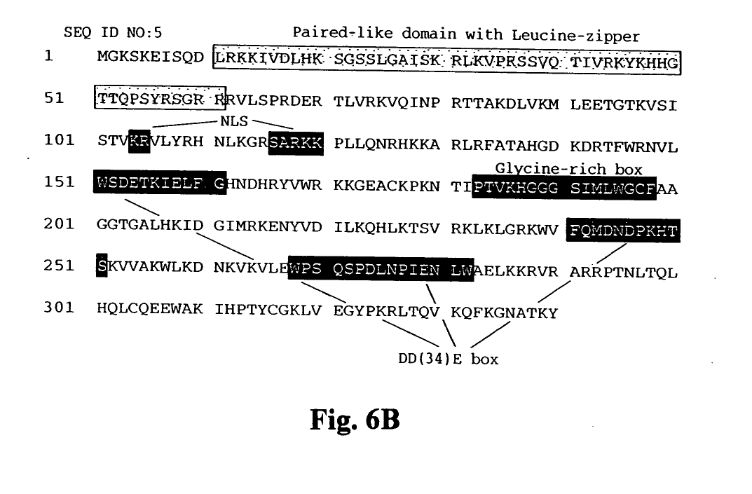

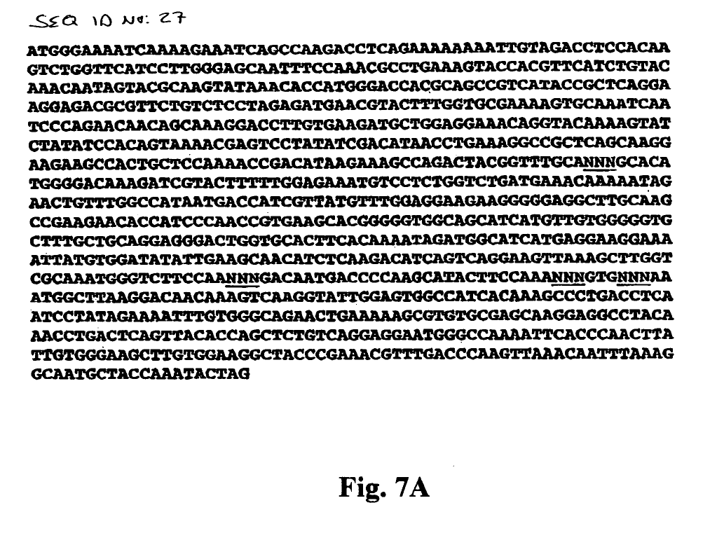

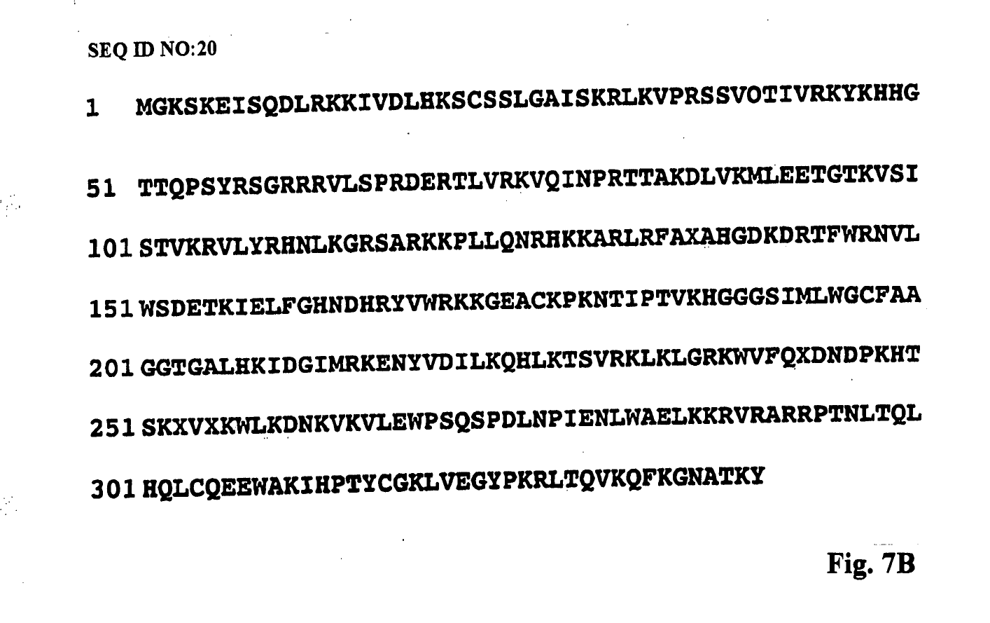

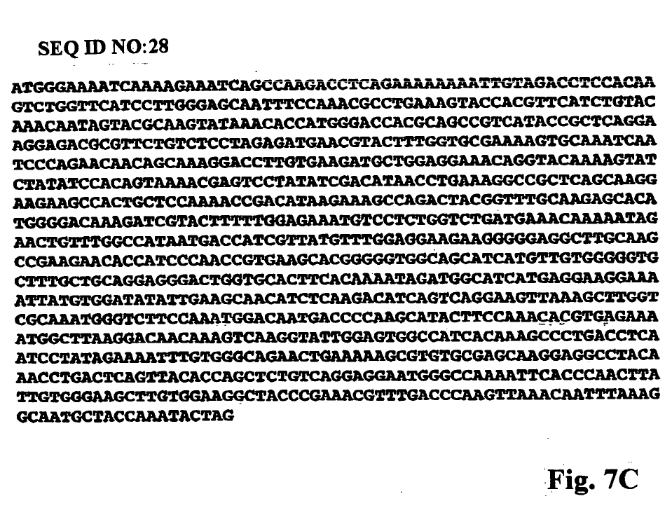

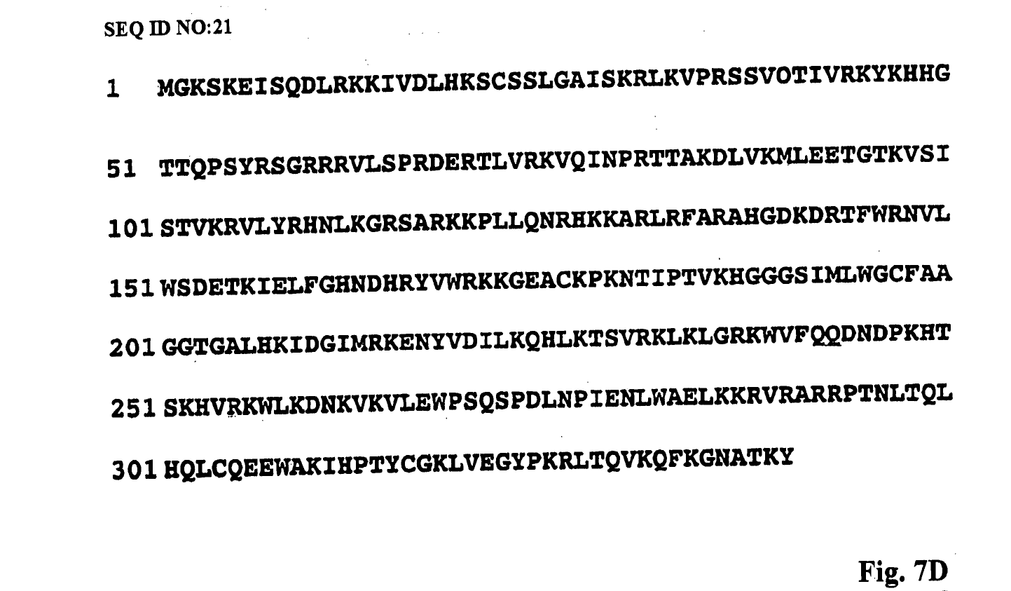

A preferred transposase for use in the invention is “Sleeping Beauty” transposase, referred to herein as SB transposase (Ivics et al. Cell, 1997; 91:501-510); WO 98/40510 (Hackett et al.); WO 99/25817 (Hackett et al.), WO 00/68399 (Mclvor et al.), U.S. Appl. No. 2005/0003542 (Kay et al.). SB transposase is able to bind the inverted repeat sequences of SEQ ID NOs:6-7 and direct repeat sequences (SEQ ID NOs:13-17) from a transposon, as well as a consensus direct repeat sequence (SEQ ID NO:3 or SEQ ID NO:4). SB transposase includes, from the amino-terminus moving to the carboxy-terminus, a DNA-binding domain, nuclear localizing domains (NLS) domains and a catalytic domain including a DD(34)E box and a glycine-rich box, as described in WO 98/40510 (Hackett et al.). The SB family of polypeptides includes the polypeptide having the amino acid sequence of SEQ ID NO:5 (FIG. 6A), SEQ ID NO:20 (FIG. 7A), and SEQ ID NO:21 (FIG. 7C), and the polypeptides described in WO 01/81565 (Ivics et al.).

Preferably, a member of the SB family of polypeptides also includes polypeptides with an amino acid sequence that shares at least about 80% amino acid identity to SEQ ID NO:21, more preferably, it shares at least about 90% amino acid identity therewith, most preferably, about 95% amino acid identity. Amino acid identity is defined in the context of a comparison between the member of the SB family of polypeptides and SEQ ID NO:21, and is determined by aligning the residues of the two amino acid sequences (i.e., a candidate amino acid sequence and the amino acid sequence of SEQ ID NO:21) to optimize the number of identical amino acids along the lengths of their sequences; gaps in either or both sequences are permitted in making the alignment in order to optimize the number of identical amino acids, although the amino acids in each sequence must nonetheless remain in their proper order. A candidate amino acid sequence is the amino acid sequence being compared to an amino acid sequence present in SEQ ID NO:21. A candidate amino acid sequence can be isolated from a natural source, or can be produced using recombinant techniques, or chemically or enzymatically synthesized. Preferably, two amino acid sequences are compared using the Blastp program, version 2.2.10, of the BLAST 2 search algorithm, as described by Tatusova et al. (FEMS Microbiol. Lett., 1999; 174:247-250), and available on the world wide web at the National Center for Biotechnology Information website, under BLAST in the Molecular Database section. Preferably, the default values for all BLAST 2 search parameters are used, including matrix=BLOSUM62; open gap penalty=11, extension gap penalty=1, gap x_dropoff=50, expect=10, wordsize=3, and optionally, filter on. In the comparison of two amino acid sequences using the BLAST search algorithm, structural similarity is referred to as “identity.” SB transposases preferably have a molecular weight range of about 35 kDa to about 40 kDa on about a 10% SDS polyacrylamide gel. An SB transposase must further retain activity as a transposase such that it can catalyze the excision of an SB transposon and integration into a target site.

Nucleic acid sequences encoding the SB transposases of SEQ ID NO: 5 (SB 10 transposase) and SEQ ID NO: 21 (SB11 transposase) are known. For example, SEQ ID NO: 26 is a representative nucleic acid sequence that encodes SB10 transposase, and is shown in FIG. 6B. SEQ ID NO:28, on the other hand, is a representative nucleic acid sequence that encodes SB11 transposase. It will further be understood by those skilled in the art that owing to the degeneracy of the genetic code, a sizeable yet definite number of DNA sequences can be constructed to encode peptides having an amino acid sequence corresponding to a transposase.

The coding region encoding a transposase is preferably operably linked to a promoter. Useful promoters include, for example, constitutive promoters, tissue specific promoters, and developmental stage specific promoters. Useful promoters also include inducible promoters such as, for instance, tet operator sequences (see, for instance, Bujard et al. (WO 96/01313)). A promoter used in one embodiment of the invention is a promoter provided by the Rosa26 locus. The ubiquitous Rosa promoter is provided by the ROSA26 mutant cell line, produced by the combination of embryonic stem cells with the ROSAβgeo retrovirus. Use of the Rosa26 locus is described in greater detail in Example 2, herein. The coding region encoding a transposase may also be operably linked to an enhancer. Enhancers are further defined herein. Enhancers are readily available, and are well known to those skilled in the art. See, for example, Blackwood et al., Science, 281, 60 (1998). Preferably, an enhancer is used in combination with a promoter. For example one embodiment of the invention uses the CAGGS combined promoter/enhancer, which is a chimeric promoter containing a chicken β-actin promoter and a cytomegalovirus enhancer. Use of the CAGGS promoter/enhancer is described in greater detail in Example 1, herein. The transposase coding region can be operably linked to the promoters and/or enhancers through use of a poly(A) trap vector, or by other means known to those skilled in the art.

Tissue-Specific Transposases and Tumor Models

An additional embodiment of the invention provides a transposase that is expressed predominantly in specific tissues. In once aspect, this accomplished through use of a tissue specific promoter that is operably linked to the transposase. A tissue-specific promoter is a promoter that is predominantly active in a particular tissue or tissues. Tissue-specific promoters are readily available, and are known to those skilled in the art. An example of a tissue-specific promoter is the probasin-promoter, which is a promoter that is specific for prostate epithelium prostate-specific antigen, and hence active primarily in the prostate. When used in combination with a transposon including an insertional mutagen, tissue-specific expression of a transposase provides a system in which insertional mutations can be induced in specific tissues. Expression in specific tissues can be useful, for example, to study cancer development in particular tissues, such as prostate or breast cancer, for example.

An additional embodiment of the invention provides a tissue-specific transposase by interrupting a promoter that is operably linked to the transposase with an interrupting nucleic acid sequence that renders the promoter inoperable. The interrupting nucleic acid sequence is typically flanked on each side by DNA recombinase cleavage sites. The interrupting nucleotide sequence is any nucleotide sequence that prevents the promoter from effectively functioning as a promoter. Such a promoter will be silent except in the presence of an appropriate DNA recombinase that excises the interrupting nucleotide sequence. By providing DNA recombinase in a tissue-specific fashion, such a promoter will act tissue-specifically. Exemplary DNA recombinase systems that can be used in this manner include the Cre-loxP system, and the yeast-derived Flp/frt recombinase system. Use of the Cre-loxP system will be described herein, but those skilled in the art appreciate that any DNA recombinase system can be used to provide a tissue-specific transposase. In addition, other methods are available for providing tissue-specific expression of transposases. For example, a tissue-specific transposase can be provided by coupling a DNA recombinase with a knock-in approach.

Cre recombinase is a site-specific DNA recombinase derived from P1 bacteriophage that recognizes the 34 base pair sequence SEQ ID NO:11 5′ ATAACTTCGTATAGCATACATTATACGAAGTTAT-3′, referred to as a loxP site. Cre may be provided in a tissue-specific fashion by generating a transgenic animal in which the Cre gene is expressed in a tissue-specific manner. The animal expressing the Cre gene is then intercrossed with an animal containing a transposase that has been silenced by including an interrupting nucleotide sequence flanked by loxP sites in its promoter. Animals generated from this breeding are transgenic for both constructs, and hence express Cre that will excise the interrupting sequence separated by the loxP sites, activating the promoter and moving it closer to the transposase. As Cre is expressed tissue-specifically, this results in the activation of the transposase gene predominantly in tissue in which the Cre gene is expressed.

To provide tissue-specific DNA recombinase expression, the DNA-recombinase encoding sequence may be operably linked to a tissue-specific promoter. As noted herein, tissue-specific promoters are readily available and known to those skilled in the art. For example, tissue-specific expression of Cre recombinase can be effected through use of various Cre alleles. For example, Probascin-Cre can be used to provide a transgenic animal that can be crossed with an animal encoding SB transposon to result in tumor formation in the prostate. Other examples include Villin-Cre, which provides GI tract and pancreatic tumors, Spc-Cre, which provides lung tumors, and Lysozyme-Cre, which provides myeloid leukemia. These can be used to make a transgenic animal which has both tissue specific DNA recombinase expression, and the presence of a transposase promoter that is activated by excision of an interrupting nucleotide sequence by the action of the DNA recombinase.

Transposition Frequency and Frequency Assay

In some aspects, an SB transposase of the present invention catalyzes the transposition of a transposon at a frequency that is greater than that catalyzed by a “baseline” transposase. Transposition frequency will typically increase based on the efficacy of the transposase and by providing increased levels of transposase. Preferably, the baseline transposase has the amino acid sequence of SEQ ID NO:5. Preferably, the transposon used to evaluate the ability of a transposase to mediate transposition has SEQ ID NO:6 as a left inverted repeat, SEQ ID NO:7 as a right inverted repeat, and a nucleic acid sequence of between about 1 kb to about 10 kb flanked by the inverted repeats. Preferably, the flanked sequence encodes a detectable marker and/or a selectable marker. Preferably, the coding region encodes resistance to an antibiotic, for example, the neomycin analog G418. For purposes of determining the frequency of transposition mediated by a transposase of the present invention, the activity of the baseline transposase is normalized to 100%, and the relative activity of the transposase of the present invention determined. Preferably, a transposase of the present invention causes transposition at a frequency that is, in increasing order of preference, at least about 50%, at least about 100%, at least about 200%, most preferably at least about 300% greater than a “baseline” transposase. Preferably, both transposons (i.e., the baseline transposon and the transposon being tested) are flanked by the same nucleotide sequence in the vector containing the transposons.

With an increase in transposition frequency, the likelihood of non-local transpositions increases due to secondary and further rounds of transposition. Local transpositions are those transpositions in which the transposon does not migrate a distance greater than 25 Mb from its initial location. High transposition frequency and additional rounds of transposition are preferred, as they result in a larger number of potential genes being affected by insertional mutations. An assay for measuring transposition using mammalian cell lines is provided in Example 3, herein.

The level of transposition can also be measured more directly through DNA analysis techniques. For example, the level of transposition in transgenic animals expressing both transposon and transposase can be determined by evaluating the level of transposon excision from somatic or germ line tissue. Excision normally leaves an excision product in the DNA that can be detected by analysis techniques including PCR and sequence analysis. For example, sequence analysis may be used to detect an excision repair product containing the CAG or CTG footprint that is known to occur in SB-mediated excision repair. Sequence analysis can thus be used to determine the level of excision occurring in tissue by, for example, determining how many cells contain excision repair products. In addition, if the size of a concatamer of transposons within an animal model is known, the level of excision can be determined by counting the diminished number of transposons within the concatamer over time, again using DNA analysis techniques including, for example, PCR and sequence analysis.

Transposase Analogs and Delivery Formulations

The SB transposases useful in some aspects of the invention include an active analog of SEQ ID NO:5, SEQ ID NO:20, or SEQ ID NO:21. An active analog can bind the inverted repeat sequences of SEQ ID NOs:6-7 and direct repeat sequences (SEQ ID NOs:13-17) from a transposon, as well as a consensus direct repeat sequence (SEQ ID NO:3 or SEQ ID NO:4). An active analog of an SB transposase is one that is able to mediate the excision of a transposon from a donor polypeptide.

Active analogs, as that term is used herein, include modified polypeptides. Modifications of polypeptides of the invention include chemical and/or enzymatic derivatizations at one or more constituent amino acids, including side chain modifications, backbone modifications, and—and C-terminal modifications including acetylation, hydroxylation, methylation, amidation, and the attachment of carbohydrate or lipid moieties, cofactors, and the like.

In addition to active analogs, active fragments of transposases may also be useful in some aspects of the invention. An active fragment of a transposase is a polypeptide that has been modified to have an incomplete amino acid sequence, generally by truncation at one end or the other, yet retains activity by being able to bind the inverted repeat sequences and mediate the excision of a transposase from a donor polypeptide and integration into a target site.

The present invention further includes polynucleotides encoding the amino acid sequence of SEQ ID NO:5, SEQ ID NO:20, or SEQ ID NO:21. An example of the class of nucleotide sequences encoding such the polypeptide disclosed in SEQ ID NO:5 is SEQ ID NO: 19, and the nucleotide sequences encoding the polypeptides disclosed at SEQ ID NO:20 and SEQ ID NO:21 can be easily determined by taking advantage of the degeneracy of the three letter codons used to specify a particular amino acid. The degeneracy of the genetic code is well known to the art and is therefore considered to be part of this disclosure.

The present invention further includes compositions that include a transposon of the present invention, a transposase of the present invention (either a polypeptide or a polynucleotide encoding the transposase), or both a transposon and a transposase. The compositions of the present invention optionally further include a pharmaceutically acceptable carrier. The compositions of the present invention may be formulated in pharmaceutical preparations in a variety of forms adapted to the chosen route of administration. Formulations include those suitable for parenteral administration (for instance intramuscular, intraperitoneal, in utero, or intravenous), oral, transdermal, nasal, or aerosol.

The formulations may be conveniently presented in unit dosage form and may be prepared by methods well known in the art of pharmacy. All methods of preparing a pharmaceutical composition include the step of bringing the active compound (e.g., a transposon, a transposase, or a combination thereof) into association with a carrier that constitutes one or more accessory ingredients. In general, the formulations are prepared by uniformly and intimately bringing the active compound into association with a liquid carrier, a finely divided solid carrier, or both, and then, if necessary, shaping the product into the desired formulations.

Methods for Introducing and Using Transposons and Transposases

The present invention also provides methods for introducing and using the transposons and transposases described by, for instance, Moran et al. (Cell, 1996; 87:917-927), Koga et al. (J. Hum. Genet., 2003; 48:231-235), and Miskey et al., (Nucl. Acids Res., 2003; 31:6873-6881), and the transposons and transposases described herein. For instance, the present invention includes a method for introducing a polynucleotide into the DNA of a cell, preferably, a vertebrate cell. The present invention also includes methods for providing cells including a transposase or polynucleotide sequences encoding a transposase. Preferably, the transposase is an SB transposase. A polynucleotide encoding a transposase may be integrated into the cell's genome or into extrachromosomal DNA. In an aspect of the invention, a vector can be used to insert a transposon or a polynucleotide encoding a transposase into a cell.

The method by which the transposon and/or transposase are introduced to the cell is not intended to be a limiting aspect of the present invention. For instance, the transposon and/or transposase can be introduced by anionic or cationic lipid, or other standard transfection mechanisms including liposomes, electroporation, particle bombardment, hydrodynamic injection, or microinjection used for eukaryotic cells. Preferably, the transposon and transposase are introduced to the cell by microinjection.

The cell may be ex vivo or in vivo. As used herein, the term “ex vivo” refers to a cell that has been removed, for instance, isolated, from the body of a subject. Ex vivo cells include, for instance, primary cells (e.g., cells that have recently been removed from a subject and are capable of limited growth or maintenance in tissue culture medium), and cultured cells (e.g., cells that are capable of extended growth or maintenance in tissue culture medium). As used herein, the term “in vivo” refers to a cell that is within the body of a subject.

The cell to which a transposon and transposase is delivered can vary. Preferably, the cell is a vertebrate cell. The vertebrate cell may be obtained from, for instance, a rodent such as a mouse or rat, livestock (e.g., pig, horse, cow, goat, sheep), a fish (e.g., zebrafish), or a primate (e.g., monkey). In some aspects, the cell is preferably a somatic cell.

The invention also provides a gene transfer system to introduce a polynucleotide into the DNA of a cell. The system includes a polynucleotide, or complement thereof, including a nucleic acid sequence flanked by first and second inverted repeats of the present invention, and an SB transposase of the present invention, or a nucleic acid encoding the SB transposase.

Methods of Making Transgenic Animals

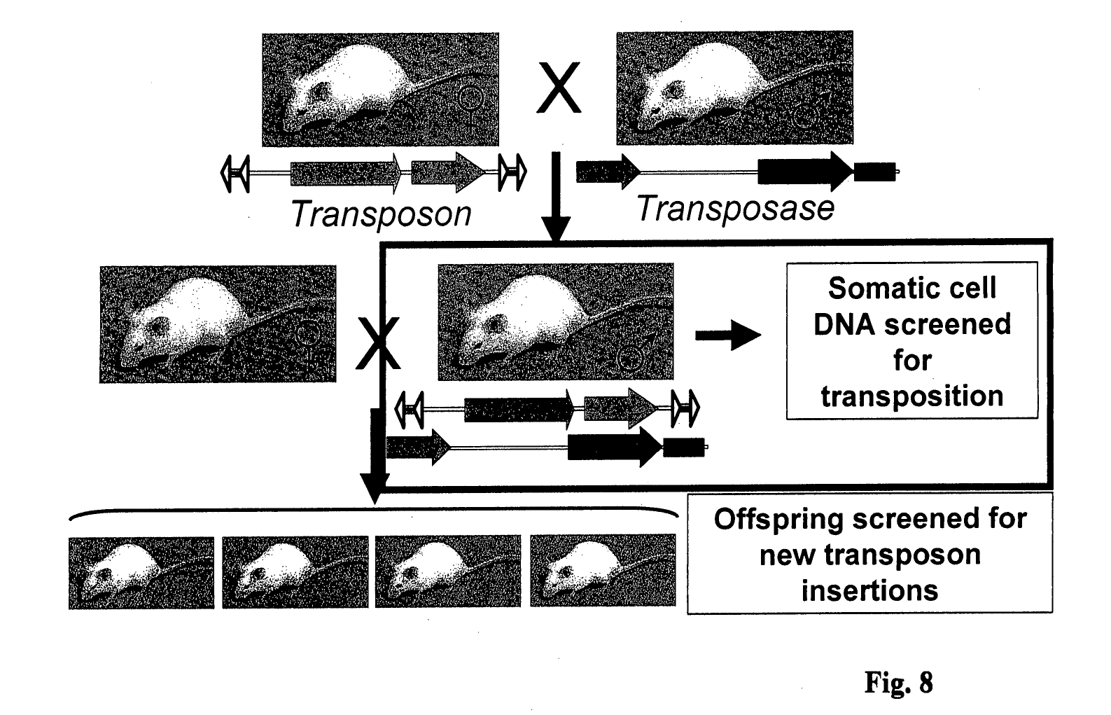

The present invention provides methods of making a transgenic animal that includes a transposon of the present invention in a germ cell. A transgenic animal, as defined herein, is an animal whose genome has been altered by the inclusion of a genetic element or genetic elements that are naturally present in another species. For example, the transfer of a transposase genetic element originally discovered in a salmonid fish to a mouse renders the mouse a transgenic animal. The present invention further provides methods of making transgenic animals that contain a polynucleotide encoding a transposase of the present invention in a germ cell. A further aspect of the invention includes making transgenic animals that contain both a transposon of the present invention and a polynucleotide encoding a transposase of the present invention in a germ cell. Transgenic animals containing both a transposon of the present invention and a polynucleotide encoding a transposase of the invention may be referred to herein as “doubly transgenic” animals. FIG. 8 shows the use of transgenic animals in which one animal containing a transposon in a germ cell is crossed with another animal containing a polynucleotide sequence encoding a transposase to provide a doubly transgenic animal that contains both the transposon and the transposase. This animal can, in turn, be crossed with another animal to generate further offspring, some of which may shown new transposon insertions.

As used herein, a “germ cell” is a male or female gamete cell (i.e., a spermatozoa or ovum), or one of their developmental predecessors. As a result of being in a germ cell, the transposon can be inherited by progeny of the animal. The transposon and/or polynucleotide encoding a transposase of the transgenic animals is integrated into the genomic DNA of a germ cell. As used herein, “genomic DNA” refers to the DNA present in a cell that is passed on to offspring. In some aspects of the invention, the animal is a rodent such as a mouse or rat, livestock (e.g., pig, horse, cow, goat, sheep), a fish (e.g., zebrafish), or a primate (e.g., monkey). Preferably the animal is a rodent, more preferably a mouse, and preferably the animal is not a human.

The cells to which the transposon and/or transposase are introduced are not intended to be a limiting aspect of the present invention. For instance, the cell can be a germ cell, a germ cell progenitor, a spermatogonial stem cell, a sperm cell, or an oocyte. Typically, if a haploid cell is used, the cell is fertilized after introduction of the transposon and transposase. Embryos, preferably one-cell embryos, can also be used. When the animal is a mouse, embryonic stem cells may also be used. Preferably, when the animal is a mouse, the cell is a one-cell embryo. Embryonic stem cells may also be obtained from a rat, bovine or porcine source.

The transposon introduced into the cell may be any of the transposons described herein. Transposons of the present invention include a polynucleotide that includes an insertional mutagen flanked by first and second inverted repeats. The insertional mutagen may include both disruptive sequences and affective sequences. The disruptive sequences may include splice acceptor sites operably linked to transcription termination signal sites, while the affective sequences may include promoters and splice donor sites. A preferred insertional mutagen is the provided by the T2/Onc2 polynucleotide, SEQ ID NO:19.

The polynucleotide sequence encoding a transposase introduced into the cell may encode any transposase that will excise a transposon of the invention. Preferably, the transposase encoded is a member of the SB family of transposases, or an active fragment thereof, and thus binds to the inverted repeats and mediates the excision and integration of a polypeptide flanked by inverted repeats. The polynucleotide sequence encoding the transposase may be introduced by any method known to those skilled in the art, and as described herein.

After a transposon and/or a polynucleotide encoding a transposase has been introduced to a cell, the resulting transgenic cell may be incubated under conditions that result in the formation of a transgenic animal that contains, in a germ cell, the transposon and/or the polynucleotide encoding a transposase. The incubation conditions that are appropriate vary depending on the animal and the type of cell used. When the animal is a mouse and the cell used is a one-celled embryo, the embryo is implanted into an appropriate female, and the cell is allowed to develop into a mouse. The resulting animal can be transgenic, in either a homogenous or mosaic fashion. The transposon and/or polynucleotide encoding a transposase present in such an animal is integrated into a germ cell of the transgenic animal, and the transgenic animal is thus capable of transmitting the transposon to its progeny. Preferably, the transposon is integrated into both germ cells and somatic cells of the transgenic animal. The invention is further directed to a transgenic animal that includes in a germ cell a transposon of the present invention that binds a transposase, a transgenic animal that includes in a germ cell a polynucleotide encoding a transposase, and a transgenic animal that includes a combination thereof. The invention is further directed to the progeny of any generation, preferably progeny of any generation that contain in a germ cell the transposon that binds a transposase. Preferably a germ cell that includes a polynucleotide encoding a transposase also includes an operably linked promoter as well.

The present invention also provides methods for mobilizing a transposon in a cell. Mobilization, as referred to herein, is defined as the excision of a transposon from a first site in the genomic DNA of the cell and subsequent reintegration to a second site in the genomic DNA of the cell. Subsequent mobilization from the second or later sites to reintegration at additional sites may occur as well. The excision and reintegration of mobilization are mediated by transposase. Preferably, the transposase is a member of the SB family of transposases. Thus, mobilization requires that both a transposon and a transposase that operates on that transposon be present in the same cell. This juxtaposition of transposon and transposase within a cell of a may be brought about using a variety of different methods. For instance, delivery of polynucleotides encoding both transposon and transposase to a cell may result in the presence of both the transposon and the transposase within the cell. Various methods known to the art can be used to determine if a transposon has been excised from a site or is present in a site. Without intending to be limiting, such methods include, for instance, inverse polymerase chain reaction (PCR), splinkerette PCR, and southern blot.

A cell used in the methods of mobilizing a transposon includes at least one transposon present in the genome. Preferably, the cell includes more than one transposon. For instance, the cell can include a transposon present on two or more chromosomes of the cell. Preferably, a chromosome contains a concatamer of transposons. A concatamer of transposons may be in any configuration, preferably, a head to tail configuration. Preferably, a concatamer of transposons includes at least two transposons, more preferably, at least about 25-50 transposons. In those embodiments where the cell includes more than one transposon, preferably each transposon is the same, i.e., the cell does not contain more than one type of transposon. While not intending to be bound by theory, concatamers are understood to form spontaneously, prior to integration into mouse chromosomal DNA, after injection of linearized plasmid DNA into a cell, for instance a one cell mouse embryo.

The present invention also provides methods for mobilizing a transposon in a cell that is part of a transgenic animal. Again, this involves the juxtaposition of a transposon and transposase within a cell of the transgenic animal. This may be accomplished by using a transgenic animal that contains the transposon in its germ and/or somatic cells, and then delivering a transposase. A transposase may be introduced to a cell as a polypeptide. When introduced in this fashion, it is preferred that the transposase polypeptide be fused to a second polypeptide that will more efficiently mediate transport of the transposase across the cell membrane. An example of a second polypeptide that can be fused to a transposase and mediate transport of a transposase across a cell membrane is the herpes simplex VP22 polypeptide (Wybranietz et al., J. Gene Med., 1999; 1:265-74). Alternatively, the transposase can be introduced to the cell as a polynucleotide encoding the transposase. When a polynucleotide encoding the transposase is introduced, the polynucleotide can be DNA or RNA, preferably RNA. A DNA polynucleotide including a coding sequence encoding a transposase can be introduced as, for instance, part of a plasmid vector or a viral vector. An mRNA encoding the transposase can also be introduced to a cell to provide transposase.

An additional method of juxtaposing transposon and transposase within a cell is cross breeding a transgenic animal having cells that contain the transposon with a transgenic animal having cells that are capable of expressing an appropriate transposase, resulting in the formation, among at least a portion of the offspring, of doubly transgenic animals that have cells that contain both transposon and transposase. The methods include providing a first animal that includes in a germ cell a transgenic coding sequence encoding a transposase, preferably a member of the SB family of transposases, and providing a second animal comprising, in a germ cell, a transgenic transposon, preferably a concatamer of transposons, present in the genome at a first site. The methods further include crossing the first animal with the second animal to obtain progeny. Progeny identified include a cell, including the transgenic transposon present in the genome of the cell in at least one second site. Methods of crossbreeding transgenic animals and categorizing the progeny are well known to those skilled in the art. An advantage of this method is that should doubly transgenic animals have a high level of morbidity due to a high level of mutation, new double transgenic animals can be readily generated by further crossbreeding of the original animals.

Alternatively, in another aspect, the methods include providing a first animal that includes in a germ cell a transgenic coding sequence encoding a member of the SB family of transposases and a transgenic transposon present in the genome at a first site. The methods further include crossing the first animal with a second animal to obtain progeny, where the second animal includes neither the transgenic coding sequence encoding a transposase present in the first animal nor the transgenic transposon present in the first animal. Progeny are identified that include a cell, preferably a germ cell, including the transgenic transposon present in the genome of the cell in at least one second site.

The invention is further directed to a transgenic animal made by these methods of mobilizing a transposon that bind a transposase, and the progeny of any generation, preferably progeny of any generation that contain in a germ cell the transposon that bind a transposase.

Use of Transposons as Insertional Mutagens

The present invention provides methods for using the transposons and transgenic animals described herein. The invention allows efficient insertion of genetic material into the genomic DNA of a cell of an animal for the mutation, evaluation of function, and subsequent cloning of genomic DNA, such as coding sequences and/or genomic regulatory sequences. In once aspect, the methods include providing a transgenic animal that includes in a germ cell a transposon that binds a transposase, preferably a SB transposase. The methods further include detecting an altered phenotype and/or the expression of a detectable marker. These methods include detecting an altered phenotype and/or expression of a detectable marker in an embryo obtained from the animal, in the adult animal, or in developmental stages between embryo and adult. In a preferred embodiment, the methods include detecting a tumor and subsequently mapping the location of the transposons present in a cell of the tumor. The locations of the transposons can then be used to identify the genomic coding sequences and or/genomic regulatory sequences altered by insertion of the transposons. By identifying genomic coding sequences and/or regulatory sequences whose alteration is commonly associated with a tumor or other phenotype, the function of these sequences may be characterized. For example, a genomic coding sequence and/or regulatory sequence that has been commonly altered in tumor tissue may be characterized as a tumor associated gene. Tumor-associated genes, as defined herein, include proto-oncogenes, oncogenes, and tumor suppressor genes.

Transposons of the present invention preferably include an insertional mutagen. The insertional mutagen increases the ability of the transposon to induce a tumor or other phenotypic change upon insertion into a genomic coding sequence and/or regulatory sequence of an animal. Alteration of the nucleic acid sequence will, in turn, affect the level of expression or the nature of the product expressed. When the nature of the product expressed is altered, the nucleic acid is referred to as a disruptive sequence. When the level of expression is altered, the nucleic acid is referred to as an affective sequence. A disruptive sequence can induce various types of mutations, including, for example, C-terminal truncations, N-terminal truncations, and insertion of promoters and/or enhancers.

A disruptive sequence may include a splice acceptor (SA) and a transcription termination signal site such as a polyadenylation (pA) signal site. Without intending to be limited by theory, if a transposon containing a disruptive sequence containing these sequences is inserted downstream from a gene, the SA and pA site combination will splice to the nearby gene and end transcription. Preferably, by providing a disruptive sequence with splice acceptor and transcription termination signals in both orientations, the transposon can act as a disruptive sequence when inserted in either orientation. Truncation of a protein, such as a kinase, for example, may result in the removal of regulatory regions of the kinase, converting the kinase from a proto-oncogene to an oncogene.

An affective sequence may include a splice donor (SD) and a promoter, such as an LTR promoter/enhancer. Without intending to be limited by theory, if an affective sequence is inserted upstream from a gene, the SD and LTR site combination will splice to a nearby gene and enhance the production of that gene via the promoter/enhancer activity of the LTR sequence. Should the gene affected be an oncogene or other gene that stimulates cell proliferation or other tumor-related activity, the resultant increased expression will also tend to encourage tumor formation.

Tumor formation is generally stimulated either by activation of an oncogene or proto-oncogene, or through inhibition of a tumor suppressor gene. Tumor formation may also be stimulated by genetic changes that result in a variety of subsequent cellular changes including, but not limited to, angiogenesis upregulation, growth factor independence, cell cycle progression, metastasis, invasiveness, inhibition of apoptosis, suppression of differentiation, and evasion of immune surveillance.

An oncogene, as defined herein, is a gene that can cause a cell to develop into a tumor cell. Oncogenes typically encode growth factors or protein kinases such as, for example, tyrosine kinases and GTPases. A proto-oncogene, as defined herein, is a gene whose protein product has the capacity to induce cellular transformation if it sustains a genetic insult. Activation to convert a proto-oncogene to an oncogene generally involves either a mutation of the proto-oncogene or an increased concentration of the product of the proto-oncogene, through an increase in product expression, stability, or gene duplication. A tumor suppressor gene, as defined herein, is a gene that reduces the probability that a cell will turn into a tumor cell. A mutation or deletion of such a gene will increase the probability of the cell containing the damaged gene to become a tumor cell. On the other hand, increased production of a tumor suppressor gene through, for example, increased promoter activity, can decrease tumor formation. Tumor suppressor genes also include growth suppressors, recessive oncogenes, and anti-oncogenes. Examples of proto-oncogenes, that can become oncogenes when mutated, include Erbb2, Kras, Src, Bcl2, and telomerase-encoding genes. Examples of oncogenes include erbB-2, Ras, Src, Bcl-2, and telomerase-encoding genes. Examples of genes that encode important tumor suppressors include p53, Rb, APC, and BRCA.

A transgenic animal of the present invention in which transposons have been mobilized (e.g., a doubly transgenic animal) may be assayed for the presence of a phenotype (e.g., a tumor) that is not present, or present to a different degree, in an animal at the same level of development that does not include the transposon integrated in its genome. Preferably, an altered phenotype can be identified visually by eye, for instance with the naked eye or with the aid of a dissecting microscope, by histological analysis, or by other methods appropriate to the phenotype being evaluated. In some aspects, for instance when the transposon includes a coding sequence encoding a detectable marker, mobilization of the transposon may also result in a detectable marker. Mobilization of transposons may also result in the formation of multiple phenotypes within an organism. For example, an insertionally oncogenic transposon may result in the formation of various different types of tumor tissue within an organism. Should various phenotypes (e.g. tumors) result, tissue regions exhibiting the differing phenotypes may be separated prior to genetic analysis.

Methods of Identifying Genes Altered by Transposons

Transgenic animals in which transposons have been mobilized may be further evaluated using various methods. For example, a transposon including a detectable marker may be used to identify a genomic coding or regulatory sequence using a technique known in the art as “gene trapping.” The polynucleotide includes a detectable marker that is not detectable unless it inserts into a genomic coding or regulatory sequence, or is operably linked to such a sequence. The method further includes detecting in the transgenic animal the detectable marker, wherein expression of the detectable marker indicates the transposon has integrated into a genomic coding sequence. Optionally, the animal can be assayed for the presence of a phenotype that is altered in comparison to an animal at the same level of development that does not include the transposon integrated in its genome.

In those aspects where the transposon includes a coding sequence encoding a detectable marker, the detectable marker may have distinct spatial and/or temporal expression. For instance, detection of the detectable marker only at specific times during the cell cycle or during development of the animal indicates that the transposon is inserted into the genomic DNA near or in a regulatory sequence or coding sequence that is active only at specific times (i.e., developmental stage-specific expression), or active only in specific tissues (i.e., tissue-specific expression).

Methods for mapping the location of a particular polynucleotide sequence such as a transposon are known in the art and are routinely used. Examples include, for instance, in situ hybridization, such as fluorescence in situ hybridization. In situ hybridization methods typically use a polynucleotide probe that is complementary to and will hybridize with nucleotides of the transposon. The conditions for hybridizing a polynucleotide probe to a transposon vary depending upon the polynucleotide sequence of the probe, and methods for determining such conditions are known in the art.

A preferred method for mapping the location of a particular polynucleotide sequence is determining the sequence of the cell's genomic DNA that flanks the transposon. Several methods are known in the art for determining the sequence of the cell's genomic DNA that flanks the transposon, and include, for instance, polymerase chain reaction (PCR) based methods. PCR based methods include, for instance, inverse PCR and various linker-mediated PCR techniques. Linkers are used in ligation-mediated (LM) PCR, for example, a cloning strategy that is used in a preferred embodiment of the invention to determine the location of transposon insertions. LM-PCR cloning is described in greater detail in Example 2, herein. Chromosomal flanking sequences can also be recovered using a plasmid rescue technique when the transposon includes sequences that support plasmid replication when introduced into E. coli.

Inverse PCR typically includes digesting the genomic DNA containing the transposon with a restriction endonuclease that does not cut the transposon, ligating the polynucleotides at a low concentration to promote intramolecular ligation, and then using primers that hybridize to different strands of the transposon and point outward from the transposon. The amplification takes place between the two primers and across the ligation junction, including both upstream and downstream chromosomal flanking sequences. The polynucleotide sequence of the amplified polynucleotide can then be determined, and compared to the known and publicly available databases containing genomic sequences of mammals such as, for instance, mouse, rat, or primate (e.g., monkey). Such methods are known to the art (see, for example, Hackett et al., WO 99/25817). The nucleotide sequence of primers useful for a PCR based method, and the conditions for amplifying a polynucleotide, will vary depending upon the nucleotide sequence of the transposon. Methods for determining useful primers and amplification conditions are routine in the art. A primer typically has at least 15 nucleotides, preferably, at least 20 nucleotides, most preferably, at least 25 nucleotides. A variety of primers used in embodiments of the invention are disclosed in Examples 1 and 2.

The location of the transposon can also be determined using a restriction endonuclease capable of cleaving a restriction site within the transposon. This yields at least one restriction fragment containing at least a portion of the integrated transposon, which portion includes at least a portion of an inverted repeat sequence along with an amount of genomic DNA of the cell that is adjacent to the inverted repeat sequence. The specificities of numerous endonucleases are well known and can be found in a variety of publications, e.g. Sambrook et al.; Molecular Cloning: A Laboratory Manual; Cold Spring Harbor Laboratory: New York (1989). The polynucleotide of the transposon thus preferably includes a restriction endonuclease recognition site, preferably a 6-base recognition sequence. Following insertion of the transposon into the genomic DNA of the cell, the DNA is isolated and digested with the restriction endonuclease. Where a restriction endonuclease is used that employs a 6-base recognition sequence, the cell DNA is cut into about 4000-base pair restriction fragments on average. Since the site of DNA insertion mediated by the transposase generally occurs at TA base pairs and the TA base pairs are typically duplicated such that an integrated nucleic acid fragment is flanked by TA base pairs, TA base pairs will be immediately adjacent to an integrated polynucleotide. The genomic DNA of the genomic fragment is typically immediately adjacent to the TA base pairs on either side of the integrated polynucleotide.

After the DNA of the cell is digested, the resulting fragments can be cloned in a vector using methods well known to the art, thereby allowing the identification of individual clones containing genomic fragments that include at least a portion of the inserted transposon and genomic DNA of the cell adjacent to the integrated transposon. A non-limiting example of identifying the desired genomic fragments is hybridization with a probe complementary to the sequence of the inverted repeats. Alternatively, linkers can be added to the ends of the digested fragments to provide complementary sequence for PCR primers. Where linkers are added, PCR reactions are used to amplify fragments using primers from the linkers and primers binding to a nucleotide sequence within the inverted repeats.

Correlation of Altered Genes with Tumor Formation

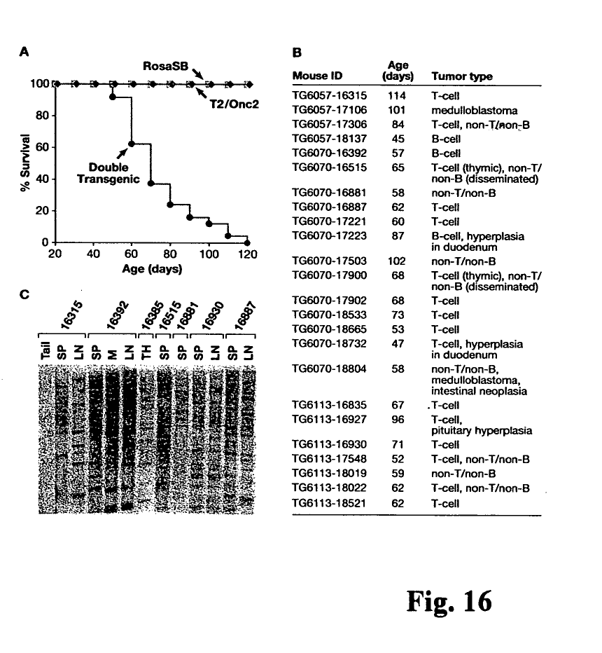

In further aspects of the present invention, methods may be used to determine the identity of tumor-associated genes. As described herein, use of transposons with flanked sequences that include insertional mutagens may lead to tumor formation as a result of a variety of genetic changes in the transgenic animals in which the transposons of the present invention have been mobilized by transposase of the present invention. For instance, FIG. 16A shows that at seven weeks of age, doubly transgenic mice in which transposons had been mobilized began to show signs of illness and by seventeen weeks, all the mice had died from cancer. Multiple tumor types were identified with the most common tumor being T-cell lymphoma, and single animals sometimes developed multiple cancer types (FIG. 16B). The degree to which transgenic animals in which transposons have been mobilized are prone to tumor development depends, in part, on the number of transposons present in cells of the animal and the level of expression of the transposase used. For example, in Example 1, use of the CAGGS-SB10 transposase did not result in tumor formation in wild-type mice, but did result in tumor formation in mice predisposed to cancer due to a deficiency in tumor suppressor p19. However, when the SB11 transposase was used in conjunction with the Rosa promoter, as in Example 2, transposase expression led to tumor formation in all animals, as shown in FIGS. 16A and 16B. Thus, the present invention can provide tumor formation in varying degrees.

Once tumor formation has been induced in an animal, the tumor tissue may be isolated and its genetic makeup characterized. The nature of the genetic changes associated with mobilization of transposons of the present invention within a cell or transgenic animal can be evaluated using the methods of genomic analysis described herein. This can be done to evaluate, for example, the genetic changes involved in formation of a particular tumor, identify groups of genes that are involved in tumor formation, evaluate tumor formation in particular tissues, and identify common genetic changes associated with a variety of tumors. For instance, the present invention can be used to identify tumor-associated genes in solid tumors.

In one aspect of the invention, the genetic changes associated with a particular tumor in a transgenic animal in which the transposons of the present invention have been mobilized may be characterized. An advantage associated with using transgenic animals is that the transposon and/or polynucleotides encoding transposase are already distributed in cells throughout the animal, facilitating the induction of tumors in tissue that might otherwise be relatively inaccessible. Animals used may be genetically modified organisms with particular traits. For example, animals that are predisposed to develop cancer may be used. Animals that are predisposed to develop cancer generally already bear oncogenes or altered tumor suppressor genes that facilitate the development of tumors in the animals. An example of a mouse line that is predisposed to develop cancer is the p19 Arf−/− mouse line. Other examples of animals predisposed to develop cancer include mice that lack the Trp53, Rb1, or Apc tumor suppressor genes. Furthermore, mice that conditionally express an oncogene, such as Kras(G12D) in just one tissue may be used. In a further aspect, mice that have been engineered so that a specific tumor suppressor gene, such as Pten, is homozygously deleted in just one tissue, such as the prostate, may also be used. Furthermore, animals may be evaluated for tumor formation during various stages of development. For example, in some aspects of the invention, it may be preferable to evaluate an embryo for tumor development, as tumor formation may occur earlier in the life cycle.

To identify tumor-associated genes in a transgenic animal in which transposons have been mobilized, one or more cells from a tumor identified in the animal are isolated. The DNA from the cell or cells is then evaluated to determine the location of transposon insertions within the genes of those cells, using the methods described or referred to herein. The locations of the transposons can then be used to determine the genes that are affected by the inserted transposons. If an insertion has occurred within a coding region, it is likely that the insertion has modified the protein expressed by that region. This may be confirmed by expression and analysis of the protein, if desired. Insertion into a protein involved in signal transduction or other proteins characterized as proto-oncogenic or tumor suppressive, are particularly likely to be discovered and associated with tumor development. If transposon insertion has occurred in a regulatory region, on the other hand, the importance of that regulatory region in stimulating or suppressing tumor formation may be identified. Insertion of transposons near coding or regulatory regions may also indicate involvement of nearby coding or regulatory regions in tumor formation or suppression.

A plurality of transposon insertions may be found in a tumor cell from a transgenic animal in which the transposons have been mobilized. For example, in tumors from doubly transgenic mice in which transposons were mobilized as described in Example 1, an average of 30 insertions per tumor were observed. In example 2, where a different transposase and promoter were used, an average of 50 insertions per tumor were observed. For a particular example of multiple insertions into an oncogene, see FIG. 9, which shows numerous different insertions at various different sites within the Braf gene. It is well known that cancer occurs through continual genetic evolution of mutant cells by a process of natural selection. Genetically abnormal cells are thought to be generated as a result of environmental insult or normal errors in replication. Some small fraction of these cells escapes normal controls on cell proliferation and increase their number. As this pool of mutant cells proliferates, additional mutant variants are continuously generated. If the result of these additional mutations provides a selective growth advantage, then the mutant variant will increase its relative number. During this process of cancer evolution, multiple cell-cycle checkpoints are generally dysregulated before tumor formation occurs. The identification of multiple transposon insertions and the coding and/or regulatory regions that can be provided by the present invention thus can help identify the systems or groups of genes that may be altered in carcinogenesis. See, for example, Table 1, below, in which transposons are shown to have integrated in the known oncogenes Braf, Ptprt, Ptch2, Rgs, Rabfap1, and Adarb2. Note also that while most tumors are genetically clonal due to the predominance of the cell capable of the highest level of survival and proliferation, a given tumor may also include cells with differing sites of transposon insertion, based on earlier or alternate pathways of cell growth dysregulation caused by transposon insertion.

The method of identifying tumor-associated genes of the present invention can also be used to evaluate the genetic basis behind the formation of a tumor in animal tissue. In one aspect, this may be accomplished through isolation of tumor cells from a particular tissue of a transgenic animal in which the transposons have been mobilized. Tumor formation may occur in a variety of tissues, which traditionally defines the nomenclature of the resulting cancer. Carcinoma is a cancer that begins in the skin or in tissues that line or cover internal organs. Sarcoma is a cancer that begins in bone, cartilage, fat, muscle, blood vessels, or other connective or supportive tissue. Leukemia is a cancer that starts in blood-forming tissue such as the bone marrow, and causes large numbers of abnormal blood cells to be produced and enter the bloodstream. Lymphoma is a cancer that begins in the cells of the immune system. The present invention can identify tumor-associated genes in any tissues where tumors may occur in a transgenic animal in which transposons have been mobilized. It is expected that nearly any type of tumor can be produced using the methods described herein. For instance, the present invention has been used to identify tumor-related genes in solid tumors. A solid tumor is defined herein as a tumor that typically contains few or no cysts or liquid areas. Carcinomas, sarcomas, and lymphomas often form a solid tumor, whereas leukemias generally do not.

In another aspect, the present invention provides a method of identifying tumor-associated genes that have been directed to occur only a specific tissue or tissues through use of tissue-specific transposase promoters, as described herein. An additional embodiment of the invention provides tissue-specific expression of transposase by interrupting a promoter that is operably linked to the transposase with an interrupting nucleic acid sequence flanked on each side by DNA recombinase cleavage sites that renders the promoter inoperable, and then providing DNA recombinase in a tissue-specific fashion, such that the transposase is only promoted in the desired tissues. Tissue-specific promoters can thus be used to investigate particular types of tumors, such as carcinomas, sarcomas, leukemias, and lymphomas, as well as individual tissue tumors included in these categories, such as medulloblastoma or intestinal carcinomas. An advantage of stimulating tumor formation in specific tissues is that it can potentially increase the lifespan of transgenic animals in which transposons of the present invention have been mobilized.

In a further aspect of the invention, genetic regions associated with tumor formation can be identified by determining the frequency of the insertion of a transposon in particular genetic regions in tumors isolated from a plurality of transgenic animals in which transposons have been mobilized. A genetic region associated with tumor formation has been identified as being associated with tumor formation with a “high probability” when such clustering of insertions with particular tumors has a probability of less than 5% by random chance. A genomic region mutated by the integration of a transposon in a plurality of different tumors is referred to herein as a common insertion site (CIS). The genomic region may be defined as a polynucleotide sequence with a particular length. For instance, evaluation of transposon insertions into sarcomas provided in Example 1 defined a CIS as 2 transposon integrations from 2 independent tumors within 13 kb of each other, or 3 or more transposon integrations from 3 independent tumors within 269 kb of one another. Alternately, a CIS may be defined as 2 or more transposon integrations from 2 independent tumors in the same gene. Note that a single integration, so long as it occurs within the same genomic region in more than one tumor, will qualify the genomic region as a CIS, and that the concept of a CIS is thus distinct from the concept of a plurality of insertions within a particular gene, discussed above. For example, Table 1 lists 54 different CIS that were identified by mobilizing the T2/Onc transposon in Arf −/− mice using the SB10 transposase. CIS identified by use of the present method thus provide genes or portions of genes that exhibit a significant likelihood of being a tumor-associated gene (e.g., a proto-oncogene, oncogene, or tumor suppressor gene).

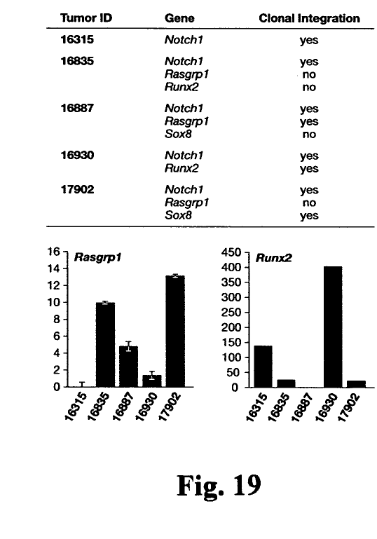

In a further aspect of the invention, cooperating genes associated with tumor formation can be identified in transgenic animals in which transposons have been mobilized. Cooperating genes are a plurality of genes that have an additive or synergistic effect in causing a cell to become a tumor cell when these genes have been altered by insertion of transposons of the present invention in or near the genes. For example, cooperating genes may be a plurality of genes that function to provide proteins involved in a particular signaling pathway. Cooperating genes include coding sequences that express proteins that interact, as well as regulatory sequences that may regulate the expression of coding regions. Identification of cooperating cancer genes and pathways can be helpful to understanding tumor development and preparing effective combinational therapies. For instance, in Example 2, among the six tumors with activating integrations at Notch1, three also had activating integrations upstream of Rasgrp1, a gene that positively regulates Ras signaling. Calculations demonstrate, as discussed in Example 2, that the probability of finding two tumors with integrations in the same two pairs of genes simply by chance is very low (p=9.2×10⁻⁵), suggesting that as integrations in Rasgrp1 were seen only in tumors with Notch1 integrations, Ras signaling appears to cooperate with Notch1 in tumor induction. As described in Example 2, data based on insertional mutations suggest that Sox8 and Runx2 may also represent genes that are cooperating with tumor formation associated with insertional mutation of the Notch1 and Rasgrp1 genes. The probability of finding integrations in three of the same genes in two independent tumors is exceedingly low (p=2.2×10⁻⁷), supporting the notion that these genes form a set of cooperating genes. FIG. 19 illustrates the cooperation of Notch1, Rasgrp,1, Sox8, and Runx2 genes implied by transposon integrations in those genes. Overall, Example 2 provides data suggesting that seven pathways were commonly disrupted by tumors induced by SB transposon insertion. Thus, the present invention is capable of providing data regarding a plurality of cooperating gene systems that are involved in tumor formation.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### Cancer Gene Discovery in Solid Tumors Using the CAGGS-SB10 Transposase and the T2/Onc Transposon in Tumor-Prone Mice

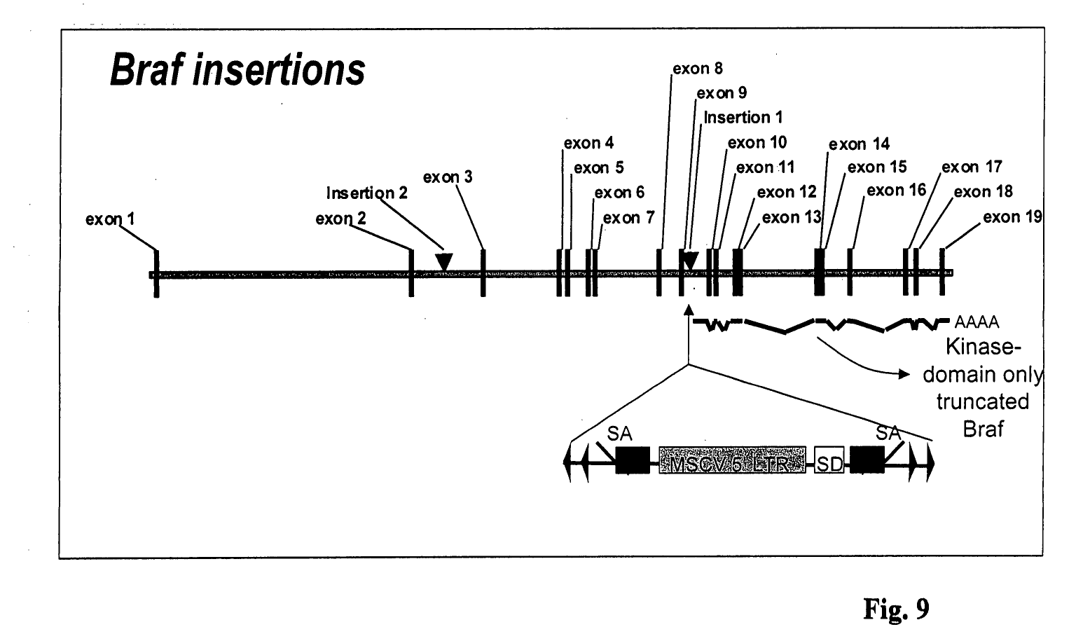

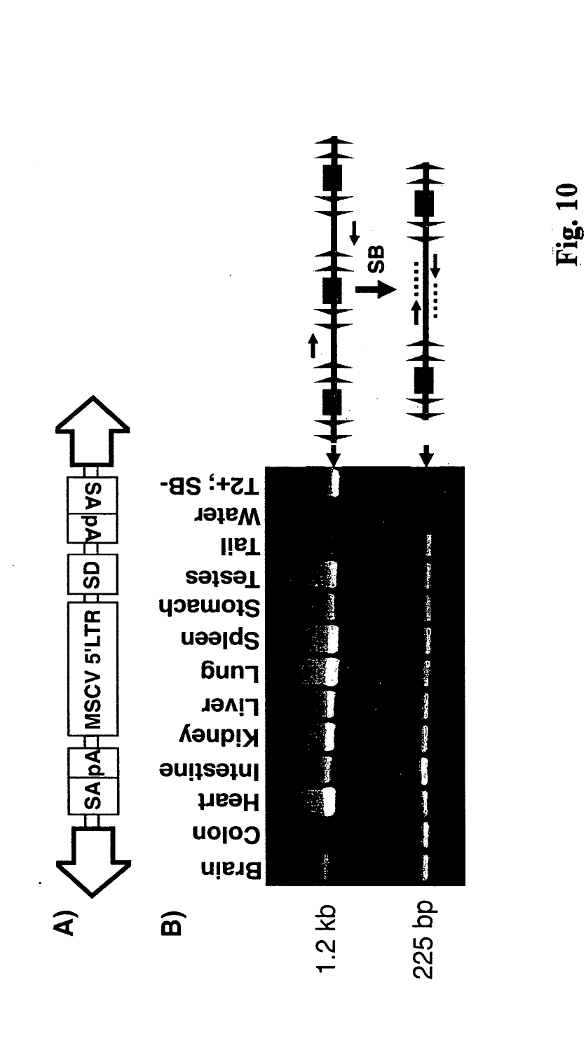

An SB transposon, called T2/Onc, was engineered to induce both loss- and gain-of-function mutations (FIG. 10A). T2/Onc contains splice acceptors followed by polyadenylation signals in both orientations to intercept upstream splice donors upon intronic insertion and generate loss-of function mutations. Between the two splice acceptors are sequences from the 5′LTR of the murine stem cell virus (MSCV), which contain strong promoter and enhancer elements that have been shown to be active in stem cells (Abdallah et al., Hum Gene Ther., 1996; 7:1947-54; Hawley et al., Gene Ther., 1994; 1:136-8; Cherry et al., Mol. Cell Biol., 2000; 20:7419-26). Immediately downstream of the LTR is a splice donor for splicing of a transcript initiated from the LTR into downstream exons of endogenous genes. Two lines (#68 and #76) of T2/Onc transgenic mice were used for analysis (see Example 1).

The ability of T2/Onc to mobilize in the soma was tested by breeding T2/Onc transgenic animals to transgenic mice expressing the SB transposase regulated by the ubiquitous CAGGS promoter (CAGGS-SB10) (Dupuy et al., Genesis, 2001; 30:82-8; Okabe et al., FEMS Lett., 1997; 407:313-9). CAGGS is a chimeric promoter derived from chicken β-actin and cytomegalovirus immediate early promoter sequences, and is ubiquitously active in transgenic mice. The pCAGGS-SB10 plasmid was constructed by cloning the 1,162 bp BamH1 fragment from pCMV-SB10 plasmid containing the SB10 open reading frame (SEQ ID NO: 8) into the Bgl II site of pCAGGS. Primers detect a 2.2 kb product (the size of the T2/Onc transposon) if a transposon has not mobilized from within the concatamer. If transposition and excision repair occurs anywhere within the concatamer, a 225 bp PCR product is generated. Excision of T2/Onc from the concatamer was detected in every somatic tissue tested from T2/Onc; CAGGS-SB10 doubly transgenic animals, but not in tissue from singly transgenic controls (FIG. 10B), while Southern blotting of normal tissue revealed few or no clonal, somatically-acquired T2/Onc insertions. New subclonal T2/Onc insertions (n=12) could be cloned from doubly transgenic somatic genomic DNA. These data showed that SB transposition occurs readily in somatic cells.

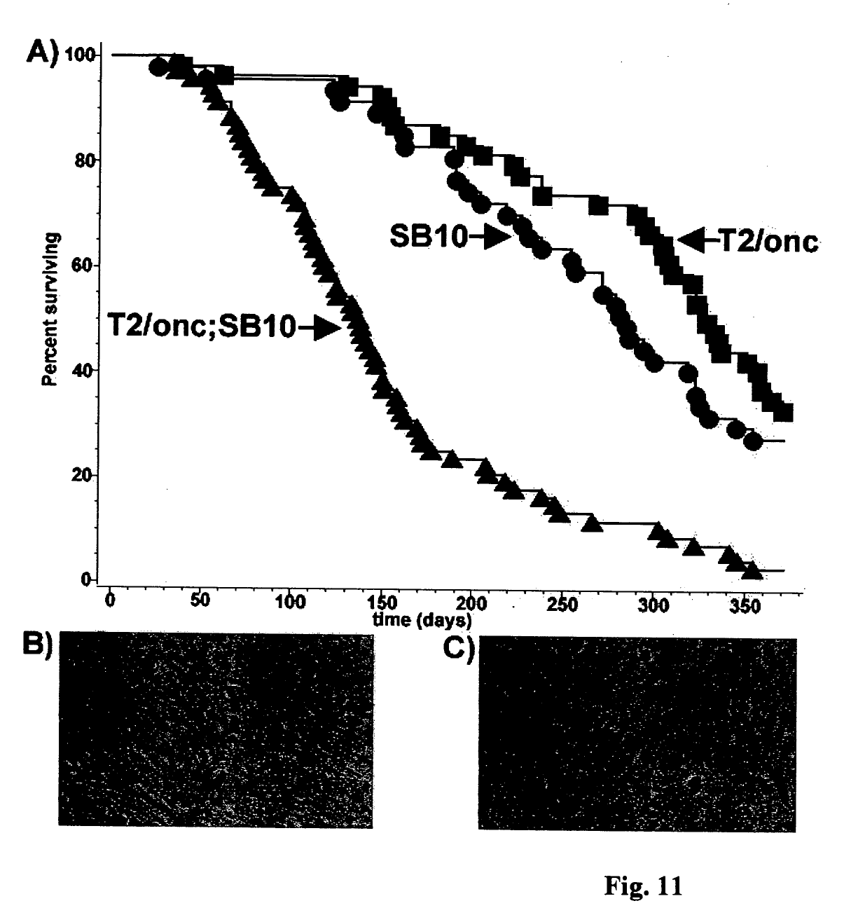

Mice doubly transgenic for both T2/Onc and CAGGS-SB10 were aged for greater than one year (n=26), but did not show evidence of cancer susceptibility different from background. It was hypothesized that somatic T2/Onc mobilization by CAGGS-SB10 alone is insufficient to promote rapid, highly penetrant tumor formation in wild-type animals, but may accelerate tumorigenesis in animals that are predisposed to cancer. Both T2/Onc concatamers and CAGGS-SB10 were crossed to Arf−/− mice, animals deficient for the p53 pathway regulator and tumor suppresser p19Arf13. Mice were generated on the Arf−/− background that carry T2/Onc, CAGGS-SB10, or both transgenes. The total number (n) and genotype of each group is indicated: Arf−/−; T2/Onc mice (n=54), Arf−/−; CAGGS-SB10 mice (n=48) and Arf−/−; T2/Onc; CAGGS-SB10 mice (n=64). A statistically significant decrease in time to morbidity in Arf−/− mice doubly transgenic for T2/Onc and CAGGS-SB10 compared to singly transgenic Arf−/−control animals (p<0.001, by Log Rank Mantel-Cox test) was observed (FIG. 11A). The tumor spectrum of Arf−/−; T2/Onc; CAGGS-SB10 mice was similar to that previously reported in Arf−/− mice on the C57BL/6 genetic background (Kamijo et al., Cancer Res., 1999; 59:2217-22). Thirty-six of fifty-two Arf−/−; T2/Onc; CAGGS-SB10 animals analysed had soft tissue sarcomas or osteosarcomas (FIG. 11B, 11C). Lymphomas, malignant meningiomas, myeloid leukaemias and a pulmonary adenocarcinoma were also observed. Comparing the two control groups, Arf−/−; CAGGS-SB10 and Arf−/−; T2/Onc, revealed no difference in time to morbidity (p=0.19, Logrank Mantel-Cox test).

Southern analysis of sarcoma genomic DNA from Arf−/−; T2/Onc; CAGGS-SB10 detected the presence of multiple, clonal, T2/Onc transposon insertions (average=5), while genomic DNA isolated from normal tissues from various locations of the same mice showed either zero or 1-2 subclonal T2/Onc insertions. Genomic sequences immediately flanking somatically-acquired transposon integration events in sarcomas from Arf−/− mice were amplified by linker-mediated PCR15. A total of 1053 distinct tumor-associated transposon integration events were cloned and sequenced from 28 tumors. In addition to cloning genomic integration events, the sequences immediately flanking the transgene concatamer in line #76 were obtained. One end of this concatamer donor locus was cloned and mapped to chromosome 1 at 164,879,699 bp. This was confirmed by fluorescence in situ hybridization (FISH).

In SB germline mutagenesis screens, 50-80% of transposons tend to reinsert within ˜6 megabases on either side of the donor concatamer (Vigdal et al., J. Mol. Biol., 2002; 323:441-52; Carlson et al., Genetics, 2003; 165:243-56; Horie et al., Mol. Cell Biol., 2003; 23:9189-207). For somatic transposition in sarcomas, this “local hopping” interval appears to be broadened as only 23% of somatic integrations cloned from tumors from #76 mice occurred within the 40 megabases surrounding the concatamer. The cloning of a large number of insertion sites also permitted mapping of the #68 concatamer donor to chromosome 15, confirmed by FISH, and revealed a similar percentage of local hopping.

A genomic region mutated by the integration of T2/Onc in multiple different tumors, a Common Integration Site (CIS), suggests selection for that event during tumorigenesis. Based on published Monte Carlo simulations, a CIS was defined as 2 integrations from 2 independent tumors in 13 kb, 3 or more integrations from 3 independent tumors in 269 kb, or 2 or more integration events from 2 independent tumors within the same annotated gene (Mikkers et al., Nat. Genet., 2002; 32:153-9; Johansson et al., Proc. Natl. Acad. Sci. USA, 2004; 101:11334-7). By these definitions, 54 CISs were identified by T2/Onc in Arf−/− sarcomas (Table 1).

TABLE 1
Common Integration sites in Arf-I-; T2/Onc; CAGGS-SB10 transgenic sarcomas.

| | mouse | approximate | | number of | number of |
|---|---|---|---|---|---|
| CIS name | chromosome | location | | integrations | independent tumors |
| Bai3 | 1 | 25.8 | Mb | 3 | 2 |
| Dst | 1 | 34.3 | Mb | 2 | 2 |
| ENSMUST00000042986.3 | 1 | 56 | Mb | 2 | 2 |
| Spag16 | 1 | 70 | Mb | 3 | 3 |
| ENSMUSG00000042581 | 1 | 129 | Mb | 2 | 2 |
| Daf1 | 1 | 130 | Mb | 3 | 2 |
| NG-1-143 | 1 | 143 | Mb | 3 | 3 |
| Uch15 | 1 | 143.7 | Mb | 4 | 4 |
| Rgs | 1 | 144 | Mb | 10 | 6 |
| B830045N13Rik | 1 | 146.8 | Mb | 6 | 6 |
| NG-1-147 | 1 | 147.2 | Mb | 4 | 4 |
| NG-1-147b | 1 | 147.7 | Mb | 3 | 3 |
| Laminin | 1 | 153 | Mb | 4 | 4 |
| Creg | 1 | 158 | Mb | 8 | 3 |
| C80879 | 1 | 159 | Mb | 4 | 3 |
| Rabgap1l | 1 | 160 | Mb | 17 | 8 |
| Tnfsf | 1 | 161 | Mb | 4 | 4 |
| Fmo | 1 | 162 | Mb | 3 | 3 |
| Prrx1 | 1 | 163 | Mb | 2 | 2 |
| Dpt | 1 | 164 | Mb | 4 | 4 |
| ENSMUSG00000038473 | 1 | 170 | Mb | 2 | 2 |
| Ptprt | 2 | 161 | Mb | 2 | 2 |
| Ptch2 | 4 | 115 | Mb | 2 | 2 |
| NG-6-23 | 6 | 22 | Mb | 2 | 2 |
| Cadps2 | 6 | 23.4 | Mb | 5 | 4 |
| Braf | 6 | 39 | Mb | 37 | 22 |
| E330009J07Rik | 6 | 40.3 | Mb | 4 | 4 |
| ENSMUST00000071875.1 | 6 | 43 | Mb | 3 | 3 |
| Cntnap2 | 6 | 46 | Mb | 3 | 3 |
| Baiap1 | 6 | 94 | Mb | 2 | 2 |
| ENSMUSESTT00000078632 | 12 | 83 | Mb | 3 | 3 |
| Adarb2 | 13 | 8.2 | Mb | 4 | 3 |
| ENSMUSG00000039828 | 15 | 7.8 | Mb | 2 | 2 |
| 4933421G18Rik | 15 | 8.1 | Mb | 2 | 2 |
| ENSMUST00000082227.1 | 15 | 16 | Mb | 4 | 3 |
| MGC92959 | 15 | 21.4 | Mb | 2 | 2 |
| 15-NG-22 | 15 | 22 | Mb | 3 | 3 |
| ENSMUSG00000043556 | 15 | 26 | Mb | 3 | 3 |
| ENSMUST00000075169.1 | 15 | 29 | Mb | 4 | 4 |
| Catnd2 | 15 | 30.5 | | 3 | 2 |
| Sema5a | 15 | 32 | Mb | 3 | 3 |
| Coh | 15 | 35.7 | Mb | 4 | 4 |
| Rims2 | 15 | 39.3 | Mb | 2 | 2 |
| 2610028F08Rik | 15 | 43.3 | Mb | 10 | 6 |
| Trhr | 15 | 43.9 | Mb | 25 | 11 |
| Csmd3 | 15 | 47.8 | Mb | 14 | 8 |
| ENSMUSESTG00000033246 | 15 | 48.8 | Mb | 3 | 3 |
| Rad21 | 15 | 52 | Mb | 6 | 4 |
| LOC277923 | 15 | 55 | Mb | 2 | 2 |
| BC026439 | 15 | 57.4 | Mb | 4 | 4 |
| NG-15-69 | 15 | 69 | Mb | 6 | 5 |
| ENSMUSESTG00000029680 | 15 | 70 | Mb | 4 | 3 |
| krt2 | 15 | 102 | Mb | 3 | 3 |
| ENSMUST00000074972 | X | 137.7 | Mb | 2 | 2 |

The “local hopping” phenomenon does increase the possibility of identifying CISs by random chance when they are linked to the concatamer donor locus. Based on published Monte Carlo simulations when insertions are distributed randomly and roughly 1000 independent insertions are studied, 10 of our 54 CISs are predicted to occur simply by random chance (Mikkers et al., Nat. Genet., 2002; 32:153-9). As SB transposon integration favors sites linked to the donor locus, traditional Monte Carlo simulation (that assumes a completely random distribution of insertions throughout the genome) cannot accurately predict the number of false CISs occurring at loci linked to the transposon donor locus. Therefore, our true false positive rate is likely higher than 10 as many CISs are linked to donor loci on chromosome 1 (for #76) and chromosome 15 (for #68) (Table 1). However, it is likely that some linked CISs are not merely identified by random chance. For example, several T2/Onc integrations in Arf−/− tumors occurred in Rabgap11, which is linked to the #76 donor locus on chromosome 1. Rabgap11 is a CIS identified in Example 2, below, providing additional evidence that Rabgap11 plays a role in tumorigenesis. Despite local hopping, it appears that the entire genome is accessible to SB somatic mutagenesis as T2/Onc integration events were cloned from all mouse chromosomes and CISs were also identified on chromosomes 2, 4, 6, 12, 13 and X. In addition, T2/Onc integrations were found near several CISs previously identified in leukemias or lymphomas in retroviral mutagenesis screens. Akagi et al., Nucleic Acids Res 32 Database issue, D523-7 (2004). Lund et al., Nat Genet 32, 160-5 (2002). See Table 2.

TABLE 2
Several T2/onc integrations occurred near previously
identified leukaemia retroviral CISs.

| Retroviral CIS | T2/onc integrations |
|---|---|
| Myc | 1 |
| Nfkb1 | 1 |
| Dst | 2 |
| Stk381 | 1 |
| Parvb | 1 |
| St13 | 1 |
| Rgs | 10 |

T2/onc integrations in tumors were compared against the RTCGD database (http://RTCGD.ncifcrf.gov) of CISs identified by retroviral mutagenesis screens in leukemia. Several single T2/onc integrations occurred near previously identified leukemia retroviral CISs. Dst and Rgs were CISs in both T2/onc and retroviral screens.

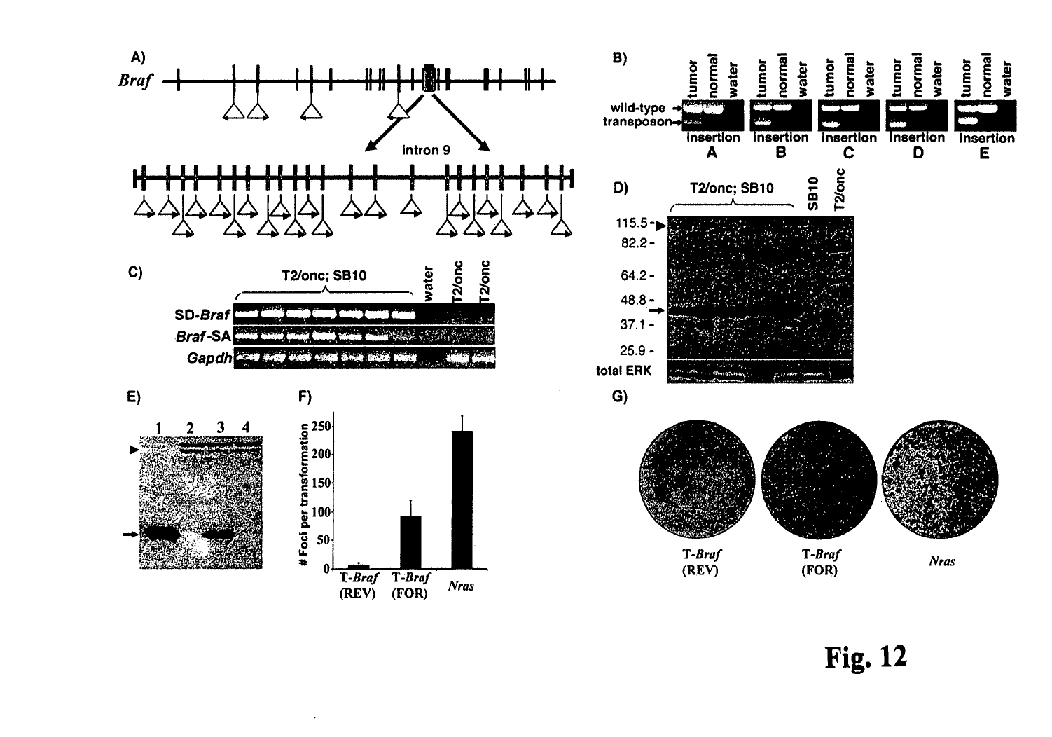

The gene most commonly disrupted by T2/Onc was Braf (Table 1). Integrations in or near Braf were cloned from 22 of 28 sarcomas and were found in tumors from mice transgenic for both T2/Onc concatamers, #68 and #76. Each sarcoma with Braf insertions had at least one within a TA dinucleotide in the ninth intron (FIG. 12A). All Braf ninth intron insertions analyzed appeared to be tumor specific and absent from normal tissue from the same mouse (FIG. 12B) and this is true of other T2/Onc gene insertions studied as well.

All ninth intron Braf integrations were directional with the MCSV LTR and splice donor oriented toward the tenth exon (FIG. 12A). This “sense” orientation predicts that transcripts initiated from the MCSV LTR would splice into the tenth Braf exon, and this was confirmed by RT-PCR in seven sarcomas (FIG. 12C). This transcript could result in the expression of a truncated protein, translationally initiated in exon 10, containing the kinase domain of BRAF. An antibody against the C-terminal fragment of BRAF detected a protein of the expected size (˜40 kDa) specifically in 5 sarcoma lysates that harbor intron 9 Braf gene T2/Onc insertions (FIG. 12D). Moreover, a N-terminal specific BRAF antisera did not detect a truncated BRAF peptide despite the fact that a truncated Braf mRNA, generated by splicing from the Braf exon 9 splice donor into the splice acceptor upstream of the MSCV LTR sequences in T2/Onc, was detected (FIG. 12C). Thus, the data demonstrates that the T2/Onc splice donor splices into the tenth exon of Braf (SD-Braf) and the Braf exon nine splice donor splices into the T2/Onc splice acceptor (Braf-SA).

Braf is a known oncogene, which has been shown to contain activating point mutations in 9% of human sarcoma cell lines and 0.5-5% of primary human sarcomas (Davies et al., Nature, 2002; 417:949-54; Seidel et al., Int. J. Cancer, 2005; 114:442-7). This provides proof of principle that SB somatic mutagenesis identifies genes associated with and clinically relevant to specific human cancers. The truncated BRAF protein expressed in sarcomas with T2/Onc integrations in the ninth intron of Braf contains only the kinase domain and lacks N-terminal negative regulatory elements of the protein and is capable of morphological transformation of NIH 3T3 cells (FIG. 12E-G). Previous work has demonstrated the oncogenic potential of a truncated kinase domain of the closely related Craf25. Based on these results, it appears that Braf is capable of collaborating with Arf loss to elicit sarcoma development.

The data presented shows that Sleeping Beauty can be utilized for somatic-cell insertional mutagenesis in the mouse for the identification of cancer genes in solid tumors. T2/Onc mobilization combined with tissue-specific loss of a tumor suppresser may allow for the identification of tumor-predisposing genes for any tissue type. In Example 2 it is shown that the improved SB transposase SB1126, expressed from the Rosa26 locus, can cause efficient somatic mobilization of a T2/Onc-like transposon and induce tumor formation in the absence of a cancer predisposed genetic background. The differences in the ability of T2/Onc mobilization by CAGGS-SB10 and Rosa26-SB11 to initiate and promote tumor formation may be due to differences in activity of SB10 and SB11, differences in levels of protein expression, or differences in spatial/temporal expression of the transposase. The use of a conditionally-expressed SB transposase may improve future studies by allowing control of its spatial and temporal expression. In addition, new transposon vector designs may further enhance the utility of the system.

Experimental Methods

Vector Construction

The T2/Onc vector contains the MSCV 5′ long terminal repeat from the MSCVneo vector (Clontech). The splice donor is from exon 1 of the mouse Foxf2 gene. One splice acceptor is derived from exon 2 of the mouse engrailed-2 gene and the other from the carp β-actin gene. Each are followed by the bidirectional SV40 poly(A).

Mice

Transgenic lines of T2/Onc were generated on the FVB/N genetic background. Southern analysis was performed on tail biopsy genomic DNA, and two lines (#68 and #76) with high copy numbers (approximately 25 copies) and a lack of transgene methylation were chosen for further analysis. The copy number of T2/Onc elements within the concatamer was estimated by comparison of T2/Onc signal intensity from transgenic genomic DNA to known amounts of T2/Onc plasmid DNA by Southern analysis. Methylation status was investigated by Southern analysis after digestion with a methylation sensitive restriction enzyme. It was hypothesised that methylation of the transposon transgene may silence the activity of the MSCV LTR after transposition to new sites in the genome. FISH and SKY analysis were used to map concatamer #68 to chromosome 15 and concatamer #76 to chromosome 1. To generate the Arf−/− cohort, Arf+/−; CAGGS-SB10 and Arf+/−; T2/Onc mice were first generated by crossing Arf−/− mice to CAGGS-SB10 or T2/Onc mice, respectively. Arf+/−; CAGGS-SB10 mice were intercrossed to generate Arf−/−; CAGGS-SB10 mice. Arf+/−; T2/Onc mice were also intercrossed to generate Arf−/−; T2/Onc mice. Arf−/−; CAGGS-SB10 mice and Arf−/−; T2/Onc mice were crossed to generate Arf−/−; CAGGS-SB10; T2/Onc, Arf−/−; T2/Onc and Arf−/−; CAGGS-SB10 mice.

T2/onc Excision PCR

50 ng of genomic DNA was used for PCR. Primers used for excision PCR were as follows: 5′-TGTGCTGCAAGGCGATTA-3′ (SEQ ID NO:35) and 5′-ACCATGATTACGCCAAGC-3′ (SEQ ID NO:36).

Histopathology

Tissues were fixed in 10% formalin overnight at 4° C., stored in 70% ethanol, paraffin embedded, sectioned and stained with hematoxylin and eosin.

Linker-Mediated PCR and “Shot-Gun Cloning”

Linkers used to clone insertions from the IR/DR(R) were generated by annealing primers 5′-GTAATACGACTCACTATAGGGCTCCGCTTAAGGGACCATG-3′ (SEQ ID NO:37) and 5′-Phos-GTCCCTTAAGCGGTAAAG-NH₂-3′ (SEQ ID NO:38). Linkers used to clone insertions from the IR/DR(L) were generated by annealing primers 5′-GTAATACGACTCACTATAGGGCTCCGCTTAAGGGAC-3′ (SEQ ID NO:39) and 5′-Phos-TAGTCCCTTAAGCGGAG-NH₂-3′ (SEQ ID NO:40). Genomic DNA was digested with NlaIII and XhoI (for cloning from IR/DR(R)) or BfaI and BamHI (for cloning from IR/DR(L)) and ligated to the linker. Primary PCR primers used to amplify sequences flanking the IR/DR(R) were 5′-GTAATACGACTCACTATAGGGC-3′ (SEQ ID NO:41) and 5′-GCTTGTGGAAGGCTACTCGAAATGTTTGACCC-3′ (SEQ ID NO:42). Primary PCR primers used to amplify sequences flanking the IR/DR(L) were 5′-GTAATACGACTCACTATAGGGC-3′ (SEQ ID NO:43) and 5′-CTGGAATTTTCCAAGCTGTTTAAAGGCACAGTCAAC-3′ (SEQ ID NO:44). Primary PCR was diluted 1:50 and used in a secondary PCR. Secondary PCR primers used to amplify sequences flanking the IR/DR(R) were 5′-AGGGCTCCGCTAAGGGAC-3′ (SEQ ID NO:45) and 5′-CCACTGGGAATGTGATGAAAGAAATAAAAGC-3′ (SEQ ID NO:46). Secondary PCR primers used to amplify sequences flanking the IR/DR(L) were 5′-AGGGCTCCGCTAAGGGAC-3′ (SEQ ID NO:47) and 5′-GACTTGTGTCATGCACAAAGTAGATGTCC-3′ (SEQ ID NO:48). Secondary PCR products were ligated to pGEM®-T Easy (Promega) and electroporated into DH10B Electromax competent cells (Invitrogen). Library plating, colony picking and sequencing using the SP6 primer in 96-well format was performed by Agencourt Biosciences. Automated database searches of T2/Onc integration sites were performed as previously described by Akagi et al., Nucleic Acids Res. 32, D523-7 (2004). The closest gene to each integration (within 100 kb on either side of the integration) was determined using a combination of the UCSC (http://www.genome.ucsc.edu and Ensembl (http://www.ensembl.org) mouse whole genome annotations (NCBI m33 build).

Braf Three-Primer PCR

500 ng genomic DNA was used in a PCR with three separate primers. One transposon specific primer was used for all three-primer PCR: 5′-GTGGTGATCCTAACTGACCT-3′ (SEQ ID NO:49). Primers used to detect the wild-type locus of each cloned insertion event are: Braf Insertion A: 5′-CGTAGTTATCATTTATTGGTAGCAG-3′ (SEQ ID NO:50) and 5′-GGAAAGCTAGATGGAAATTC-3′ (SEQ ID NO:51), Braf Insertion B: 5′-CCATGCCTGTGCATTTGTTATG-3′ (SEQ ID NO:52) and 5′-GCACAGATGCTTACCATCCG-3′ (SEQ ID NO:53), Braf Insertion C: 5′-GCAAACTCTGTAATAATGTACC-3′ (SEQ ID NO:54) and 5′-CTAAGCAGGCTGTTTACTAC-3′ (SEQ ID NO:55), Braf Insertion D: 5′-CTGTCCCCAGTGAAATAGTG-3′ (SEQ ID NO:56) and 5′-CTCAAGTGCTGAAGTTTCAG-3′ (SEQ ID NO:57), Braf Insertion E: 5′-ATAATCCAGTGATAAGAACTGTGC-3′ (SEQ ID NO:58) and 5′-CAGCCAGTGCTTATAAACTG-3′ (SEQ ID NO:59).

Braf RT-PCR

Total RNA was isolated from tumour tissues using TRIzol® (Invitrogen). Contaminating DNA was removed by DNase treatment (Invitrogen). RT-PCR was performed using 500 ng of RNA with the RobusT I RT-PCR Kit (Finnzymes, MJ Research). A SD specific primer 5′-GAACGCCCGCGAGGATCTCT-3′ (SEQ ID NO:60) and a Braf tenth exon specific primer 5′-CTTCTGTCCTCCGAGGATGA-3′ (SEQ ID NO:61) were used. A Braf exon seven specific primer 5′-GAGCATCACCCAGTACCACA-3′ (SEQ ID NO:62) and a Carp β-actin SA specific primer 5′-ACGTTGCTAACAACCAGTGC-3′ (SEQ ID NO:63) were used. The resulting products were sequenced to verify fidelity of each splicing event.

BRAF Western Analysis

Protein lysates were prepared by homogenising tissue in IPWB lysis buffer [50 mM Tris (pH7.4), 14.6 mg/mL NaCl, 2 mM EDTA, 2.1 mg/mL NaF, 1% NP-40, 1 mM NaVO₄, 1 mM Na₂PO₄, with protease inhibitors (Roche)] or by following manufacturer's protocols for protein isolation from the organic phase of TRIzol® (Invitrogen). Samples (˜30 μg) were electrophoresed on a 4-12% Bis-Tris gel and transferred to nitrocellulose (BIO RAD) using the NuPAGE system (Invitrogen). Blots were probed with a primary antibody specific to the carboxy terminus of BRAF (Santa Cruz Biotechnology). An antibody specific for Erk-1 (Santa Cruz Biotechnology) was used as a loading control.

Cloning of Truncated Braf and NIH3T3 Transformation Assay

The cDNA of the truncated C-terminal fusion transcript of Braf generated in tumors (T2/Onc SD-exons 10-19 of Braf) was amplified using the RobusT I RT-PCR Kit (Finnzymes, MJ Research) with the following primers: 5′-CAGTCCTCCGATAGACTGCG-3′ (SEQ ID NO:64) and 5′-GGACTGGCTACTTGAAGGCT-3′ (SEQ ID NO:65). The amplified product was subcloned into pCR®2.1-TOPO® (Invitrogen), excised with EcoRI and subsequently ligated in the forward and reverse orientations into an EcoRI site of a CAGGS vector. These plasmids as well as a CAGGS plasmid expressing the activated human NRAS oncogene (G 12V) were each transfected in duplicate into NIH3T3 cells using the SuperFect Transfection Reagent (Qiagen). Cells were cultured in DMEM with 10% FBS, 2 mM L-Glutamine, 0.1 mM non-essential amino acids, 55 μM P-Mercaptoethanol, and 10 μg/ml Gentamycin, split two days post-transfection into two 100 mm plates, cultured for 10 days and stained with methylene blue.

### Example 2

### Mammalian Mutagenesis Using the Rosa26-SB11 Transposase and the pT2/Onc2 Transposon

Creating a Highly Active SB Mutagenesis System

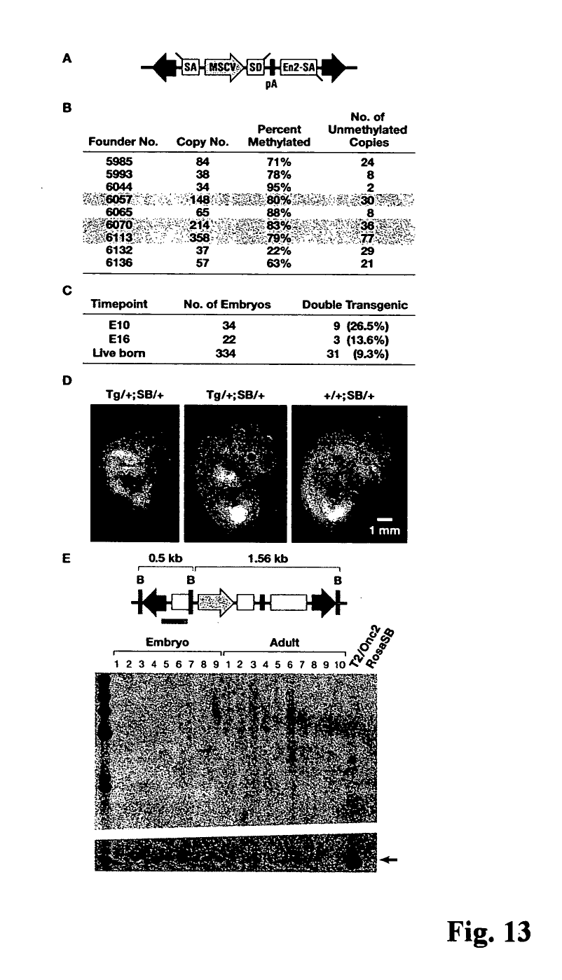

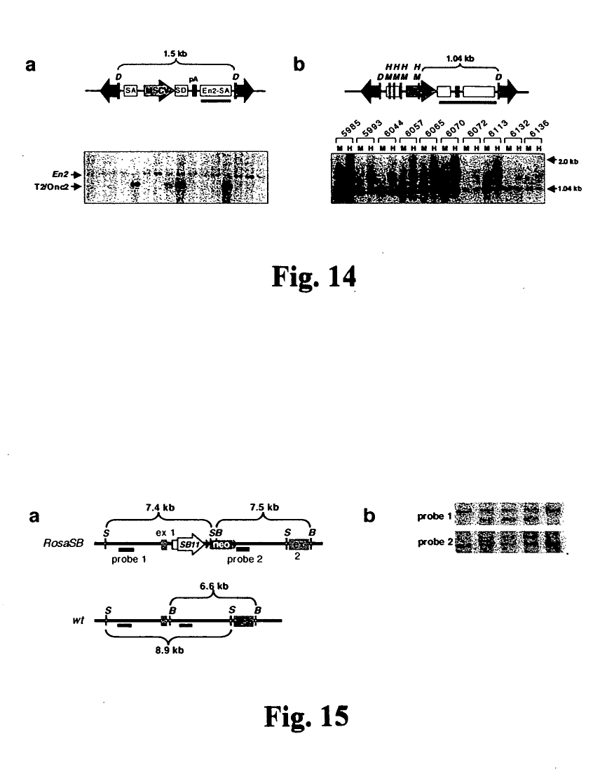

To develop a more active eukaryotic SB transposition system, a number of enhancements were made to the SB transposition system used previously. For example, a mutagenic transposon vector, T2/Onc2 was generated (FIG. 13A). This transposon is similar to that described by in Example 1, but contains a larger fragment of the engrailed-2 (En2) splice acceptor (SA) and is flanked by optimized SB transposase binding sites that increase SB transposition (Cui et al., J. Mol. Biol., 2002; 318:1221-1235). It is also smaller than other SB transposons used previously (˜2.0 kb) and approaches optimal size for transposition (Geurts et al., Mol. Ther., 2003; 8:108-117). T2/Onc2 contains two splice acceptors and a bi-directional polyA (pA) and can terminate transcription when integrated in either orientation in a gene. It also contains a murine stem cell virus (MSCV) long terminal repeat (LTR) and a splice donor (SD) and can promote gene expression when integrated upstream or within a gene. Thirty T2/Onc2 transgenic founders were generated following microinjection. Since SB transposes by a cut-and-paste mechanism, the number of transposons in the transgene concatamer can initially limit the number of transposition events. Any methylation present on the transposon could also be transferred to new sites within the genome. Methylation of the MSCV promoter might therefore inhibit its ability to affect expression of neighboring genes. With this in mind, founder transgenic animals were screened to determine their transposon copy number and methylation status of the MSCV promoter (FIG. 13B, FIG. 14). Three founder transgenic animals containing a high copy number of unmethylated transposons were used to establish transgenic lines (FIG. 13B). Transposon concatamers from each line were transmitted at normal Mendelian frequencies and heterozygous mice showed no obvious phenotype.

Next, a transposase knock-in allele was generated to avoid epigenetic silencing often seen with transgenes. To increase SB transposition, the knock-in was generated using the SB11 transposase (Geurts et al., Mol. Ther., 2003; 8:108-117). This transposase contains four amino acid substitutions that increase its activity above that of the SB10 transposase used previously. An expression cassette consisting of a splice acceptor site upstream of the SB11 cDNA followed by an SV40 polyadenylation signal was targeted to the Rosa26 locus to generate the RosaSB allele (FIG. 15). This site was chosen because genes targeted to this locus are ubiquitously expressed during development and in adult mouse tissues. Western blotting confirmed expression of the SB11 transposase in RosaSB mice, and quantitative PCR indicated the RosaSB allele is equally expressed in all tissues tested (brain, spleen, skin and lung). Heterozygous RosaSB mice were aged for over a year and showed no obvious phenotype.

RosaSB mice were then crossed to each T2/Onc2 transgenic line to generate a cohort of mice harboring both elements. Unexpectedly, intercross offspring showed a non-Mendelian inheritance pattern with a significant decrease in progeny inheriting both the RosaSB transposase and the T2/Onc2 transgene. All three T2/Onc2 lines produced fewer double transgenic progeny than expected, although the frequency varied among the lines [TG6070, 17/136 (12.5%); TG6057, 5/89 (5.6%); TG6113, 9/109 (8.3%)]. It is hypothesized that this decrease in viability was due to lethality induced by SB transposition and/or DNA damage that was not repaired following SB excision. Previous studies using mice deficient for proteins involved in nonhomologous end joining indicate that lymphocytes and neurons are particularly sensitive to double strand breaks during development (Gao et al., Cell, 1998; 95:891-902; Barnes et al., Curr. Biol., 1998; 8:1395-1398). To test this hypothesis, embryos were characterized at various developmental time points. Normal frequencies of double transgenic embryos were observed at E10 while a significant decrease was seen by E16 (FIG. 13C). Embryos at both time points appeared grossly normal, although many double transgenic embryos appeared smaller than control littermates (FIG. 13D). In FIG. 13D, Tg/+ is the genotype of the transposon transgene, while SB/+ is the genotype of the transposase transgene. Histopathological examination of double transgenic embryos showed various developmental abnormalities unique to each embryo.

To determine whether SB transposition occurs in double transgenic embryos, the question of whether SB transposons have been excised from transposon concatamers in double transgenic embryos was evaluated, since excision is the first step in transposition. BamHI sites are located within the plasmid sequences that flank each transposon in the concatamer and in the transposon itself (FIG. 13E). Consequently, any transposon in the concatamer will generate a 500 bp fragment using the probe indicated. It is unlikely that BamHI sites will immediately flank a transposon following transposition. Reintegrated transposons will therefore primarily generate BamHI fragments that are larger than 500 bp. Analysis of nine double transgenic embryos showed that most transposons were excised from the concatamer by E10 (FIG. 13E). Analysis of brain and kidney of ten adult double transgenic animals showed that transposon excision continues in the adult until by postnatal day 45 virtually all of the transposons within the concatamer are excised (FIG. 13E). Excision thus begins early in development and continues into the adult, affecting virtually all cell types.

Previous studies showed that 75% of excised transposons reintegrate into the mouse genome (Luo et al., Proc. Natl. Acad. Sci. USA, 1998; 95:10769-10773). To confirm that excised transposons reintegrate into the mouse genome, ligation-mediated PCR (LM-PCR) was used, using the procedure of Wu et al. (Science, 2003; 300:1749-1751), to amplify SB junctions from 10 double transgenic embryos. LM-PCR is a powerful new amplification method that makes it possible to rapidly amplify and sequence thousands of SB transposition sites. Ninety-six SB junction fragments were randomly picked and sequenced from each amplified embryo library. BLAST searches of the 490 independent transposon junctions showed that most junctions were rare and represented only once among the clones analyzed, indicating that each junction is present in a limited number of cells. This is consistent with Southern data, which showed no detectable newly acquired SB transposons in double transgenic embryos (FIG. 13E). SB transposons are therefore reintegrating into the mouse genome at many sites in double transgenic mice.

T2/Onc2 concatamer integration sites are located on chromosomes 1, 4 and 6. As expected, an increased frequency of transposons reintegrated on these chromosomes was observed in double transgenic embryos. The percent local transposition within a 25 Mb region varied from 6-11% in double transgenic embryos in contrast to germline transpositions reported by others where 50-80% of the transpositions were local (Horie et al., Proc. Natl. Acad. Sci. USA, 2001; 98:9191-9196; Fischer et al., Proc. Natl. Acad. Sci. USA, 2001; 98:6759-6764; Carlson, et al., Genetics, 2003; 165:243-256). Even when transposons landed on the same chromosome as the transgene concatamer, the transposon integrations were well distributed across the chromosome and there was no easily defined local hopping interval. The higher SB transposition frequencies obtained with this system may allow for secondary and tertiary rounds of transposition, which masks local transposition. This high rate could be attributed to a more optimal expression of the SB11 transposase from the RosaSB allele. Previous work has indicated that even moderate changes in SB transposase expression can have a significant impact on transposition frequency (Geurts et al., Mol. Ther., 2003; 8:108-117).

SB transpositions in the embryo are fairly well distributed across the genome. When T2/Onc2 integrated in or near a gene there was little preference for a gene region or orientation relative to the nearest gene. See Table 3. Only four regions in the genome (<30 kb in size) contained two SB transposon integrations in independent embryos (Table 4), which is similar to the number (3) predicted by Monte Carlo simulations for random integration, and no region (<100 kb in size) contained 3 SB transposon integrations. The embryo data therefore appears to represent a population of unselected transposon integrations.

TABLE 3
Comparison of transposon integration
sites cloned from embryos and tumors
Overview of integration site distribution

| | |
|---|---|
| Embryo | Tumor |

| | | | | |
|---|---|---|---|---|
| In genes | 118 | (24%) | 239 | (30%) |
| 5′ of genes | 88 | (18%) | 153 | (20%) |
| 3′ of genes | 84 | (17%) | 117 | (15%) |
| >100 kb from gene | 201 | (41%) | 273 | (35%) |
| | 491 | | 782 | |

Orientation of transposon integrations in
embryos relative to nearest gene

| | | |
|---|---|---|
| In genes | 5′ of gene | 3′ of gene |

| | | | | | | |
|---|---|---|---|---|---|---|
| same | 64 | (54%) | 46 | (52%) | 48 | (57%) |
| inverse | 54 | (46%) | 42 | (48%) | 36 | (43%) |
| | 118 | | 88 | | 84 | |

Orientation of transposon integrations in
tumors relative to nearest gene

| | | |
|---|---|---|
| In genes | 5′ of gene | 3′ of gene |

| | | | | | | |
|---|---|---|---|---|---|---|
| same | 147 | (62%) | 99 | (65%) | 60 | (51%) |
| inverse | 92 | (38%) | 54 | (35%) | 57 | (49%) |
| | 239 | | 153 | | 117 | |

TABLE 4
Common sites of transposon integration in double transgenic
embryos. Dupuy et al. (manuscript #2005-01-01166C)

| | | | | | | | Number |
|---|---|---|---|---|---|---|---|
| Embryo ID | Gene¹ | Location | Distance | Orientation | Chr | Address | identical² |
| 1 TG6070-SB3C4 | N/D | N/D | N/D | N/D | chr4 | 71257033 | 1 |
| 1 TG6070-SB6B4 | N/D | N/D | N/D | N/D | chr4 | 71280203 | 1 |
| 2 TG6057-SB5B2 | * Stag1 | intron 1 | not disrupt CDS | same | chr9 | 100544128 | 1 |
| 2 TG6113-SB5B6 | * Stag1 | intron 2 | disrupt CDS | inv | chr9 | 100559508 | 1 |
| 3 TG6057-SB5B2 | 5730414C17Rik | 3 prime | 49.257 kb | same | chr13 | 63395267 | 1 |
| 3 TG6057-SB6A2 | 5730414C17Rik | 3 prime | 23.429 kb | same | chr13 | 63421095 | 1 |
| 4 TG6057-SB5A5 | N/D | N/D | N/D | N/D | chr13 | 73334729 | 1 |
| 4 TG6057-SB5B2 | N/D | N/D | N/D | N/D | chr13 | 73344643 | 1 |

¹Symbols: N/D = no gene within 100 kb, * = common retroviral integration site
²Number of identical clones obtained from sequencing 96 independent clones from each sample.
The frequency at which each clone was obtained reflects the percentage of cells that contain the integration.

Double Transgenic Mice are Tumor Prone

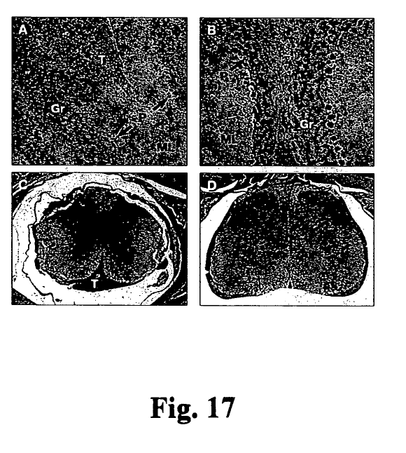

Twenty-four double transgenic mice that survived to weaning were monitored for tumor development. At seven weeks of age the mice began to show signs of illness and by seventeen weeks, all the mice had died from cancer (FIG. 16A). Multiple tumor types were identified with the most common tumor being T-cell lymphoma (FIG. 16B). Tumor cells were frequently found in all tissues of the animal and in some cases a single animal developed two or even three different cancer types (FIG. 16B). Hematopoietic tumors predominated, possibly reflecting the large pool of hematopoietic stem cells present in mice. Medulloblastoma, a solid tumor of the cerebellum, was also observed in two mice while intestinal and pituitary neoplasia was seen in other animals. Thus, unlike retroviral insertional mutagenesis, SB mutagenesis is not limited to the hematopoietic system. Stained sections of the medulloblastoma from animal TG6057-17106 and a corresponding normal cerebellum showed that the normal morphology of the cerebellum was disrupted with tumor cells invading the molecular layer (FIG. 17A). Tumor tissue also extended down the brain stem and could be seen adjacent to the spinal cord (FIG. 17C). This is similar to what is observed in human medulloblastoma.

BamHI-digested tumor DNAs were subsequently analyzed by Southern blotting to determine whether they contained clonal or subclonal SB transpositions. As expected, Southern blotting failed to identify clonal, somatically acquired transposon integrations in tail DNA (FIG. 16C, lane 1) or in DNA from normal brain and kidney (FIG. 13E). In contrast, numerous clonal and subclonal transposon integrations were seen in lymph nodes, spleen and thymus in tumor DNA (FIG. 16C, lane 2-13). The pattern of transposon integrations in different tumor tissues from the same animal was similar but not identical (FIG. 16C, lane 4-6, 12-13), indicating that some transpositions are lost while others are gained during tumor development. These results are consistent with insertional mutagenesis of cancer genes as the disease-inducing mechanism.

Analysis of SB Integration Sites in Tumor DNA

To confirm that these tumors are induced by insertional mutagenesis, 781 SB junctions from 16 tumors were cloned and analyzed. In contrast to the embryo results, multiple genes were identified that were mutated by SB integration in two or more tumors. These results are unlikely to have occurred by chance. Seven of these genes are validated human cancer genes while another seven are mutated by retroviral integration in mouse leukemias (http://rtcgd.ncifcrf.gov). Four genes were identified that were mutated more than once in the same tumor [two Notch1 integrations (TG6057-16315), two Jak1 integrations (TG6070-16887), two Csf3r integrations (TG6070-17306), two Erg integrations (TG6070-17900) and three Erg integrations (TG6070-16881)]. This could reflect tumor microheterogeneity with the different integrations occurring in different subpopulations of tumor cells during tumor progression. Integrations in a number of genes were identified that have not yet been examined for a role in human cancer, but which represent excellent disease gene candidates.

Like the embryo integrations, tumor integrations were widely distributed across the genome with little local hopping. However, integrations in tumor DNA located upstream or within genes showed an orientation bias that was not found in embryo integrations (Table 2). In tumors, 65% of transposons located 5′ of genes are in the same transcriptional orientation as the gene compared to 52% for integrations in embryos (p<0.001). In addition, 62% of transposons located within genes are in the same orientation compared to 54% for integrations in embryos (p<0.001). Unlike retroviruses, which have strong enhancer activity and can activate gene expression over large distances, T2/Onc2 appears to have little enhancer activity. This is supported by the failure to identify common integration sites in which transposons are integrated downstream of the gene and recent data showing that SB transposons that lack viral LTR and corresponding SD sequences fail to significantly increase expression of nearby genes (Yant et al., Mol. Cell Biol., 2005; 25:2085-2094). Consequently, T2/Onc2 primarily activates gene expression by integrating upstream of a gene or in an upstream intron and promoting expression of the gene from the MSCV LTR, or by integrating into the coding region and promoting the expression of a truncated protein or prematurely truncating the transcript. This lack of enhancer activity greatly simplifies the identification of cancer genes mutated by SB.

Activating Notch1 Transpositions

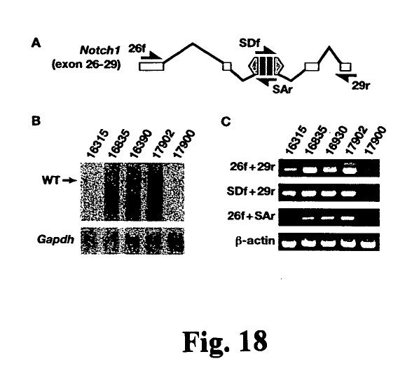

Activating NOTCH1 mutations have been identified in >50% of human T-ALLs (Weng et al., Science, 2003; 306:269-271). Among ten SB-induced T-cell lymphomas analyzed, six contained SB integrations in intron 27 of Notch1 (FIG. 18A). The sequences of the SB-Notch1 splice junction in tumors are provided herein. The splice donor is found in the 17 5′ nucleotides of 5′-CCGCGAGGATCTCTCAGGTGAGCCGGTGGAGCCT-3′ (SEQ ID NO: 92), adjacent to Notch1 exon 28 (SDF+29r), while the splice acceptor is found in the 17 3′ nucleotides of 5′-GATTGAGGCCGTGAAGATTCAGCCGATGATGAAA-3′ (SEQ ID NO: 93), adjacent to Notch1 exon 27 (26f+Sar). These transposon integrations mapped to three different sites in intron 27 indicating that transposition is not totally random or integration at these sites is selected due to their effect on Notch1 expression. All six integrations are oriented in the same transcriptional direction as Notch1 and induce the expression of a Notch1 fusion transcript containing the MSCV promoter and the 3′ end of Notch1 (FIG. 18B,C). This fusion transcript mimics that seen in human T-ALL patients with t(7;9), in which the translocation drives expression of an activated NOTCH1 C-terminal protein fragment (Ellisen et al., Cell, 1991; 66:649-661). Furthermore, transgenic mice overexpressing a similar fragment of Notch1 develop T-cell lymphoma (Beverly et al., Cancer Cell, 2003; 3:551-564). These results confirm that SB-induced tumors are induced by insertional mutagenesis.

Cooperating Cancer Genes and Pathways

Among the six tumors with activating integrations at Notch1, three also had activating integrations upstream of Rasgrp1 (FIG. 19, a gene that positively regulates Ras signaling. The probability of finding two tumors with integrations in the same two pair of genes by chance is low (p=9.2×10⁻⁵, see Experimental Methods). Integrations in Rasgrp1 were only seen in tumors with Notch1 integrations, suggesting that Ras signaling cooperates with Notch1 in tumor induction. Two tumors with Notch1 and Rasgrp1 integrations also had activating integrations upstream of Sox8, an uncharacterized member of the Sox family of SRY-related HMG-box DNA-binding proteins (FIG. 19). The probability of finding two tumors with integrations in Notch1, Rasgrp1 and Sox8 by chance is exceedingly low (p=2.2×10⁻⁷) and suggests that Sox8 could represent another signaling pathway that cooperates with Notch1 in tumor induction. Finally, two Notch1 tumors also have activating integrations upstream of Runx2 (FIG. 19), suggesting that Runx2 might represent yet another Notch1-cooperating gene.

Although the majority of genes mutated by SB transposition in tumors were identified in only one tumor, several were identified that belong to related signaling pathways. Careful annotation identified seven pathways that were commonly disrupted in SB-induced tumors (Table 4). Similar analysis of the integration sites cloned from embryos did not reveal any similar trends. Integrations in most cases are predicted to affect a given pathway in a similar manner but accomplish this through the disruption of different genes. For example, six tumors have transposon-induced mutations that are predicted to result in decreased TNF signaling. Similarly, decreased rates of receptor recycling, increased signaling through the Ras superfamily, increased Jak/Stat signaling and increased Wnt signaling are all common pathways affected by SB transposition in tumors (Table 5). Identification of genes and signaling pathways that cooperate to induce cancer will make it possible to develop better combinatorial therapies for treating human cancer.

TABLE 5
Pathways commonly affected by transposon integration.

| | | Predicted |
|---|---|---|
| | | effect on |
| Pathway | Genes | pathway |
| Tumor necrosis | Tank, Mtx2, Tnfrsf26, Tnfrsf11a, | decrease |
| factor | Tnfrsf1b, Edar, Pde4b | |
| Receptor recycling | Eps15, Rab11a, Rabgap1l, Vps26, | decrease |
| | Sept2 | |
| Cellular transport | Kif16b, Ank3, Kifap3, Sec8l1, | unknown |
| | Ralbp1 | |
| Ras superfamily | Rapgef2, Rasgrp1, Ralgps1, Rap1b, | increase |
| | Sos1, Ralbp1, Sipa1l1, Eras, Kras2 | |
| Jak/Stat | Jak1, Stat5b | increase |
| Ets transcription | Ets1, Erg, Fli1 | increase |
| factors | | |
| Wnt signaling | Fzd7, Wnt7b, En1, Musk, Catnbip1 | increase |

Experimental Methods
Generation of the RosaSB Allele

An expression cassette consisting of an En2 splice acceptor, SB11 cDNA and SV40 polyA was cloned upstream of a floxed PGKneo cassette. The cassette was then recombined into a plasmid containing a TK selection cassette as well as the promoter region, exon 1 and a portion of the single intron of the Rosa26 locus using the “recombineering” strategy previously described (Liu et al., Genome Res., 2003; 13:476-84). This recombination introduced the knock-in cassette into the XbaI site of the Rosa26 intron that has been used in previous Rosa26 knock-in alleles (Soriano et al., Nat. Genet., 1999; 21:70-71). The targeting plasmid was then linearized and introduced into embryonic stem (ES) cells. Following selection, ES cell colonies were picked, DNA extracted and digested with SpeI to screen the 5′ region of Rosa26 and BglI for the 3′ region. Southern blotting was performed on the 5′ region using a 908 bp SacI fragment of the Rosa26 promoter region. A 667 bp SspI fragment derived from the intron of Rosa26 was used to confirm the 3′ recombination site by Southern analysis. Three independent clones were injected into blastocysts to derive three RosaSB knock-in lines. Mice were genotyped by PCR using primers specific to the SB11 cDNA: 5′-ATGGGAAAATCAAAAGAAATCAGCCAAG-3′ (SEQ ID NO:64) and 5′-GCCAAACAGTTCTATTTTTGTTTCATCAGACCA-3′ (SEQ ID NO:65). One line was subsequently maintained by backcrossing to C57BL/6 mice.

Generation of T2/Onc2 Transgenic Mice

A plasmid containing the T2/Onc transposon was obtained from David Largaespada (University of Minnesota). The T2/Onc2 transposon was made by replacing the HpaI/BglII fragment containing the En2 splice acceptor from pT2/Onc with a fragment containing a larger portion of the En2 exon. In addition to this change, the overall size of T2/Onc2 was reduced (2050 bp compared to 2163 bp for T2/Onc) but was otherwise identical to T2/Onc. The pT2/Onc2 plasmid was linearized using ScaI and prepared for microinjection into (B6C3)F2 hybrid embryos using standard techniques. Tail biopsy DNA from founder animals was screened by Southern blotting using an En2 splice acceptor probe. Transgenic lines were established by crossing to C57BL/6. Offspring were genotyped by PCR using primers 5′-CAGTTG AAGTCGGAAGTTTA-3′ (SEQ ID NO:66) and 5′-GGAATTGTGATACAGTGAAT-3′ (SEQ ID NO:67).

Calculation of Expected Number of Common Oil Sites

A JAVA program was created to simulate the random SB transposon insertions in the mouse genome. The program randomly selected 491 (number of sites cloned from embryos) or 782 (number of sites cloned from tumors) TA motifs from whole mouse genome by using random number generator. The program then counted the number of common integration sites by calculating distances between the integration sites. After repeating this procedure 10,000 times, the average expected number of common integration sites was determined.

Southern Blotting on Embryo and Tumor DNA

Genomic DNA was digested with BamHI and blotted using standard techniques. The portion of the blot below 600 bp was trimmed away and analyzed separately as it contained the signal from transposons remaining within the transgene concatamer. PCR was performed to generate a 278 bp probe from the region of pT2/Onc2 between the IRDRL and the MSCV promoter (5′-GGATCCACTAAATTCC-3′ (SEQ ID NO:68)) and 5′-GTTGACTGTGCCTTTA-3′ (SEQ ID NO:69)). This region is unique sequence not found in the mouse genome. Southern blotting was performed using standard techniques.

LM-PCR Cloning

Approximately 1 μg of genomic DNA was digested with NlaIII (IRDRR) or BfaI (IRDRL). Digested DNA was then purified using a Qiagen column (QIAquick PCR purification) and digested with XhoI (IRDRR) or BamHI (IRDRL). This was done to eliminate amplification of transposon junctions within the transgene concatamer. Digested DNA was again purified using a Qiagen column (QIAquick PCR purification), and a 5 μl aliquot was added to a ligation reaction containing 150 μmoles of a doublestranded linker. Linkers were generated by annealing equimolar amounts of NlaIII linker+(5′-GTAATACGACTCACTATAGGGCTCCGCTTAAGGGACCATG-3′ (SEQ ID NO:70)) and NlaIII linker-(5′-Phos-GTCCCTTAAGCGGAG-C3spacer-3′ (SEQ ID NO:71)) for the IRDRR. The 5′ phosphate modification of the linker-oligo aids ligation of the linker, and the C3 spacer modification at the 3′end of the linker-oligo prevents priming of Taq polymerase. The linker for the IRDRL was generated using equimolar amounts of BfaI linker+(5′-GTAATACGAC TCACTATAGGGCTCCGCTTAAGGGAC-3′ (SEQ ID NO:72)) and BfaI linker-(5′-Phos-TAGTCCCTTAAGCG GAG-C3spacer-3′ (SEQ ID NO:73)). Ligations were performed using high concentration T4 ligase (NEB) at room temperature for 2-3 hours. Primary PCR was performed using high fidelity Platinum Taq (Invitrogen) and the linker primer (5′-GTAATACGACTCACTATAGGGC-3′ (SEQ ID NO:74)) and IRDRR1 (5′-GCTTGTGGAAGGCTACTCGAAATGTTTGACCC-3′ (SEQ ID NO:75)) or IRDRL1 (5′-CTGGAATTTTCCAAGCTGTTTAAAGGCACAGTCAAC-3′ (SEQ ID NO:76)). Cycle conditions were as follows: 94° C. for 2 minutes, 94° C. for 15 seconds, 60° C. for 30 seconds and 72° C. for 1 minute for 25 cycles followed by a final extension at 72° C. for 5 minutes. Primary PCR products were then diluted 1:50 in H₂O, and a 2 μl aliquot of the dilution was used for secondary PCR. Secondary PCR was performed using the linker nested primer (5′-AGGGCTCC GCTTAAGGGAC-3′ (SEQ ID NO:77)) and IRDRR2 (5′-CCACTGGGAATGTGATGAAAGAAATAAAAGC-3′ (SEQ ID NO:78)) or IRDRL2 (5′-GACTTGTGTCATGCACAAAGTAGATGTCC-3′ (SEQ ID NO:79)). Cycle conditions for secondary PCR were identical to the primary PCR. Secondary PCR products were then cleaned up using a Qiagen column (QIAquick PCR purification). A 3 μl aliquot of each sample was then ligated into the pGEM-T Easy vector (Promega) using high concentration T4 ligase (NEB) and transformed into Electromax DH10B cells (Invitrogen). Colonies were selected on ampicillin plates containing x-gal for blue-white screening. Ninety-six white colonies were picked from each sample and prepared for sequencing using the Qiagen DirectPrep 96 kit. Each clone was then sequenced using the SP6 sequencing primers.

Northern and R7-PCR Analysis

Total RNA was extracted from tumor samples using the RNA STAT-60 reagent (Tel-Test, Inc). The total RNA was then polyA selected using the MicroPoly(A)Purist Kit (Ambion, Inc.). Northern blotting was performed using polyA+RNA and subsequently probed using an 882 bp NotI fragment of the Notch1 cDNA. The blot was then allowed to decay and then hybridized with a Gapdh probe. RTPCR was performed using the One-Step RT-PCR kit (Qiagen, Inc.) with these primers: SDf (5′-CTACTAGCACCAGAACGCCC-3′ (SEQ ID NO:80)), 26f (5′-TGGACCCCATGGACA T-3′ (SEQ ID NO:81)), 29r (5′-TGCAGTCAGCATCCACCTCC-3′ (SEQ ID NO:82)), SAr (5′-CATCTTTCACATACCGGCTA-3′ (SEQ ID NO:83)), β-actin forward (5′-GTGGGCCGCCCTAGGCACCA-3′ (SEQ ID NO:84)) and β-actin rev (5′-CTCTTTGATGTCACGCACGA-3′ (SEQ ID NO:85)) as described.

Quantitative PCR and RT-PCR

The polyA+ RNA was used as template in a cDNA synthesis reaction using the Superscript™ First-Strand cDNA Synthesis System (Invitrogen, Inc.). Quantitative PCR was performed on Rasgrp1 (5′-GCTGATATTTTCACTGGGGA-3′ (SEQ ID NO:86) and 5′-CCTGCGTGAATAGACCCTGA-3′ (SEQ ID NO:87)) and Runx2 (5′-AACTGCCTGGGGTCTGAAAA-3′ (SEQ ID NO:88) and 5′-CCTCAGTGATTTAGGGCGCA-3′ (SEQ ID NO:89)) using SYBR green technology. RTPCR was performed on Sox8 (5′-GCTCCGTCTTGATCTGTGGC-3′ (SEQ ID NO:90) and 5′-GACCACCACACAGGCCAGAC-3′ (SEQ ID NO:91)) using the One-Step RTPCR kit (Qiagen, Inc.)

Calculations Used to Determine the Expected Frequencies of Common Integration Site Pairs in Independent Tumors.

This calculation focuses on distinct doubles (AB, BC, CD, etc.) and distinct triples (ABC, etc.). The calculations were carried out to determine the probability that any distinct double or triple would be repeated over 15 additional tumors. To achieve this, it was found that the probability that any distinct double will be observed x times over 15 tumors containing 50 random integrations, where X˜BIN (15, p) where p=6.125*10⁻⁶. Fifty random integrations were chosen, since this is the average number of transposon integrations in each tumor. The probability p is based on the fact that a distinct double, arbitrary and predetermined by occurrence on one tumor can occur 50*49*(20,000⁴⁸) ways on any other tumor. However there are (20,000⁵⁰) possible patterns of mutations that can occur on a single tumor, thus p=(50*49)/400,000,000=6.125*10⁻⁶. Likewise, the probability that a distinct triple, arbitrary and predetermined by occurrence on one tumor can occur 50*49*48*(20,000⁴⁷) ways on any other tumor and thus since there are (20,000⁵⁰) possible patterns of mutations that can occur on a single tumor, for a triple, p=50*49*48/(8,000,000,000,000)=1.47*10⁻⁸. Employing the binomial distribution and SPSS the results for x ranging from 0 to 15 are given in the Table 6 below:

TABLE 6

| # replications | Pr | # replications | Pr |
|---|---|---|---|
| distinct pair | (# replications) | distinct triple | (# replications) |
| 0 | 0.999908129 | 0 | 0.99999978 |
| 1 | 0.000091867 | 1 | 0.00000022 |
| 2 | 0.000000004 | 2 | 0.00000000 |
| 3 | 0.000000000 | 3 | 0.00000000 |
| 4 | 0.000000000 | 4 | 0.00000000 |
| 5 | 0.000000000 | 5 | 0.00000000 |
| 6 | 0.000000000 | 6 | 0.00000000 |
| 7 | 0.000000000 | 7 | 0.00000000 |
| 8 | 0.000000000 | 8 | 0.00000000 |
| 9 | 0.000000000 | 9 | 0.00000000 |
| 10 | 0.000000000 | 10 | 0.00000000 |
| 11 | 0.000000000 | 11 | 0.00000000 |
| 12 | 0.000000000 | 12 | 0.00000000 |
| 13 | 0.000000000 | 13 | 0.00000000 |
| 14 | 0.000000000 | 14 | 0.00000000 |
| 15 | 0.000000000 | 15 | 0.00000000 |

Thus, in the case of either a distinct double (AB, etc.) or a distinct triple (ABC, etc.), provided it has occurred on one tumor, the probability is overwhelmingly high that it shall not reoccur (well over 99%). The chances a distinct double will reoccur once are only about 9 in 100,000 and that a distinct triple will reoccur is even less (about 2 in 10,000,000).

### Example 3

### Transposition Assay

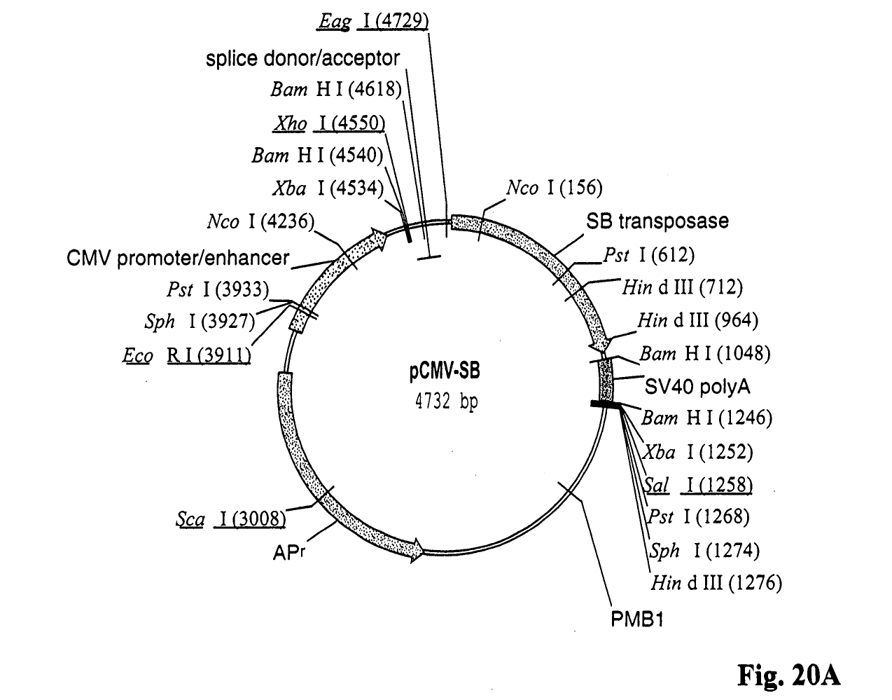

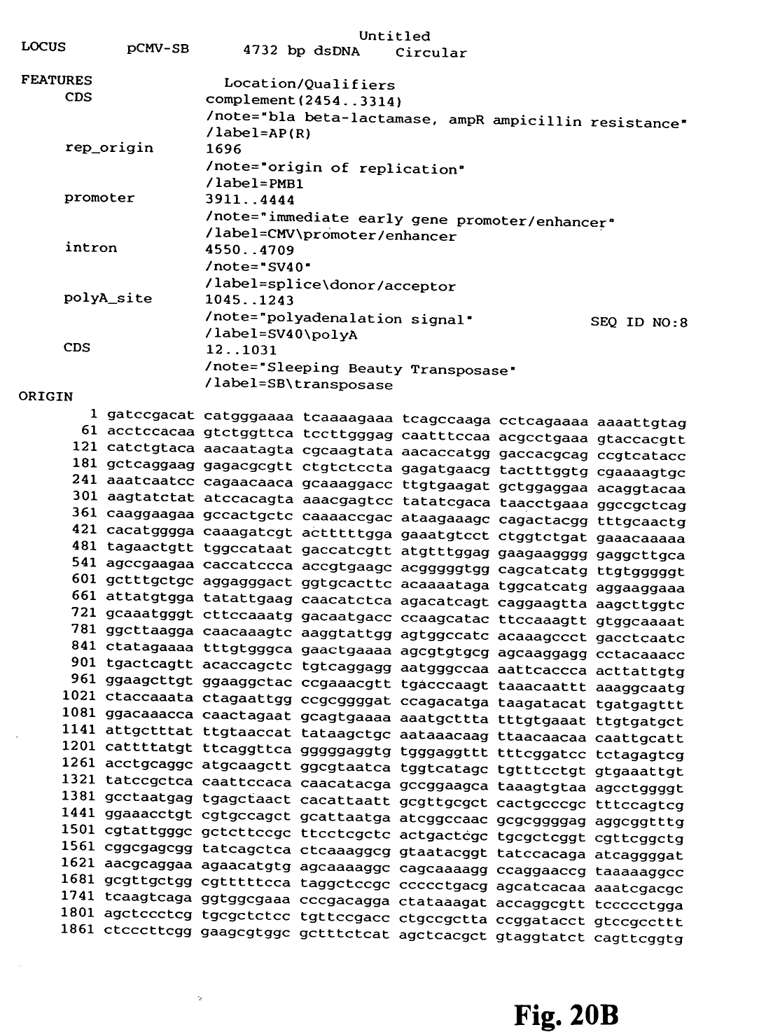

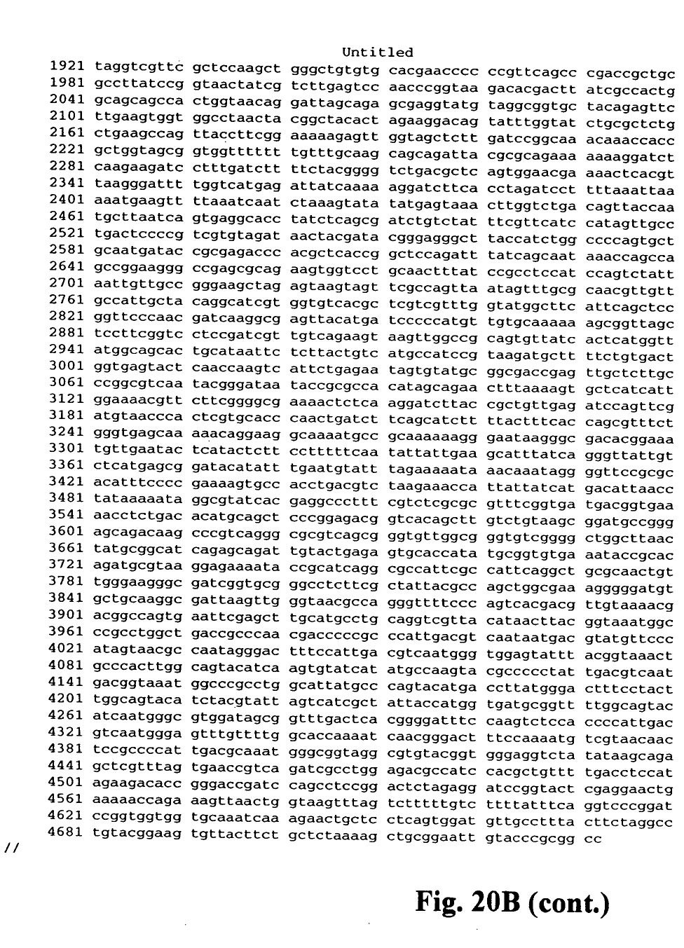

An assay may be used to measure the level of excision and reintegration (transposition) provided by a transposition system. Preferably, the assay for measuring transposition uses a mammalian cell line, preferably HeLa cells. The cells can be cultured using routine methods, preferably by culturing in DMEM supplemented with about 10% fetal bovine serum (for instance, characterized fetal bovine serum, available from Hyclone, Logan, Utah), about 2 mM L-glutamine, and antibiotics (for instance, antimycotic, available from Gibco-BRL, Carlsbad, Calif.). Typically, the cells are seeded at a density of about 3×10⁵ cells per 6-cm plate one day prior to transfection. The cells are transfected with from about 450 ng to about 550 ng, preferably about 500 ng vector containing the transposon, and from about 450 ng to about 550 ng, preferably 500 ng of vector encoding the SB transposase. Preferably, the vector pCMV-SB (SEQ ID NO:8) is used as the source of SB transposase (FIG. 20 A,B) Methods for transfecting mammalian cells with DNA are routine. Preferably, the transfection reagent TransIT-LTI (available from Mirus, Madison, Wis.) is used. At about 24 hours post transfection, cells are typically washed with 1×PBS and fresh medium added. At about 2 days post-transfection, the transfected cells are typically trypsinized, resuspended in serum-containing DMEM, and about 3×10⁴ cells may be seeded onto several 10 cm plates in medium, supplemented with the appropriate selective agent if necessary. After about two to about three weeks of growth, the number of colonies expressing the marker are counted. For instance, when the transposon encodes resistance to the neomycin analog G418, the cells can be fixed with about 10% formaldehyde in PBS for about 15 minutes, stained with methylene blue in PBS for bout 30 minutes, washed extensively with deionized water, air dried and counted.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

All headings are for the convenience of the reader and should not be used to limit the meaning of the text that follows the heading, unless so specified.

What is claimed is:

1. A method for characterizing an insertional mutation in a tumor-bearing mammal, comprising:
providing a transgenic mammal, wherein a cell of the transgenic mammal comprises:
a polynucleotide comprising a coding region encoding a transposase, and
a transposon comprising a polynucleotide, or complement thereof, comprising an insertional mutagen flanked by first and second inverted repeats, wherein the inverted repeats can bind to a transposase and the transposon is capable of integrating into genomic DNA in a cell;
obtaining a tumor cell from a tumor on the transgenic mammal; and
identifying the location of a mobilized transposon in the genomic DNA of the tumor cell.

2. The method of claim 1, wherein the first inverted repeat comprises a first outer direct repeat and a first inner direct repeat, the first outer direct repeat comprising a nucleotide sequence having at least about 80% identity to SEQ ID NO:3, and the first inner direct repeat comprising a nucleotide sequence having at least about 80% identity to SEQ ID NO:4, and each direct repeat binds an SB polypeptide, and
wherein the second inverted repeat comprises a second inner direct repeat and a second outer direct repeat, the second inner direct repeat comprising a complement of a nucleotide sequence having at least about 80% identity to SEQ ID NO:4, and the second outer direct repeat comprising a complement of a nucleotide sequence having at least about 80% identity to SEQ ID NO:3, and each direct repeat binds an SB polypeptide; and
wherein the transposase is an SB transposase.

3. The method of claim 1, wherein the transposase comprises an amino acid sequence having at least about 80% identity with SEQ ID NO:21.

4. The method of claim 1, wherein identifying the location of a mobilized transposon comprises determining the nucleotide sequences adjacent to the mobilized transposon.

5. The method of claim 1, wherein the locations of a plurality of mobilized transposon are identified in the genomic DNA of the tumor cell.

6. The method of claim 1, wherein tumor cells are obtained from a plurality of transgenic mammals.

7. The method of claim 6, further comprising comparing the locations of mobilized transposon from tumors obtained from different transgenic mammals to identify the location of a common insertion site.

8. The method of claim 1, wherein the transgenic mammal is genetically predisposed to develop cancer.

9. The method of claim 1, wherein the insertional mutagen comprises an affective sequence, a disruptive sequence, or a combination thereof.

10. The method of claim 1, wherein the insertional mutagen comprises a splice acceptor site, a promoter, a splice donor site, a transcription terminator, or a combination thereof.

11. The method of claim 1, wherein the tumor is a solid tumor.

12. A method for identifying a common insertion site, comprising:
identifying the location of a mobilized transposon in the genomic DNA of a tumor cell from a first transgenic mammal and a second transgenic mammal, comprising:
providing a first and second transgenic mammal, wherein a cell of the transgenic mammal comprises:
a polynucleotide comprising a coding region encoding a transposase, and
a transposon comprising a polynucleotide, or complement thereof, comprising an insertional mutagen flanked by first and second inverted repeats, wherein the inverted repeats can bind to the transposase and wherein the transposon is capable of integrating into genomic DNA in a cell;
obtaining genomic DNA from a tumor cell from the first and second transgenic mammal;
determining the nucleotide sequences adjacent to the mobilized transposon to identify the location of the mobilized transposon in the genomic DNA of the tumor cell from the first and second transgenic mammals;
comparing the location of the mobilized transposon obtained from the genomic DNA of the first and second transgenic mammals, wherein the presence of the mobilized transposon in the same genomic region in both transgenic mammals indicates the genomic region is a common insertion site.

13. The method of claim 12, wherein
wherein the first inverted repeat comprises a first outer direct repeat and a first inner direct repeat, the first outer direct repeat comprising a nucleotide sequence having at least about 80% identity to SEQ ID NO:3, and the first inner direct repeat comprising a nucleotide sequence having at least about 80% identity to SEQ ID NO:4, and each direct repeat binds an SB polypeptide, and
wherein the second inverted repeat comprises a second inner direct repeat and a second outer direct repeat, the second inner direct repeat comprising a complement of a nucleotide sequence having at least about 80% identity to SEQ ID NO:4, and the second outer direct repeat comprising a complement of a nucleotide sequence having at least about 80% identity to SEQ ID NO:3, and each direct repeat binds an SB polypeptide; and
wherein the transposase is an SB transposase.

14. The method of claim 12, wherein the transposase comprises an amino acid sequence having at least about 80% identity with SEQ ID NO:21.

15. The method of claim 12, wherein the insertional mutagen comprises an affective sequence and a disruptive sequence.

16. The method of claim 15, wherein the affective sequence comprises a splice donor and a promoter, and the disruptive sequence comprise a splice acceptor operably linked to a transcription termination signal site.

17. The method of claim 13, wherein the insertional mutagen comprises nucleotides 533 to 630, 807 to 1207, 1217 to 1394, 1444-1525, and 1686 to 1959 of SEQ ID NO:19.

18. The method of claim 12, wherein the common insertion site comprises the integration of two mobilized transposons identified from tumor cells obtained from two transgenic mammals within about 13 kilobases of each other.

19. The method of claim 12, further comprising a third transgenic mammal, wherein the common insertion site comprises the integration of three mobilized transposons identified from tumor cells obtained from three transgenic mammals within about 269 kilobases of each other.

20. The method of claim 12, wherein the common insertion site has a high probability of being a nucleotide sequence within a tumor-associated gene.
